(19) United States
(12) Patent Application Publication
(10) Pub. No.: US 2020/0038414 A1
(43) Pub. Date: Feb. 6, 2020

(54) METHODS OF TREATING CONSTIPATION USING AMINOSTEROL COMPOSITIONS

(71) Applicant: Enterin, Inc., Philadelphia, PA (US)

(72) Inventors: Denise Barbut, Philadelphia, PA (US); Michael Zasloff, Philadelphia, PA (US)

(73) Assignee: Enterin, Inc., Philadelphia, PA (US)

(21) Appl. No.: 16/530,127

(22) Filed: Aug. 2, 2019

Related U.S. Application Data
(60) Provisional application No. 62/714,470, filed on Aug. 3, 2018.
(60) Provisional application No. 62/714,468, filed on Aug. 3, 2018.
(60) Provisional application No. 62/720,453, filed on Aug. 21, 2018.

(51) Int. Cl. A61K 31/575; A61K 9/00; A61P 1/10

(52) U.S. Cl. CPC A61K 31/575; A61P 1/10; A61K 9/0053; A61K 9/0043

(57) ABSTRACT

This invention relates to methods of treating constipation and constipation-related symptoms in a subject in need. The methods, which utilize a patient-specific “fixed dose” of an aminosterol, comprise administering a therapeutically effective dose of an aminosterol to a subject in need.

### CROSS-REFERENCE TO RELATED APPLICATIONS

This application claims the priority benefits under 35 USC § 119 to U.S. provisional Application No. 62/714,470, filed Aug. 3, 2018; U.S. provisional Application No. 62/714,468, filed Aug. 3, 2018; and U.S. provisional Application 62/720,453, filed Aug. 21, 2018, the entire contents of which are incorporated herein by reference in their entirety.

### FIELD OF THE INVENTION

This invention relates to methods of treating constipation, which can be associated with various diseases or conditions.

### BACKGROUND

Amino sterols are amino derivatives of a sterol. Examples of aminosterols include squalamine and Aminosterol 1436 (also known as trodusquemine and MSI-1436).

Squalamine is a unique compound with a structure of a bile acid coupled to a polyamine (spermidine):

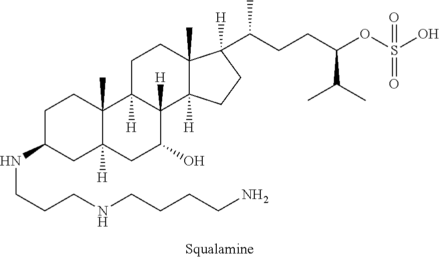

The discovery of squalamine was reported by Michael Zasloff in 1993 (U.S. Pat. No. 5,192,756). Squalamine was discovered in various tissues of the dogfish shark (Squalus acanthias) in a search for antibacterial agents. The most abundant source of squalamine is in the livers of Squalus acanthias, although it is found in other sources, such as lampreys (Yun et al. 2007).

Several clinical trials have been conducted relating to the use of squalamine, including the following:

(1) ClinicalTrials.gov Identifier NCT01769183 for “Squalamine for the Treatment in Proliferative Diabetic Retinopathy,” by Elman Retina Group (6 participants; study completed August 2014);

(2) ClinicalTrials.gov Identifier NCT02727881 for “Efficacy and Safety Study of Squalamine Ophthalmic Solution in Subjects With Neovascular AMD (MAKO),” by Ohr Pharmaceutical Inc. (230 participants; study completed December 2017);

(3) ClinicalTrials.gov Identifier NCT02614937 for “Study of Squalamine Lactate for the Treatment of Macular Edema Related to Retinal Vein Occlusion,” by Ohr Pharmaceutical Inc. (20 participants; study completed December 2014);

(4) ClinicalTrials.gov Identifier NCT01678963 for “Efficacy and Safety of Squalamine Lactate Eye Drops in Subjects With Neovascular (Wet) Age-related Macular Degeneration (AMD),” by Ohr Pharmaceutical Inc. (142 participants; study completed March 2015);

(5) ClinicalTrials.gov Identifier NCT00333476 for “A Study of MSI-1256F (Squalamine Lactate) To Treat “Wet” Age-Related Macular Degeneration,” by Genaera Corporation (140 participants; study terminated);

(6) ClinicalTrials.gov Identifier NCT00094120 for “MSI-1256F (Squalamine Lactate) in Combination With Verteporfin in Patients With ‘Wet’ Age-Related Macular Degeneration (AMD),” by Genaera Corporation (60 participants; study completed February 2007);

(7) ClinicalTrials.gov Identifier NCT00089830 for “A Safety and Efficacy Study of MSI-1256F (Squalamine Lactate) To Treat ‘Wet’ Age-Related Macular Degeneration,” by Genaera Corporation (120 participants; study completed May 2007); and

(8) ClinicalTrials.gov Identifier NCT03047629 for Evaluation of Safety and Tolerability of ENT-01 for the Treatment of Parkinson's Disease Related Constipation (RASMET) (50 participants; study completed Jun. 14, 2018).

Aminosterol 1436 is an aminosterol isolated from the dogfish shark, which is structurally related to squalamine (U.S. Pat. No. 5,840,936; Rao, Shinnar et al. 2000). Amino sterol 1436 has an additional pharmacological property, not shared with squalamine, namely potent appetite suppression and promotion of dose-dependent weight loss (U.S. Pat. No. 6,143,738; Ahima et al. 2002).

Several clinical trials have been conducted relating to the use of Aminosterol 1436:

(1) ClinicalTrials.gov Identifier NCT00509132 for “A Phase I, Double-Blind, Randomized, Placebo-Controlled Ascending IV Single-Dose Tolerance and Pharmacokinetic Study of Trodusquemine in Healthy Volunteers,” by Genaera Corp.;

(2) ClinicalTrials.gov Identifier NCT00606112 for “A Single Dose, Tolerance and Pharmacokinetic Study in Obese or Overweight Type 2 Diabetic Volunteer,” by Genaera Corp.;

(3) ClinicalTrials.gov Identifier NCT00806338 for “An Ascending Multi-Dose, Tolerance and Pharmacokinetic Study in Obese or Overweight Type 2 Diabetic Volunteers,” by Genaera Corp.; and

(4) ClinicalTrials.gov Identifier: NCT02524951 for “Safety and Tolerability of MSI-1436C in Metastatic Breast Cancer,” by DepyMed Inc.

Even in view of these trials, the full potential of aminosterols for use in treatment has yet to be determined.

### SUMMARY OF THE INVENTION

The present invention is directed to methods of treating constipation, constipation-related symptoms, and constipation-related conditions and diseases.

In one aspect, described is a method of treating, preventing, and/or slowing the onset or progression of constipation and/or a constipation-related symptom in a subject in need comprising administering to the subject a therapeutically effective amount of at least one aminosterol, or a salt or derivative thereof, provided that the administering does not comprise oral administration. For example, the method can comprise administration selected from nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof. In one aspect, administering comprises nasal administration.

The therapeutically effective amount of the at least one aminosterol or a salt or derivative thereof: (a) comprises about 0.1 to about 20 mg/kg body weight of the subject; and/or (b) comprises about 0.1 to about 15 mg/kg body weight of the subject; and/or (c) comprises about 0.1 to about 10 mg/kg body weight of the subject; and/or (d) comprises about 0.1 to about 5 mg/kg body weight of the subject; and/or (e) comprises about 0.1 to about 2.5 mg/kg body weight of the subject; and/or (f) comprises about 0.001 to about 500 mg/day; and/or (g) comprises about 0.001 to about 250 mg/day; and/or (h) comprises about 0.001 to about 125 mg/day; and/or (i) comprises about 0.001 to about 50 mg/day; and/or (j) comprises about 0.001 to about 25 mg/day; and/or (k) comprises about 0.001 to about 10 mg/day; and/or (l) comprises nasal administration and wherein the therapeutically effective amount of the at least one aminosterol, or a salt or derivative thereof comprises about 0.001 to about 6 mg/day; and/or (m) comprises nasal administration and wherein the therapeutically effective amount of the at least one aminosterol, or a salt or derivative thereof comprises about 0.001 to about 4 mg/day; and/or (n) comprises nasal administration and wherein the therapeutically effective amount of the at least one aminosterol, or a salt or derivative thereof comprises about 0.001 to about 2 mg/day; and/or (o) comprises nasal administration and wherein the therapeutically effective amount of the at least one aminosterol, or a salt or derivative thereof comprises about 0.001 to about 1 mg/day.

In one embodiment of the disclosure, the aminosterol or a salt or derivative thereof is taken on an empty stomach, optionally within two hours of the subject waking; and/or no food is taken or consumed after about 60 to about 90 minutes of taking the aminosterol or a salt or derivative thereof.

In another aspect, the aminosterol or a salt or derivative thereof is a pharmaceutically acceptable grade of at least one aminosterol or a pharmaceutically acceptable salt or derivative thereof. In yet another aspect, the subject is human. In addition, the subject can be a member of a patient population or an individual at risk for developing constipation

The aminosterol can be comprised in a composition further comprising one or more of the following: an aqueous carrier; a buffer; a sugar; and/or a polyol compound.

In one embodiment, the present invention is directed to methods of treating constipation and/or a constipation-related symptom in a subject in need, comprising (a) determining a dose of an aminosterol or a salt or derivative thereof for the subject, wherein the aminosterol dose is determined based on the effectiveness of the aminosterol dose in improving or resolving constipation and/or the constipation-related symptom in the subject; (b) followed by administering the dose of the aminosterol or a salt or derivative thereof to the subject for a period of time. The method of determining the aminosterol dose comprises (i) identifying a constipation-related symptom to be evaluated; (ii) identifying a starting aminosterol dose for the subject; and (iii) administering an escalating dose of the aminosterol to the subject over a defined period of time until an effective aminosterol dose is identified, wherein the effective aminosterol dose is the dose where improvement or resolution of the constipation-related symptom is observed, and fixing the aminosterol dose at that level in that particular subject. Optionally, each defined period of time is independently selected from the group consisting of about 1 day to about 10 days, about 10 days to about 30 days, about 30 days to about 3 months, about 3 months to about 6 months, about 6 months to about 12 months, and about greater than 12 months.

In an exemplary embodiment, the aminosterol or a salt or derivative thereof is a pharmaceutically acceptable grade of the aminosterol or a salt or derivative thereof.

The constipation-related symptom can be any known symptom of constipation. For example, the symptom can be selected from the group consisting of frequency of constipation, duration of constipation symptoms, frequency of bowel movements, fecal incontinence/encopresis, abdominal pain, abdominal distension or bloating, abdominal discomfort, stomach cramps, stool consistency, painful defecation/rectal pain with bowel movement, rectal burning during or after bowel movement, rectal bleeding or tearing during or after a bowel movement, ease of defecation/passing stool, straining during defecation and/or straining or squeezing to try to pass bowel movements, incomplete evacuation or bowel movement, unsuccessful attempts at evacuation, sensation of incomplete bowel evacuation, sensation of anorectal obstruction/blockage, bowel movements that were too hard, bowel movements that were too small, change in amount of gas passed rectally, less frequent bowel movements, oozing liquid stool, rectal fullness or pressure, small stool size, urge but inability to pass stool, or personal judgement of constipation.

In one aspect, where the constipation symptom to be evaluated is frequency of bowel movements, the fixed escalated aminosterol dose causes the subject to have a bowel movement; and/or the method results in an increase in the frequency of bowel movement in the subject. In another aspect, the method results in an increase in the frequency of bowel movement in the subject and the increase in the frequency of bowel movement is defined as: (i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or.

In another aspect, as a result of the method the subject has the frequency of bowel movement recommended by a medical authority for the age group of the subject.

In one embodiment, the starting aminosterol dose is determined by the severity of the constipation, wherein: (i) if the average complete spontaneous bowel movement (CSBM) or spontaneous bowel movement (SBM) is one or less per week, then the starting aminosterol dose is at least about 150 mg; and (ii) if the average CSBM or SBM is greater than one per week, then the starting aminosterol dose is about 75 mg or less.

In another embodiment, the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%. The improvement can be measured, for example, using a clinically recognized scale or tool.

In one embodiment, the constipation-related symptom is frequency of bowel movements, and the improvement or resolution comprises a desired rate of complete spontaneous bowel movement (CSBM) or spontaneous bowel movement (SBM). In another embodiment, the constipation-related symptom is frequency of bowel movements, and the improvement or resolution comprises a rate of CSBM or SBM in the subject of one or more CSBM or SBM per week, 2 or more CSBM or SMB per week, or 3 or more CSBM or SBM per week. In yet a further embodiment, the improvement or resolution comprises an increase in bowel activity, an induction of nausea, an induction of secretory diarrhea, or any combination thereof.

In the methods of the invention, the aminosterol or a salt or derivative thereof can be administered via any pharmaceutically acceptable means, such as orally, intranasally, by injection, or any combination thereof.

In one embodiment, the starting dose of an aminosterol or a salt or derivative thereof is based on a baseline rate of complete spontaneous bowel movement (CSBM) or spontaneous bowel movement (SBM) in the subject. In another embodiment, the starting dose of the aminosterol or a salt or derivative thereof is higher if the constipation is severe, where “severe” is defined as less than one CSBM or SBM per week.

In an exemplary embodiment, the aminosterol or a salt or derivative thereof is administered orally and the starting aminosterol dose ranges from about 1 mg up to about 175 mg. For example, the starting oral aminosterol dose can be about 25 mg/day.

In another embodiment, the aminosterol or a salt or derivative thereof is administered orally and the dose of the aminosterol or a salt or derivative thereof for the subject following escalation is fixed at a range of from about 1 mg up to about 500 mg. For example, the aminosterol or a salt or derivative thereof can be administered orally and the dose of the aminosterol or a salt or derivative thereof for the subject following dose escalation can be fixed at a dose of about 1, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, or about 500 mg/day.

In another embodiment, a subject experiencing moderate constipation or a related symptom, which is defined as a baseline rate of CSBM or SBM in the subject of one or more CSBM or SBM per week, is administered a starting oral dose of aminosterol or a salt or derivative thereof of from about 10 to about 75 mg/day. For example, in this embodiment the starting oral aminosterol dose can be about 10, about 15, about 20, about 25, about 30, about 35, about 40, about 45, about 60, about 65, about 70, or about 75 mg/day.

In yet another embodiment, a subject experiencing severe constipation or a related symptom, which is defined as a baseline rate of CSBM or SBM in the subject of less than one CSBM or SBM per week, is administered a starting oral aminosterol dose of at least about 75 mg/day. For example, in this embodiment the starting oral aminosterol dose may be from about 75 to about 175 mg/day, or higher. For example, the starting oral aminosterol dose may be about 75, about 80, about 85, about 90, about 95, about 100, about 105, about 110, about 115, about 120, about 125, about 130, about 135, about 140, about 145, about 150 about 155, about 160, about 165, about 170, or about 175 mg/day. In one aspect of this embodiment, the starting oral aminosterol dose is at least about 175 mg/day.

In the methods of the invention, where the aminosterol or a salt or derivative thereof is administered orally, the dose of the aminosterol or a salt or derivative thereof can be escalated in increments of, for example, about 5, about 10, about 15, about 20, about 25, about 30, about 35, about 40, about 45, or about 50 mg. For example, the aminosterol or a salt or derivative thereof can be administered orally and the dose of the aminosterol or a salt or derivative thereof can be escalated in about 25 mg increments.

In the methods of the invention, where the aminosterol or a salt or derivative thereof is formulated for oral administration, the composition can be a liquid, capsule, or tablet designed to disintegrate in either the stomach, upper small intestine, or more distal portions of the intestine.

In another aspect of the invention, the aminosterol or a salt or derivative thereof is administered intranasally and the starting dose of the aminosterol or a salt or derivative thereof ranges from about 0.001 mg to about 3 mg. For example, in this embodiment, where the aminosterol or a salt or derivative thereof is administered intranasally, the starting dose of the aminosterol or a salt or derivative thereof can be about 0.001, about 0.005, about 0.01, about 0.02, about 0.03, about 0.05, about 0.06, about 0.07, about 0.08, about 0.09, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 1.0, about 1.1, about 1.25, about 1.3, about 1.4, about 1.5, about 1.6, about 1.7, about 1.75, about 1.8, about 1.9, about 2.0, about 2.1, about 2.25, about 2.3, about 2.4, about 2.5, about 2.6, about 2.7, about 2.75, about 2.8, about 2.9, or about 3 mg.

In one aspect of the invention, where the aminosterol or a salt or derivative thereof is administered intranasally, the dose of the aminosterol or a salt or derivative thereof for the subject following escalation can be fixed following dose escalation at a range of from about 0.001 mg up to about 6 mg. For example, in methods of the invention where the aminosterol or a salt or derivative thereof is administered intranasally, the dose of the aminosterol or a salt or derivative thereof for the subject following dose escalation can be fixed at about 0.001, about 0.005, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, or about 6 mg.

In yet another embodiment of the invention, where the aminosterol or a salt or derivative thereof is administered intranasally, the dose of the aminosterol or a salt or derivative thereof for the subject following escalation is a dose which is sub-therapeutic when given orally or by injection.

In one embodiment of the invention, where the aminosterol or a salt or derivative thereof is administered intranasally, the dose of the aminosterol or a salt or derivative thereof can be escalated in increments of about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg.

In a further embodiment of the invention, where the aminosterol or a salt or derivative thereof is formulated for intranasal administration, the composition can be a dry powder or nasal spray or liquid nasal spray.

In one aspect of the invention, the dose of the aminosterol or a salt or derivative thereof can be escalated every about 1 to about 14 days. For example, the dose of the aminosterol or a salt or derivative thereof can be escalated every about 3 to 5 days. In another embodiment, the dose of the aminosterol or a salt or derivative thereof is escalated every about 1, about 2, about 3, about 4, about 5, about 6, about 7, about 8, about 9, about 10, about 11, about 12, about 13, or about 14 days. In yet further embodiments, the dose of the aminosterol or a salt or derivative thereof is escalated about 1×/week, about 2×/week, about every other week, or about 1×/month.

In the methods of the invention, the fixed dose of an aminosterol or a salt or derivative thereof can be given for any desired frequency, such as once per day, every other day, once per week, twice per week, three times per week, four times per week, five times per week, six times per week, every other week, or every few days. In one embodiment, the fixed aminosterol dose is given once per day.

The fixed dose of an aminosterol or a salt or derivative thereof can be given for a few weeks, followed by skipping a few weeks, followed by restarting aminosterol treatment. In addition, the fixed aminosterol dose can be incrementally reduced after the fixed dose of aminosterol or a salt or derivative thereof has been administered to the subject for a period of time. For example, the fixed aminosterol dose can be varied plus or minus a defined amount to enable a modest reduction or increase in the fixed dose. In one embodiment, the fixed aminosterol dose is increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

In one aspect of the invention, the subject is suffering from a disorder of gastrointestinal motility. In another aspect of the invention, the subject is suffering from a condition or disorder selected from the group consisting of chronic idiopathic constipation, Irritable bowel syndrome, Opioid-induced constipation, and Inflammatory Bowel Disease. In yet a further aspect of the invention, the subject is suffering from a neurodegenerative disease. For example, the neurodegenerative disease can be Parkinson's Disease, Alzheimer's disease (AD), Huntington's chorea and/or Huntington's disease, Multiple Sclerosis, Amyotorphic Lateral Sclerosis (ALS), multiple system atrophy (MSA), schizophrenia, Friedreich's ataxia, vascular dementia, Lewy Body dementia or disease, spinal muscular atrophy, supranuclear palsy, fronto temperal dementia, progressive nuclear palsy, Guadeloupian Parkinsonism, spinocerebellar ataxia, autism, dementia of aging, neuropathy of diabetes, peripheral sensory neuropathy, cerebral palsy, epilepsy, diabetic neuropathy, traumatic head and/or spine injury, stroke, or depression.

In the methods of the invention, the compound can be an aminosterol that can inhibit the formation of actin stress fibers in endothelial cells stimulated by a ligand known to induce stress fiber formation, having the chemical structure of Formula I:

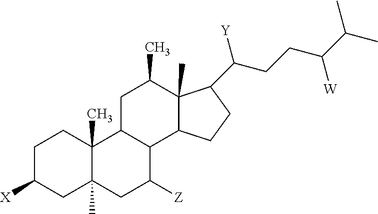

wherein:

W is 24S —OSO₃ or 24R-OSO₃;

X is 3β-H₂N—(CH₂)₄—NH—(CH₂)₃—NH— or 3α-H₂N—(CH₂)₄—NH—(CH₂)₃—NH—;

Y is 20R-CH₃; and

Z is 7α or 7β —OH

In another aspect of the invention, the aminosterol or a salt or derivative thereof is represented by the family of related compounds (compounds 1-8) isolated from Squalus acanthias:

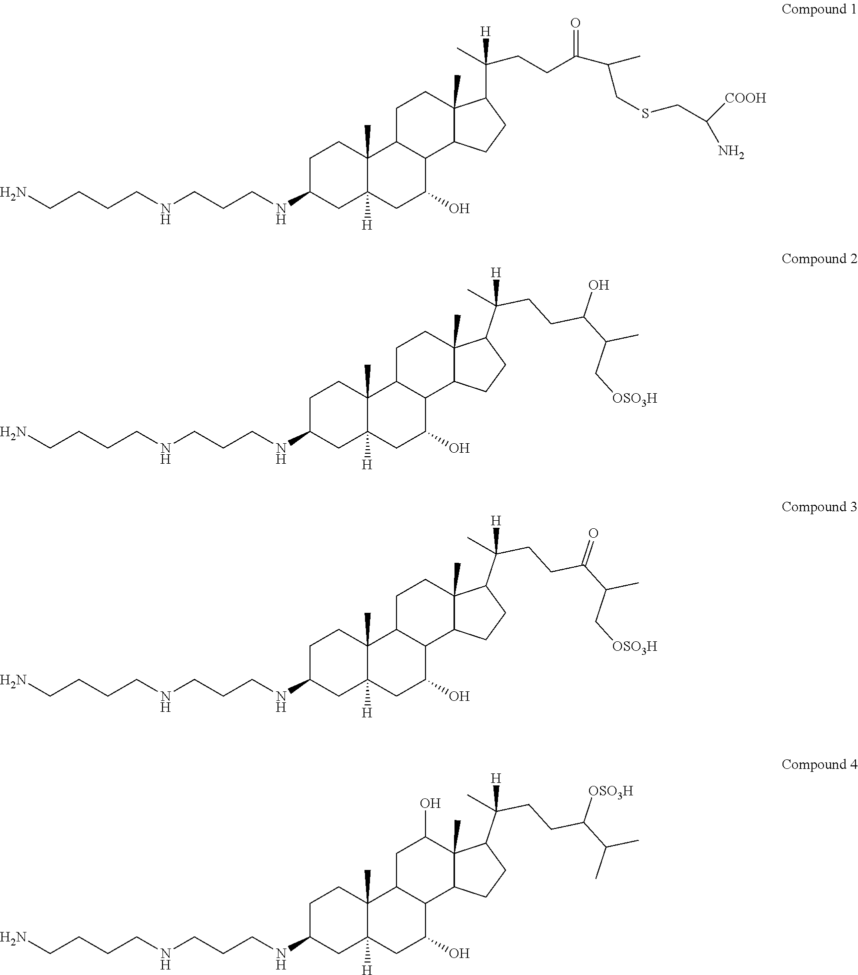

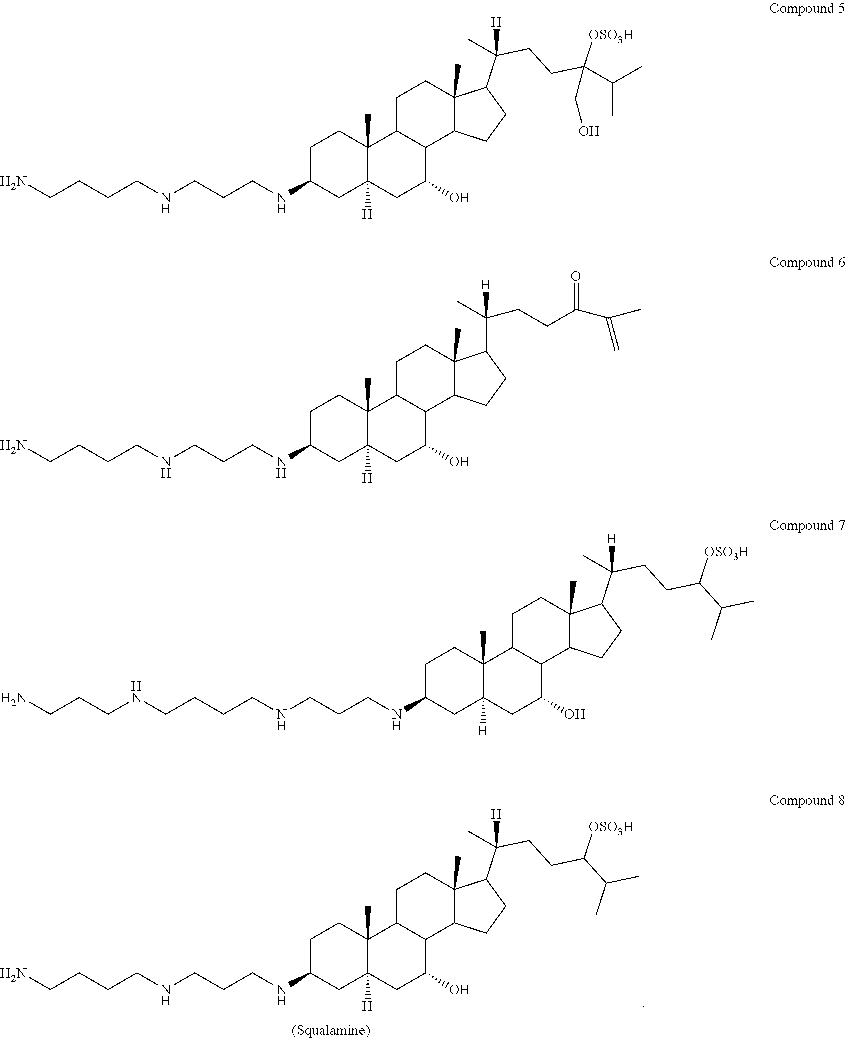

In one aspect of the invention, the aminosterol is Aminosterol 1436 or a pharmaceutically acceptable salt or derivative thereof or squalamine or a pharmaceutically acceptable salt or derivative thereof. The aminosterol can also be a synthetic aminosterol.

In a further aspect of the invention, the aminosterol (a) can comprise a sterol nucleus and a polyamine, attached at any position on the sterol, such that the molecule exhibits a net charge of at least +1, the charge being contributed by the polyamine; or (b) can comprise a bile acid nucleus and a polyamine, attached at any position on the bile acid, such that the molecule exhibits a net charge of at least +1, the charge being contributed by the polyamine; or (c) can be modified to include one or more of the following: (1) substitutions of the sulfate by a sulfonate, phosphate, carboxylate, or other anionic moiety chosen to circumvent metabolic removal of the sulfate moiety and oxidation of the cholesterol side chain; (2) replacement of a hydroxyl group by a non-metabolizable polar substituent, such as a fluorine atom, to prevent its metabolic oxidation or conjugation; and (3) substitution of various ring hydrogen atoms to prevent oxidative or reductive metabolism of the steroid ring system; or (d) is a derivative of squalamine or natural aminosterol modified through medicinal chemistry to improve bio-distribution, ease of administration, metabolic stability, or any combination thereof.

In one embodiment, the aminosterol is administered in combination with at least one additional active agent to achieve either an additive or synergistic effect. For example, the additional active agent can be administered via a method selected from the group consisting of concomitantly, as an admixture, separately and simultaneously or concurrently, or separately and sequentially.

In one embodiment, the aminosterol or a salt or derivative thereof is present in a composition comprising at least one pharmaceutically acceptable carrier.

Finally, in the methods of the invention the subject can be human, including a subject at risk for developing constipation.

Both the foregoing summary of the invention and the following brief description of the drawings and the detailed description of the invention are exemplary and explanatory and are intended to provide further details of the invention as claimed. Other objects, advantages, and novel features will be readily apparent to those skilled in the art from the following detailed description of the invention.

### BRIEF DESCRIPTION OF THE DRAWINGS

FIG. 1 shows a picture of a Bristol Stool Chart, which is a diagnostic medical tool designed to classify the form of human faeces into seven categories.

FIG. 2 shows a Constipation Assessment Scale (CAS), developed by McMillan and Williams (1989). The CAS was based on earlier research and clinical literature and includes eight commonly identified characteristics of constipation

FIG. 3 (panels A and B) shows prokinetic activity of squalamine (ENT-01, a synthetic squalamine salt comprising squalamine as the active ion). As shown in panel A, in Stage 1 (single dose), cumulative prokinetic response rate was defined as the proportion of patients who had a complete spontaneous bowel movements (CSBM) within 24 hours of dosing. In Stage 2 (daily dosing), a prokinetic response was defined as the fraction of patients who had a CSBM within 24 hours of dosing on at least 2 out of 3 days at any given dose. As shown in panel B, the prokinetic dose of squalamine was significantly related to baseline constipation severity (p=0.00055). Patients with baseline CSBM<1 required a higher dose (mean, 192 mg) of squalamine than patients with CSBM≥1 (mean, 120 mg).

FIG. 4 is a schematic (flowchart) showing patient disposition in Stage 2. (1) Patients first enrolled (n=40); (2) 6 patients failed to meet dosing criteria and were excluded; (3) 34 patients were dosed; (4) 5 patients were discontinued; 3 patients withdrew consent (with 1 patient lost to follow up and 2 patients withdrew because of diarrhea); and 2 patients discontinued because of an adverse event (recurrent dizziness after medication); (5) 31 patients had an assessable prokinetic response; and (6) 29 patients completed dosing.

### DETAILED DESCRIPTION

### I. Overview

The present invention is directed to methods of treating constipation and constipation related disorders.

In a first aspect, described is a method of treating, preventing, and/or slowing the onset or progression of constipation and/or a constipation-related symptom in a subject in need comprising administering to the subject a therapeutically effective amount of at least one aminosterol, or a salt or derivative thereof, provided that the administering does not comprise oral administration. For example, the method can comprise administration selected from nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof. In one aspect, administering comprises nasal administration.

The therapeutically effective amount of the at least one aminosterol or a salt or derivative thereof: (a) comprises about 0.1 to about 20 mg/kg body weight of the subject; and/or (b) comprises about 0.1 to about 15 mg/kg body weight of the subject; and/or (c) comprises about 0.1 to about 10 mg/kg body weight of the subject; and/or (d) comprises about 0.1 to about 5 mg/kg body weight of the subject; and/or (e) comprises about 0.1 to about 2.5 mg/kg body weight of the subject; and/or (f) comprises about 0.001 to about 500 mg/day; and/or (g) comprises about 0.001 to about 250 mg/day; and/or (h) comprises about 0.001 to about 125 mg/day; and/or (i) comprises about 0.001 to about 50 mg/day; and/or (j) comprises about 0.001 to about 25 mg/day; and/or (k) comprises about 0.001 to about 10 mg/day; and/or (l) comprises nasal administration and wherein the therapeutically effective amount of the at least one aminosterol, or a salt or derivative thereof comprises about 0.001 to about 6 mg/day; and/or (m) comprises nasal administration and wherein the therapeutically effective amount of the at least one aminosterol, or a salt or derivative thereof comprises about 0.001 to about 4 mg/day; and/or (n) comprises nasal administration and wherein the therapeutically effective amount of the at least one aminosterol, or a salt or derivative thereof comprises about 0.001 to about 2 mg/day; and/or (o) comprises nasal administration and wherein the therapeutically effective amount of the at least one aminosterol, or a salt or derivative thereof comprises about 0.001 to about 1 mg/day. Other exemplary dosages are described herein.

In a second aspect, the method comprises the following steps: (i) identifying a starting aminosterol dose for a subject; (ii) administering an escalating dose of the aminosterol over a period of time until an effective aminosterol dose is identified, wherein the effective aminosterol dose is the dose where improvement or resolution of constipation and/or a constipation-related symptom is observed, and (iii) fixing the aminosterol dose at that level in that particular subject.

As described in Example 1, a study was conducted in patients with Parkinson's disease (PD). PD is a progressive neurodegenerative disorder caused by accumulation of the protein α-synuclein (αS) within the enteric nervous system (ENS), autonomic nerves and brain. While the study described herein assessed patients with PD, symptoms assessed and contemplated to be resolved by aminosterol treatment, such as constipation, are not restored by the replacement of dopamine and are thus not unique to PD but rather common across a variety of disorders which involve impaired function of neural pathways, referred to herein as “brain-gut” disorders. Constipation is a symptom that results from impaired function of neural pathways not restored by replacement of dopamine.

The methods and compositions disclosed herein permit exerting pharmacological control over the ENS in a manner that is without precedent in the literature. While the study described herein assessed the effectiveness of treating PD-related constipation, it is contemplated that the effect of aminosterols on the ENS can broadly treat a variety of constipation conditions, symptoms, and constipation associated disorders.

Most surprisingly, as described in Example 1, it was discovered that aminosterol dosing is patient specific, as the dose is likely related to the extent of neuronal damage, with greater neuronal damage correlating with the need for a higher aminosterol dose to obtain a desired therapeutic result. This was not known prior to the present invention. Thus, one aspect of the present invention is directed to methods of treating constipation or constipation-related symptoms in a subject, where the method comprises determining an effective therapeutic aminosterol dose for the subject. In addition, it was also surprisingly discovered that the starting aminosterol dose is dependent upon the severity of the constipation or constipation-related symptom. Specifically, if the constipation or constipation-related symptom is severe, then the starting aminosterol dose, prior to dose escalation, should be higher than if the constipation or constipation-related symptom is moderate. “Severe” constipation can be defined as less than one complete spontaneous bowel movement (CSBM) or spontaneous bowel movement (SBM) per week.

One impact of the present invention is that recognizing that an aminosterol dose useful in treating constipation or constipation-related symptoms is patient specific can prevent the use of incorrect aminosterol doses for patients. This is a significant discovery, as if a subject is put on an aminosterol dose that is too high, then resultant nausea, vomiting, and abdominal discomfort can result in the patient going off the drug, with the constipation and constipation-related symptoms remaining untreated. Similarly, if a subject is put on an aminosterol dose that is too low, then the constipation and/or constipation-related symptoms will not be successfully treated. Prior to the present invention, there was no recognition that aminosterol doses useful in treating constipation and/or constipation-related symptoms had no relation to the sex, age, weight, ethnicity, or other similar patient characteristics. This is unexpected, as it is contrary to dosing strategies for almost all other medications.

Constipation, which is generally described as having fewer than three bowel movements a week, is the most common chronic gastrointestinal disorder in adults. In the general population rates of constipation are 2-30%. Among elderly people living in a care home the rate of constipation is 50-75%. Further, (i) 12% of the population worldwide reports having constipation (Wald et al. 2017); (ii) chronic constipation accounts for 3% of all visits annually to pediatric outpatient clinics (Walia et al. 2009); (iii) constipation-related health care costs total $6.9 billion in the US annually (Locke et al. 2000); (iv) more than four million Americans have frequent constipation, accounting for 2.5 million physician visits a year; and (v) around $725 million is spent on laxative products each year in America. Thus, constipation is a serious medical issue having a significant impact on medical resources as well as having significant deleterious effects on a large number of subjects.

Not to be bound by theory, it is believed that aminosterols target neurotoxic aggregates of αS in the gastrointestinal tract, and restore function of the enteric nerve cells. The now-functional enteric nerve cells prevent retrograde trafficking of proteins, such as alpha-synuclein, to the brain, resulting in restoring gastrointestinal function.

Constipation serves as an early indicator of many neurodiseases such as PD to the extent that it is suspected to correlate with the formation of toxic αS aggregates within the enteric nervous system (ENS) (Braak et al. 2003). As a result of the normal trafficking of αS aggregates from the ENS to the central nervous system (CNS) via afferent nerves such as the vagus (Holmqvist et al. 2014; Svensson et al. 2015), neurotoxic aggregates accumulate progressively within the brainstem and more rostral structures. Inhibiting αS aggregation in the ENS may, thus, reduce the continuing neuro disease process in both the ENS and CNS (Phillips et al. 2008). This relationship between the ENS and CNS is sometimes described herein as “brain-gut” in relation to a class of disorders or the axis of aminosterol activity.

Not to be bound by theory, based on the data described herein, it is believed that aminosterols improve bowel function by acting locally on the gastrointestinal tract, as supported by the oral bioavailability <0.3%. An orally administered aminosterol such as squalamine, the active ion of ENT-01, stimulates gastro-intestinal motility in mice with constipation due to overexpression of human αS (West et al, manuscript in preparation). Perfusion of an aminosterol such as squalamine through the lumen of an isolated segment of the bowel from the PD mouse model results in excitation of IPANs (intrinsic primary afferent neuron), the major sensory neurons of the ENS that communicate with the myenteric plexus, increasing the frequency of propulsive peristaltic contractions and augmenting neural signals projecting to the afferent arm of the vagus.

Systemic absorption of the aminosterol following oral administration was negligible both in this study and in prior studies involving mice, rats and dogs. Prior studies demonstrated that intravenous administration of squalamine was not associated with increased gastrointestinal motility, despite reaching systemic blood levels one thousand-fold greater than that achieved by orally administered squalamine. These data suggest that the effect is mediated by local action in the GI tract. The topical action would also explain why adverse events were largely confined to the gastrointestinal tract.

Several exploratory endpoints were incorporated into the trial described in Example 1 to evaluate the impact of an aminosterol on neurologic symptoms associated with a neurodisease such as PD. Following aminosterol treatment, the Unified Parkinson's Disease Rating Scale (UPDRS) score, a global assessment of motor and non-motor symptoms, showed significant improvement. In particular, constipation, which was used as a marker for measuring impact and effectiveness of the aminosterol on the tested patient population, showed dramatic improvement. Interestingly, most indices related to bowel function returned to baseline value by the end of the 2-week wash-out period, i.e., in the absence of study drug, while improvement in the CNS symptoms persisted. The rapid improvement in certain CNS symptoms is consistent with a mechanism whereby nerve impulses initiated from the ENS following aminosterol administration augment afferent neural signaling to the CNS. This may stimulate the clearance of αS aggregates within the afferent neurons themselves as well as the secondary and tertiary neurons projecting rostrally within the CNS, since it is known that neural stimulation is accompanied by increased neuronal autophagic activity (Shehata et al. 2012). It is believed that after cessation of aminosterol administration, the neurons of the CNS gradually re-accumulate an αS burden either locally or via trafficking from αS re-aggregation within the gut.

Low bioavailability: As described in Example 1, in preclinical studies, squalamine (ENT-01) exhibited an oral bioavailability of about 0.1% in both rats and dogs. In Stage 1 of the phase 2 study, oral dosing up to 200 mg (114 mg/m²) yielded an approximate oral bioavailability of about 0.1%, based on a comparison of a pharmacokinetic data of the oral dosing and the pharmacokinetic data measured during prior phase 1 studies of IV administration of squalamine. Thus, in one embodiment of the invention, aminosterol dosing, either oral or intranasal, results in a bioavailability of less than about 3%, less than about 2.5%, less than about 2%, less than about 1.5%, less than about 1%, less than about 0.9%, less than about 0.8%, less than about 0.7%, less than about 0.6%, less than about 0.5%, less than about 0.4%, less than about 0.3%, less than about 0.2%, or about 0.1% or less.

### II. Methods of Determining Compositions Comprising a “Fixed Dose” of Aminosterol

In one aspect, the disclosure relates to the surprising discovery of a method to determine a “fixed dose” of an aminosterol composition for treating constipation and/or constipation-related symptoms in a subject that is not age, size, or weight dependent but rather is individually calibrated. The “fixed dose” obtained through this method yields highly effective results in treating constipation.

A. “Fixed Aminosterol Dose”

A “fixed aminosterol dose,” also referred to herein as a “fixed escalated aminosterol dose,” which will be therapeutically effective, is determined for each subject by establishing a starting dose of an aminosterol composition and a threshold for improvement of constipation or a constipation-related symptom. Following determining a starting aminosterol dose for a particular subject, the aminosterol dose is then progressively escalated by a consistent amount over consistent time intervals until the desired improvement in constipation or a constipation-related symptom is achieved; this aminosterol dosage is the “fixed escalated aminosterol dosage” for that particular subject for constipation.

This therapeutically effective “fixed dose” is then maintained throughout treatment and/or prevention. Thus, even if the subject goes “off drug” and ceases taking the aminosterol composition, the same “fixed dose” is taken with no ramp up period following re-initiation of aminosterol treatment for constipation.

Not to be bound by theory, it is believed that the aminosterol dose is dependent on the severity of nerve damage relating to constipation, e.g. the dose may be related to the extent of nervous system damage in the subject's gut.

The aminosterol can be administered via any pharmaceutically acceptable means, such as by injection (e.g., IM, IV, or IP), oral, pulmonary, intranasal, etc. Preferably, the aminosterol is administered orally, intranasally, or a combination thereof.

Oral dosage of an aminosterol can range from about 1 to about 500 mg/day, or any amount in-between these two values. Other exemplary dosages of orally administered aminosterols include, but are not limited to, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, or about 500 mg/day.

Intranasal dosages of an aminosterol are much lower than oral dosages of an aminosterol. Examples of such intranasal aminosterol low dosages include, but are not limited to, about 0.001 to about 6 mg, or any amount in-between these two values. For example, the low dosage of an intranasal administered aminosterol can be about 0.001, about 0.005, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, or about 6 mg/day.

For intranasal (IN) administration, it is contemplated that the aminosterol dosage may be selected such that it would not provide any pharmacological effect if administered by any other route and, in addition, does not result in negative effects. For example, Aminosterol 1436 is known to have the pharmacological effects of a reduction in food intake and weight loss. Therefore, in the IN methods of the invention, if the aminosterol is Aminosterol 1436 or a salt or derivative thereof, then if the IN Amino sterol 1436 dosage is administered via another route, such as oral, IP, or IV, then the Aminosterol 1436 dosage will not result in a noticeable reduction in food intake or noticeable weight loss. Similarly, squalamine is known to produce the pharmacological effects of nausea, vomiting and/or reduced blood pressure. Thus, in the IN methods of the invention, if the aminosterol is squalamine or a salt or derivative thereof, then if the IN squalamine dosage is administered via another route, such as oral, IP, or IV, then the squalamine dosage will not result in noticeable nausea, vomiting, and/or a reduction in blood pressure.

Dose escalation: When determining a “fixed aminosterol dosage” for a particular subject, a subject is started at a lower dose and then the dose is escalated until a positive result is observed for constipation or a constipation-related symptom. For example, determination of the fixed aminosterol dosage for treating constipation is shown in Example 1. Aminosterol doses can also be de-escalated (reduced) if any given aminosterol dose induces a persistent undesirable side effect, such as diarrhea, vomiting, or nausea.

The starting aminosterol dose is dependent on the severity of the symptom—e.g. for a subject experiencing severe constipation, defined as less than one complete spontaneous bowel movement (CSBM) or spontaneous bowel movement (SBM) a week, the starting oral aminosterol dose can be about 150 mg or greater. In contrast, for a subject having moderate constipation, e.g., defined as having one or more CSBM or SBM per week, the starting aminosterol dose can be about 75 mg or less. Thus, as an example, a subject experiencing moderate constipation can be started at an aminosterol dosage of about 75 mg/day, whereas a subject experiencing severe constipation can be started at an aminosterol dosage of about 150 mg/day.

In other embodiments, a subject experiencing moderate constipation symptoms can be started at an oral aminosterol dosage of from about 10 mg/day to about 75 mg/day, or any amount in-between these values. The moderate symptom may be moderate constipation based on the rate of CSBM or SBM per week. For example, starting oral aminosterol dosage for patients with moderate to mild constipation can be about 1, about 5, about 10, about 15, about 20, about 25, about 30, about 35, about 40, about 45, about 50, about 55, about 60, about 65, about 70, up to less than or equal to about 75 mg/day. A fixed escalated oral aminosterol dose for a mild or moderately constipated patient is likely to range from about 5 mg up to about 350 mg, or any amount in-between these two values as described herein. In some embodiments, an oral fixed aminosterol dose, following dose escalation, is from about 50 to about 300 mg/daily, or from about 75 to about 275 mg/daily.

In yet further embodiments, when the subject is experiencing severe constipation symptoms, as for example defined by the subject experiencing less than one CSBM or SBM per week, the subject can be started at an oral aminosterol dosage ranging from about 75 to about 300 mg/day, or any amount in-between these two values. In other embodiments, the starting oral aminosterol dosage for severely constipated patients can be, for example, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, or about 300 mg/day. A “fixed escalated” oral aminosterol dose for a severely constipated patient is likely to range from about 75 mg up to about 500 mg. As described in Example 1, a positive effect was defined as a dose that resulted in a complete spontaneous bowel movement (CSBM) within 24 hours of dosing on at least 2 of 3 days at a given dose. In some embodiments, the desired rate of CSBM in the subject is one or more CSBM per week, 2 or more CSBM per week, or 3 or more CSBM per week.

In some embodiments, the starting aminosterol dose may be about 125 mg or about 175 mg; again dependent on the severity of the symptom, such as constipation.

Starting IN aminosterol dosages prior to dose escalation can be, for example, about 0.001 mg to about 3 mg, or any amount in-between these two values. For example, the starting aminosterol dosage for IN administration, prior to dose escalation, can be, for example, about 0.001, about 0.005, about 0.01, about 0.02, about 0.03, about 0.05, about 0.06, about 0.07, about 0.08, about 0.09, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 1.0, about 1.1, about 1.25, about 1.3, about 1.4, about 1.5, about 1.6, about 1.7, about 1.75, about 1.8, about 1.9, about 2.0, about 2.1, about 2.25, about 2.3, about 2.4, about 2.5, about 2.6, about 2.7, about 2.75, about 2.8, about 2.9, or about 3 mg.

In exemplary embodiments, the fixed dose of the aminosterol is given periodically as needed. For example, the fixed aminosterol dose can be given once per day. The aminosterol dose can also be given every other day, 2, 3, 4, 5 or 6× per week, once/week, or 2×/week. In another embodiment, the aminosterol dose can be given every other week, or it can be given for a few weeks, followed by skipping a few weeks (as the effects persist following treatment), followed by restarting aminosterol treatment.

When calculating a fixed escalated aminosterol dose, the dose can be escalated following any suitable time period. In one embodiment, the aminosterol dose is escalated every about 3 to about 7 days by about a defined amount until a desired improvement is reached. In one embodiment, the aminosterol dose is escalated every about 3 to 5 days until a desired improvement is reached. For example, when the symptom being treated/measured is constipation, threshold improvement can be an increase of one CSBM or SBM per week or at least a total of three bowel movements per week. In other embodiments, the aminosterol dose can be escalated every about 1, about 2, about 3, about 4, about 5, about 6, about 7, about 8, about 9, about 10, about 11, about 12, about 13, or about 14 days. In other embodiments, the aminosterol dose can be escalated about 1×/week, about 2×/week, about every other week, or about 1×/month.

During dose escalation, the aminosterol dosage can be increased by a defined amount. For example, when the aminosterol is administered orally, the dose can be escalated in increments of about 5, about 10, about 15, about 20, about 25, about 30, about 35, about 40, about 45, or by about 50 mg. When the aminosterol is administered intranasally, then the dosage can be increased in increments of about, for example, about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg.

In exemplary embodiments, an orally administered aminosterol dose is escalated every about 3 to about 5 days by about 25 mg until the desired improvement or resolution of constipation is reached.

In another embodiment, a fixed dose of an aminosterol can be varied plus or minus a defined amount to enable a modest reduction in a dose to eliminate adverse events, or a modest increase in a dose if clinical results suggest this is desirable—e.g., no or minimal adverse events and potential increased efficacy with a modest increase in dose. For example, in one embodiment a fixed aminosterol dose can be increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

B. Constipation or Constipation-Related Symptoms to be Evaluated

The “fixed” dose of an aminosterol or a salt or derivative thereof is determined based upon the effect an escalated aminosterol dose has, over a period of time, on constipation or a constipation-related symptom. Measurable constipation-related symptoms that can be evaluated include, for example: (1) frequency of constipation; (2) duration of constipation symptoms; (3) frequency of bowel movements; (4) fecal incontinence/encopresis; (5) abdominal pain; (6) abdominal distension or bloating; (7) abdominal discomfort; (8) stomach cramps; (9) stool consistency; (10) painful defecation/rectal pain with bowel movement; (11) rectal burning during or after bowel movement; (12) rectal bleeding or tearing during or after a bowel movement; (13) ease of defecation/passing stool; (14) straining during defecation and/or straining or squeezing to try to pass bowel movements; (15) incomplete evacuation or bowel movement; (16) unsuccessful attempts at evacuation; (17) sensation of incomplete bowel evacuation; (18) sensation of anorectal obstruction/blockage; (19) bowel movements that were too hard; (20) bowel movements that were too small, (21) change in amount of gas passed rectally; (22) less frequent bowel movements; (23) oozing liquid stool; (24) rectal fullness or pressure; (25) small stool size; (26) urge but inability to pass stool; and/or (27) personal judgement of constipation.

The symptoms can be measured using a clinically recognized scale or tool, as detailed herein. For example, a symptom evaluated or measured can be the number of CSBM or SMB per week, with an increase in CSBM or SBM equating with an improvement in constipation. “Resolving” constipation for an adult can be a result in a subject following treatment of 3 or more CSMB or SBM per week, which is considered normal for an adult.

C. Aminosterols

U.S. Pat. No. 6,962,909, entitled “Treatment of neovascularization disorders with squalamine,” discloses various aminosterols, and this disclosure is specifically incorporated by reference with respect to its teaching of aminosterol compounds. Any aminosterol known in the art, including those described in U.S. Pat. No. 6,962,909, can be used in the disclosed compositions. In some embodiments, the aminosterol present in the compositions of the invention is Aminosterol 1436 or a salt or derivative thereof, squalamine or a salt or derivative thereof, or a combination thereof.

An aminosterol such as squalamine (ENT-01 in the examples) inhibits the formation of αS aggregates in vitro and in vivo, reverses motor dysfunction in the C. elegans αS model, and restores gastrointestinal motility in mouse models of PD.

Squalamine (ENT-01) has limited bioavailability in rats and dogs. Based on measurement of portal blood concentrations following oral dosing of radioactive ENT-01 to rat's absorption of ENT-01 from the intestine is low. As a consequence, the principal focus of safety is on local effects on the gastrointestinal tract. However, squalamine (ENT-01) appears to be well tolerated in both rats and dogs.

For instance, useful aminosterol compounds comprise a bile acid nucleus and a polyamine, attached at any position on the bile acid, such that the molecule exhibits a net positive charge contributed by the polyamine.

Thus, in some embodiments, the disclosed methods comprise administering a therapeutically effective amount of one or more aminosterols having the chemical structure of Formula I:

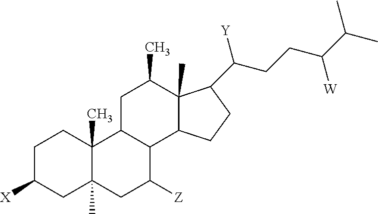

wherein,

W is 24S —OSO₃ or 24R-OSO₃;

X is 3β-H₂N—(CH₂)₄—NH—(CH₂)₃—NH— or 3α-H₂N—(CH₂)₄—NH—(CH₂)₃—NH—;

Y is 20R-CH₃; and

Z is 7α or 7β —OH.

In another embodiment of the invention, the aminosterol is one of the naturally occurring aminosterols (1-8) isolated from Squalus acanthias:

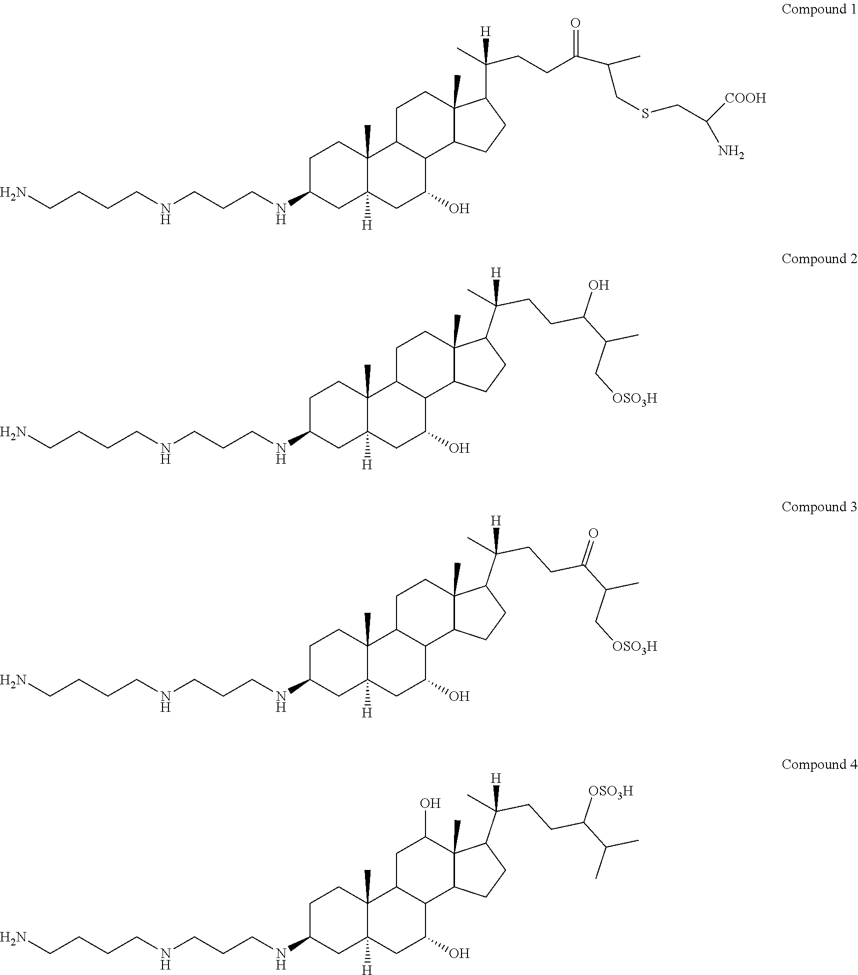

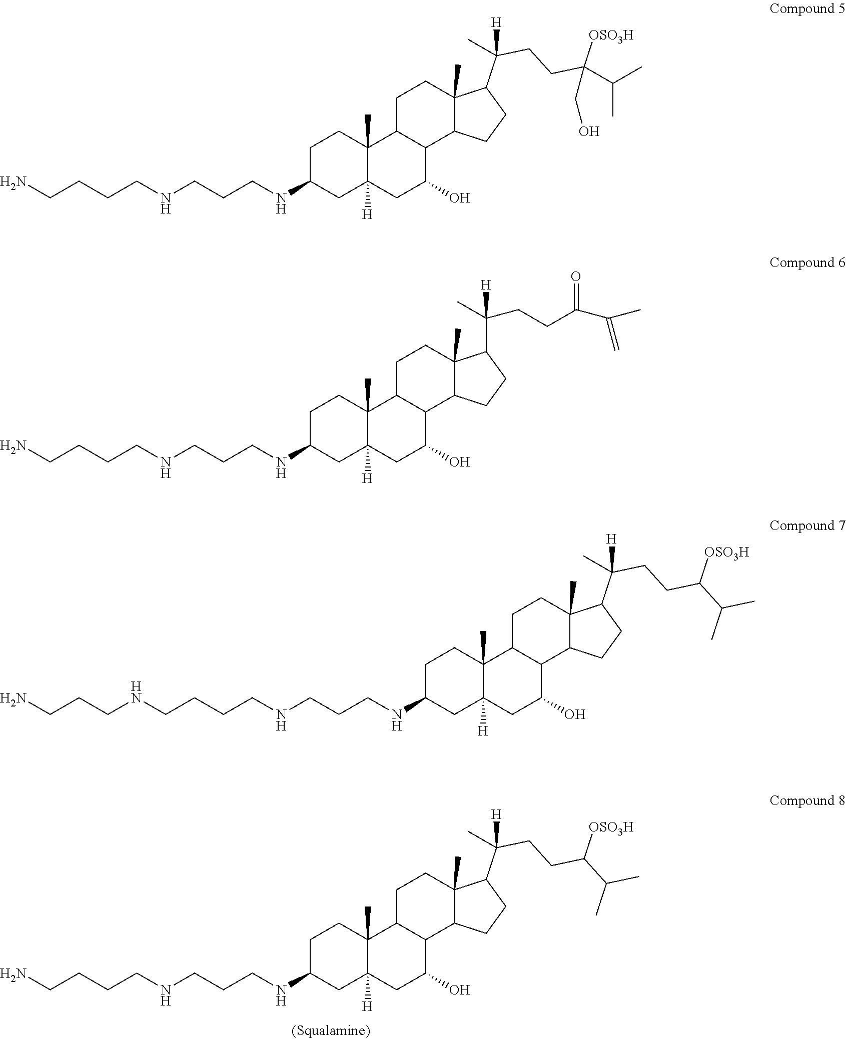

Variants or derivatives of known aminosterols, such as squalamine, Aminosterol 1436, or an aminosterol isolated from Squalus acanthias, may be used in the disclosed compositions and methods.

In one embodiment, the aminosterol is Aminosterol 1436 or a squalamine isomer. In yet another embodiment of the invention, the aminosterol is a derivative of squalamine or another naturally occurring aminosterol modified through medical chemistry to improve biodistribution, ease of administration, metabolic stability, or any combination thereof. In another embodiment, the squalamine or aminosterol is modified to include one or more of the following: (1) substitutions of the sulfate by a sulfonate, phosphate, carboxylate, or other anionic moiety chosen to circumvent metabolic removal of the sulfate moiety and oxidation of the cholesterol side chain; (2) replacement of a hydroxyl group by a non-metabolizable polar substituent, such as a fluorine atom, to prevent its metabolic oxidation or conjugation; and (3) substitution of various ring hydrogen atoms to prevent oxidative or reductive metabolism of the steroid ring system.

In yet another embodiment, the aminosterol comprises a sterol nucleus and a polyamine, attached at any position on the sterol, such that the molecule exhibits a net charge of at least +1, the charge being contributed by the polyamine.

In yet another embodiment, the aminosterol comprises a bile acid nucleus and a polyamine, attached at any position on the bile acid, such that the molecule exhibits a net positive charge being contributed by the polyamine.

In some embodiments, the compositions used in the methods of the invention comprise: (a) at least one pharmaceutical grade aminosterol; and optionally (b) at least one phosphate selected from the group consisting of an inorganic phosphate, an inorganic pyrophosphate, and an organic phosphate. In some embodiments, the aminosterol is formulated as a weakly water soluble salt of the phosphate. In some embodiments, the phosphate is an inorganic polyphosphate, and the number of phosphates can range from about 3 (tripolyphosphate) to about 400, or any number in-between these two values. In other embodiments, the phosphate is an organic phosphate which comprises glycerol 2 phosphates.

In some embodiments, the aminosterol is selected from the group consisting of: (a) squalamine or a pharmaceutically acceptable salt or derivative thereof; (b) a squalamine isomer; (c) a squalamine phosphate salt; (c) Aminosterol 1436 or a pharmaceutically acceptable salt or derivative thereof; (e) an aminosterol 1436 isomer; (f) an aminosterol 1436 phosphate salt; (g) an aminosterol comprising a sterol or bile acid nucleus and a polyamine, attached at any position on the sterol or bile acid, such that the molecule exhibits a net charge of at least +1, the charge being contributed by the polyamine; (h) an aminosterol which is a derivative of squalamine modified through medical chemistry to improve biodistribution, ease of administration, metabolic stability, or any combination thereof; (i) an aminosterol modified to include one or more of the following: (i) substitutions of the sulfate by a sulfonate, phosphate, carboxylate, or other anionic moiety chosen to circumvent metabolic removal of the sulfate moiety and oxidation of the cholesterol side chain; (ii) replacement of a hydroxyl group by a non-metabolizable polar substituent, such as a fluorine atom, to prevent its metabolic oxidation or conjugation; and (iii) substitution of various ring hydrogen atoms to prevent oxidative or reductive metabolism of the steroid ring system; (j) an aminosterol that can inhibit the formation of actin stress fibers in endothelial cells stimulated by a ligand known to induce stress fiber formation, having the chemical structure of Formula I (above); (k) a synthetic aminosterol; or (l) any combination thereof.

In some embodiments, the methods of the invention can employ a formulation of squalamine or Aminosterol 1436 as an insoluble salt of phosphate, polyphosphate, or an organic phosphate ester.

Any pharmaceutically acceptable salt of an aminosterol can be used in the compositions and methods of the invention. For example, a phosphate salt or buffer, free base, succinate, phosphate, mesylate or other salt form associated with low mucosal irritation can be utilized in the methods and compositions of the invention.

D. Routes of Administration

In a first aspect, a method of treating and/or preventing constipation is described, wherein administration is limited to non-oral administration.

In a second aspect, where the method requires first determining a “fixed dose” of an aminosterol, it is appreciated that the “fixed dose” disclosed herein can be administered via any suitable route of administration, including but not limited to oral or intranasal delivery, injection (IP, IV, or IM), or a combination thereof.

Further, co-administration of an aminosterol dose with injectable (e.g., 1P, IV, IM) aminosterol formulations is also contemplated herein. For injectable dosage forms, the dosage form can comprise an aminosterol at a dosage of, for example, about 0.1 to about 20 mg/kg body weight. In other embodiments, the effective daily dosing amount is about 0.1, about 0.5, about 1, about 2, about 3, about 4, about 5, about 6, about 7, about 8, about 9, about 10, about 11, about 12, about 13, about 14, about 15, about 16, about 17, about 18, about 19, or about 20 mg/kg body weight.

The invention also encompasses methods of treatment using a combination of an aminosterol composition administered via one route, e.g., oral, with a second aminosterol composition, comprising the same or a different aminosterol, administered via a different route, e.g., intranasal. For example, in a method of the invention, squalamine can be administered orally and aminosterol 1436 can be administered IN.

E. Dosing Period

The pharmaceutical composition comprising an aminosterol or a derivative or salt thereof can be administered for any suitable period of time, including as a maintenance dose for a prolonged period of time. Dosing can be done on an as needed basis using any pharmaceutically acceptable dosing regimen. Aminosterol dosing can be no more than 1× per day, once every other day, once every three days, once every four days, once every five days, once every six days, once a week, or divided over multiple time periods during a given day (e.g., twice daily).

In other embodiments, the composition can be administered: (1) as a single dose, or as multiple doses over a period of time; (2) at a maintenance dose for an indefinite period of time; (3) once, twice or multiple times; (4) daily, every other day, every 3 days, weekly, or monthly; (5) for a period of time such as about 1, about 2, about 3, or about 4 weeks, about 1, about 2, about 3, about 4, about 5, about 6, about 7, about 8, about 9, about 10, about 11, or about 12 months, about 1 year, about 1.5 years, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, or about 25 years, or (6) any combination of these parameters, such as daily administration for 6 months, weekly administration for 1 or more years, etc.

Yet another exemplary dosing regimen includes periodic dosing, where an effective dose can be delivered once every about 1, about 2, about 3, about 4, about 5, about 6 days, or once weekly.

In a preferred embodiment, the aminosterol dose is taken in the morning, i.e. on an empty stomach preferably within about two hours of waking up and may be followed by a period without food, such as for example about 60 to about 90 minutes. In other embodiments, the aminosterol dose is taken within about 15 min, about 30 min, about 45 min, about 1 hr, about 1.25 hrs, about 1.5 hrs, about 1.75 hrs, about 2 hrs, about 2.25 hrs, about 2.5 hrs, about 2.75 hrs, about 3 hrs, about 3.25 hrs, about 3.5 hrs, about 3.75 hrs, or about 4 hrs within waking up. In yet further embodiments, the aminosterol dose is followed by about period without food, wherein the period is at least about 30 min, about 45 mins, about 60 mins, about 1.25 hrs, about 1.5 hrs, about 1.75 hrs, or about 2 hrs.

Not to be bound by theory, it is believed that since aminosterols have an impact on circadian rhythms, likely due to ENS signaling thereof, taking the aminosterol dose in the morning enables the synchronization of all the autonomic physiological functions occurring during the day. In other embodiments of the invention, the aminosterol dosage is taken within about 15 mins, about 30 mins, about 45 mins, about 1 hour, about 1.25 hrs, about 1.5 hrs, about 1.75 hrs, about 2 hrs, about 2.25 hrs, about 2.5 hrs, about 2.75 hrs, about 3 hrs, about 3.25 hrs, about 3.5 hrs, about 3.75 hrs, or about 4 hrs of waking up. In addition, in other embodiments of the invention, following the aminosterol dosage the subject has a period of about 15 mins, about 30 mins, about 45 mins, about 1 hours, about 1.25 hrs, about 1.5 hrs, about 1.75 hrs, about 2 hrs, about 2.25 hrs, about 2.5 hrs, about 2.75 hrs, or about 3 hours without food.

F. Composition Components

In some embodiments, a pharmaceutical composition disclosed herein comprises one or more pharmaceutically acceptable carriers, such as an aqueous carrier, buffer, and/or diluent.

In some embodiments, a pharmaceutical composition disclosed herein further comprises a simple polyol compound, such as glycerin. Other examples of polyol compounds include sugar alcohols. In some embodiments, a pharmaceutical composition disclosed herein comprises an aqueous carrier and glycerin at about a 2:1 ratio.

The formulations may conveniently be presented in unit dosage form and may be prepared by any of the methods well known in the art of pharmacy. An exemplary oral dosage form is a tablet or capsule. An exemplary intranasal dosage form is a liquid or powder nasal spray. A nasal spray is designed to deliver drug to the upper nasal cavity, and can be a liquid or powder formulation, and in a dosage form such as an aerosol, liquid spray, or powder.

The aminosterol may be combined or coordinately administered with a suitable carrier or vehicle depending on the route of administration. As used herein, the term “carrier” means a pharmaceutically acceptable solid or liquid filler, diluent or encapsulating material. A water-containing liquid carrier can comprise pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories can be found in the U.S. Pharmacopeia National Formulary, 1857-1859, and (1990). Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art. Examples of filling agents include lactose monohydrate, lactose anhydrous, and various starches; examples of binding agents include various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil® 200, talc, stearic acid, magnesium stearate, calcium stearate, and silica gel. Examples of sweeteners may include any natural or artificial sweetener, such as sucrose, xylitol, sodium saccharin, cyclamate, aspartame, and acesulfame. Examples of flavoring agents are Magnasweet® (trademark of MAFCO), bubble gum flavor, and fruit flavors, and the like. Examples of preservatives include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Any pharmaceutical used for therapeutic administration can be sterile. Sterility is readily accomplished by for example filtration through sterile filtration membranes (e.g., 0.2 micron membranes). Any pharmaceutically acceptable sterility method can be used in the compositions of the invention.

The pharmaceutical composition comprising an aminosterol derivatives or salts thereof will be formulated and dosed in a fashion consistent with good medical practice, taking into account the clinical condition of the individual patient, the method of administration, the scheduling of administration, and other factors known to practitioners.

G. Kits

Aminosterol formulations or compositions of the invention may be packaged together with, or included in a kit along with instructions or a package insert. Such instructions or package inserts may address recommended storage conditions, such as time, temperature and light, taking into account the shelf-life of the aminosterol or derivatives or salts thereof. Such instructions or package inserts may also address the particular advantages of the aminosterol or derivatives or salts thereof, such as the ease of storage for formulations that may require use in the field, outside of controlled hospital, clinic or office conditions.

The invention also provides a pharmaceutical pack or kit comprising one or more containers filled with one or more aminosterol pharmaceutical compositions disclosed herein. The kits may include, for instance, containers filled with an appropriate amount of an aminosterol pharmaceutical composition, either as a powder, a tablet, to be dissolved, or as a sterile solution. Associated with such container(s) can be a notice in the form prescribed by a governmental agency regulating the manufacture, use or sale of pharmaceuticals or biological products, which notice reflects approval by the agency of manufacture, use or sale for human administration. In addition, the aminosterol or a derivative or salt thereof may be employed in conjunction with other therapeutic compounds.

In other aspects, a kit comprising a nasal spray device as described herein is disclosed. In one aspect, the kit may comprise one or more devices as disclosed herein, comprising a disclosed low dose aminosterol composition, wherein the device is sealed within a container sufficient to protect the device from atmospheric influences. The container may be, for example, a foil, or plastic pouch, particularly a foil pouch, or heat sealed foil pouch. Suitable containers sufficient to adequately protect the device will be readily appreciated by one of skill in the art.

In one aspect, the kit may comprise one or more devices as disclosed herein, wherein the device may be sealed within a first protective packaging, or a second protective packaging, or a third protective packaging, that protects the physical integrity of the product. One or more of the first, second, or third protective packaging may comprise a foil pouch. The kit may further comprise instructions for use of the device. In one aspect, the kit contains two or more devices.

In one aspect, the kit may comprise a device as disclosed herein, and may further comprise instructions for use. In one aspect, the instructions may comprise visual aid/pictorial and/or written directions to an administrator of the device.

### III. Methods of Treating Constipation and/or a Constipation-Related Symptom with a “Fixed Dose” of Aminosterol

Aspects of this disclosure relate to methods of treating constipation and/or constipation-related symptoms, or disorders associated with constipation, by administration of a “fixed dose” of aminosterol as disclosed herein.

Example 1 provides a detailed protocol for determining a “fixed dose” based on improvement of one symptom associated with Parkinson's disease (PD), e.g., constipation. This example further details how this “fixed dose” successfully treated not only constipation, but also other non-dopamine related symptoms of PD.

As dopaminergic activity distinguishes PD from other neurodegenerative disorders and these data relate to symptoms that do not relate to this distinguishing feature, this dosing regime is believed to be extrapolatable both to constipation per se and constipation-related symptoms.

Not to be bound by theory, it is believed that establishing a patient-specific “fixed dose” based on obtaining a threshold improvement in any of the constipation-related symptoms described herein will successfully treat constipation and/or constipation-related symptoms. Further, to the extent that these symptoms are tied to an underlying disorder, administration of the therapeutically effective fixed dose is also believed to offer a means of treating, preventing, and/or delaying onset of an underlying disorder or disease causing the constipation or constipation-related symptoms.

Constipation refers to bowel movements that are infrequent or hard to pass. The stool is often hard and dry. Most commonly, constipation is thought of as infrequent bowel movements, usually less than 3 stools per week. Other symptoms may include abdominal pain, abdominal bloating, straining with bowel movements, excessive time needed to pass a bowel movement, hard stools, pain with bowel movements secondary to straining, and/or the sensation of incomplete bowel evacuation. Complications from constipation may include hemorrhoids, anal fissure or fecal impaction. The normal frequency of bowel movements in adults is between three per day and three per week. Babies often have three to four bowel movements per day while young children typically have two to three per day.

A variety of clinical scales can be used to evaluate constipation and related symptoms in the claimed methods. (Coffin & Caussé 2014). For example, the Rome III or IV Criteria are a set of symptoms that help standardize the diagnosis of constipation in various age groups. These criteria help physicians to better define constipation in a standardized manner. The Rome III Criteria for functional constipation must include two or more of the following and present for the past three months, with symptoms starting for at least 6 months prior to diagnosis: (1) straining during defecation for at least 25% of bowel movements; (2) lumpy or hard stools in at least 25% of defecations; (3) sensation of incomplete evacuation for at least 25% of defecations; (4) sensation of anorectal obstruction/blockage for at least 25% of defecations; (5) manual maneuvers to facilitate at least 25% of defecations; (6) fewer than 3 defecations per week; and (7) loose stools are rarely present without the use of laxatives. The Rome IV criteria for functional gastrointestinal disorders (FGIDs) published in 2016 has seen the addition of OIC to the section on Bowel Disorders, defined as ‘constipation triggered or worsened by opioid analgesics’.

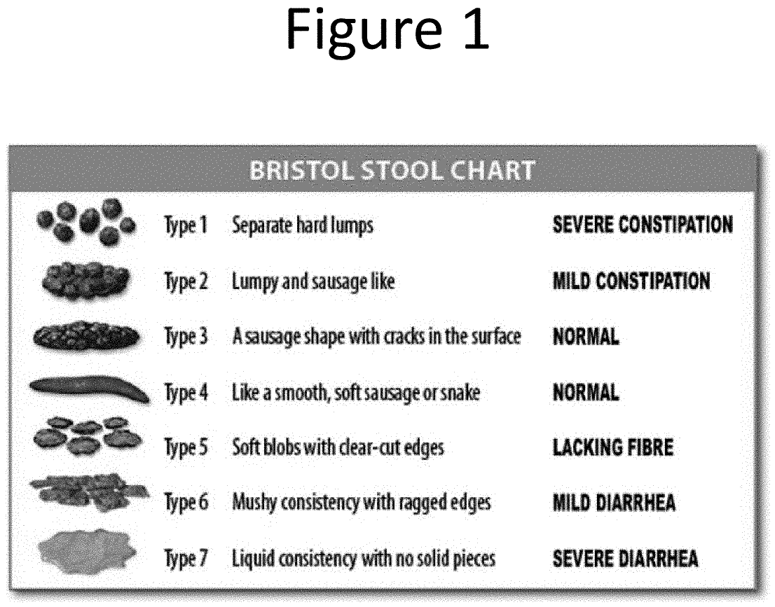

Another diagnostic medical tool used to evaluate constipation is the Bristol Stool Scale (also known as Bristol stool chart (BSC), Bristol stool form scale, or BSF scale) (FIG. 1), which is a medical aid designed to classify faeces into seven groups. The seven groups are: types 1 (separate hard lumps, like nuts (hard to pass); also known as goat faeces) and 2 (sausage-shaped, but lumpy) indicate constipation; types 3 (like a sausage but with cracks on its surface) and 4 (like a sausage or snake, smooth and soft) are ideal stools; and types 5 (soft blobs with clear cut edges (easy to pass), 6 (fluffy pieces with ragged edges, a mushy stool), and 7 (watery, no solid pieces, entirely liquid) may indicate diarrhea.

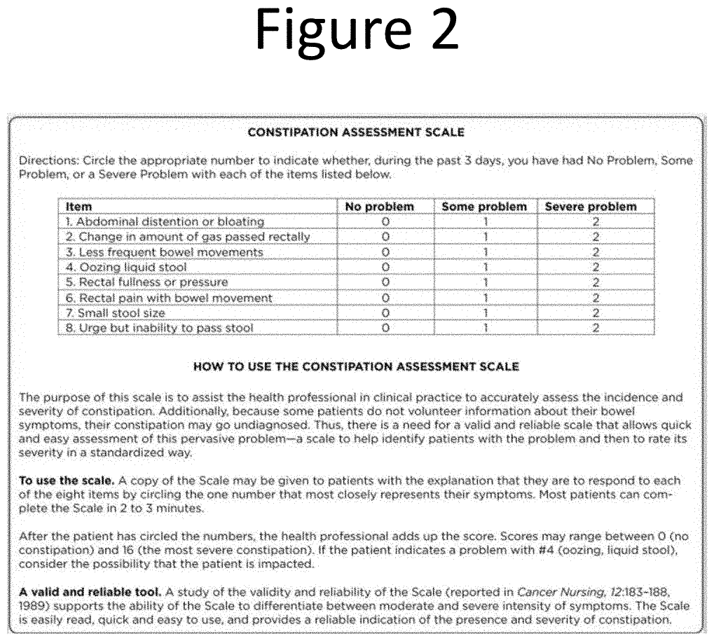

Yet another diagnostic tool used to assess constipation and related symptoms is the Constipation Assessment Scale (CAS), developed by McMillan and Williams (1989). The CAS (FIG. 2) was based on earlier research and clinical literature and includes eight commonly identified characteristics of constipation, including: (1) abdominal distension or bloating; (2) change in amount of gas passed rectally; (3) less frequent bowel movements; (4) oozing liquid stool; (5) rectal fullness or pressure; (6) rectal pain with bowel movement; (7) small stool size; and (8) urge but inability to pass stool (FIG. 2).

An additional diagnostic tool includes the Bowel Function Index (BFI), which is a patient-assessment scale developed and validated specifically for OIC. It is a physician-administered, easy-to-use scale made up of three items (ease of defecation, feeling of incomplete bowel evacuation, and personal judgement of constipation). An extensive analysis has been performed to validate the BFI as reliable, stable, clinically valid, and responsive to change in patients with OIC, with a 12-point change in score constituting a clinically relevant change in constipation.

Other known medically recognized tools for evaluating and measuring constipation include, for example, Unified Parkinson's Disease Scale (UPSRS), section 1.11 (Constipation Problems); the Constipation Scoring System (Agachan et al., 1996), Ease of Evacuation Scale (Andresen et al. 2007), and the Patient Assessment of Constipation (PAC) as well as two complementary components: the Symptom Questionnaire (PAC-SYM) and the Quality of Life Questionnaire (PAC-QOL). The PAC-SYM has twelve different categories, which are rated from 0 to 4, with 0=absent, 1=mild, 2=moderate, 3=severe, and 4=very severe. The twelve symptoms or categories rated are: discomfort in abdomen, pain in abdomen, bloating in abdomen, stomach cramps, painful bowel movements, rectal burning during or after bowel movement, rectal bleeding or tearing during or after a bowel movement, incomplete bowel movement, bowel movements that were too hard, bowel movements that were too small, straining or squeezing to try to pass bowel movements, and urge but inability to pass stool. The PAC-QOL asks five questions and has the subject rate the answers into five categories: 0=none of the time; 1=a little of the time; 2=some of the time; 3=most of the time; and 4=all of the time. The five questions are as follows: (1) have you had fewer bowel movements than you would like? (2) have you been satisfied with how often you have a bowel movement? (3) have you been satisfied with the regularity of your bowel movements? (4) have you been satisfied with the time it takes for food to pass through the intestines? (5) have you been satisfied with your treatment?

Constipation has many causes, which can be divided into congenital, primary, and secondary. Primary or functional constipation is defined by ongoing symptoms for greater than six months not due to an underlying cause such as medication side effects or an underlying medical condition. It is the most common kind of constipation, and is often multifactorial. Secondary causes include side effects of medications such as opiates, endocrine and metabolic disorders such as hypothyroidism, and obstruction such as from colorectal cancer. Celiac disease and non-celiac gluten sensitivity may also present with constipation.

Medications:

Many medications have constipation as a side effect. Some include (but are not limited to) opioids (“opioid-induced constipation”), diuretics, antidepressants, antihistamines, antispasmodics, anticonvulsants, tricyclic antidepressants, antiarrythmics, beta-adrenoceptor antagonists, anti-diarrheals, 5-HT3 receptor antagonists such as ondansetron, calcium channel blockers, antiacids, anticholinergics, and aluminum antacids. Certain calcium channel blockers such as nifedipine and verapamil can cause severe constipation due to dysfunction of motility in the rectosigmoid colon. Supplements such as calcium and iron supplements can also have constipation as a notable side effect. Of those taking opioids about 90% develop constipation.

Medical Conditions:

Metabolic and endocrine problems which may lead to constipation include, for example, hypercalcemia, hypothyroidism, hyperparathyroidism, porphyria, chronic kidney disease, pan-hypopituitarism, diabetes, diabetes mellitus, and cystic fibrosis. Constipation is also common in individuals with muscular and myotonic dystrophy.

Systemic diseases that may present with constipation include celiac disease, non-celiac gluten sensitivity, and systemic sclerosis. Constipation has a number of structural (mechanical, morphological, anatomical) causes, namely through creating space-occupying lesions within the colon that stop the passage of stool, such as colorectal/colon cancer, strictures, rectocoles, anal sphincter damage or malformation and post-surgical changes. Extra-intestinal masses such as other malignancies can also lead to constipation from external compression. Other diseases that can have constipation as a side effect include, but are not limited to, irritable bowel syndrome, diverticulitis, and inflammatory bowel disease.

Constipation also has neurological causes, including anismus, descending perineum syndrome, and Hirschsprung's disease. In infants, Hirschsprung's disease is the most common medical disorder associated with constipation. Anismus occurs in a small minority of persons with chronic constipation or obstructed defecation. Spinal cord lesions and neurological disorders such as Parkinson's disease and pelvic floor dysfunction or disorders can also lead to constipation.

Constipation is also associated with neurodegenerative diseases and conditions such as PD, Alzheimer's disease (AD), Huntington's chorea and/or Huntington's disease, Multiple Sclerosis, Amyotorphic Lateral Sclerosis (ALS), multiple system atrophy (MSA), schizophrenia, Friedreich's ataxia, vascular dementia, Lewy Body dementia or disease, spinal muscular atrophy, supranuclear palsy, fronto temperal dementia, progressive nuclear palsy, Guadeloupian Parkinsonism, spinocerebellar ataxia, autism, dementia of aging, neuropathy of diabetes, peripheral sensory neuropathy, cerebral palsy, epilepsy, diabetic neuropathy, traumatic head and/or spine injury, stroke, and depression.

Constipation & PD:

Constipation is much more common among patients with PD than in the general population. There are 1M people suffering from Parkinson's Disease in the US, of which roughly 60%, or 600,000 suffer from chronic constipation and in most, the condition is chronic, severe and unresponsive to standard therapy. This represents an economic burden to the individual with PD and to the healthcare system. According to the most recent Federal Supply Schedule (FSS; April 2016), the average 30-day reimbursed price for a basket of orally administered drugs for constipation is approximately $260 or $3120 per year. This represents about $1.8B of prescription laxatives just for patients with PD.

Constipation not only constitutes a major economic burden, but it also significantly affects the quality of life of the individual, contributing to social isolation and depression. Furthermore, the severity of the symptoms correlates negatively with patient reported quality of life.

While often dismissed as strictly a gastrointestinal symptom, constipation is believed to be an early indicator of neurodegenerative disease to the extent that ENS degeneration can be indicative of later CNS degeneration. Indeed, not to be bound by theory, but constipation is believed to be one of the earliest indicators of PD pathology. Accordingly, method embodiments disclosed herein relate to the treatment of constipation or the treatment and/or prevention of an underlying disorder associated with constipation.

Constipation is common in PD and often becomes symptomatic years before the onset of the motor dysfunction and the subsequent diagnosis of PD. There is substantial evidence that the neurodegenerative process associated with PD, namely the accumulation of toxic aggregates of alpha-synuclein, occurs within the enteric nervous system years before they appear within the brain. It is believed that the enteric nervous system (ENS), with its vast surface area, is subject to continuous insults from infectious agents and toxic substances. Although the function of alpha-synuclein is not known, inflammation within the nervous system leads to an increase in its intracellular levels. In individuals with PD the increase in alpha-synuclein leads to the formation of neurotoxic aggregates, perhaps because of a failure by the neuron (due to genetic factors) to effectively dispose of them. The aggregates of alpha-synuclein then traffic along the vagal nerve to the dorsal motor nucleus within the brainstem, and from there to more rostral structures.

The individual with PD suffers from a form of constipation that is believed to be caused principally by delayed transit through the colon. In addition, defecation is often impaired by dysfunction of the PD subject's anorectal reflex. For many individuals, bowel issues represent a significant detriment to quality of life. Failure to effectively manage this problem can also lead to bowel obstruction, especially as the terminal phase of PD approaches. A limited number of therapies have been subjected to clinical trials and they include agents that increase the fluid content of the stool, either by blocking fluid resorption or increasing the osmolar load within the intestine.

Constipation is a major clinical component of PD and is reported to occur in greater than 60% of affected individuals. The pathophysiological basis of constipation in PD is generally believed to be due to delayed transit through the colon. Several studies have demonstrated that transit of stool through the colon of an individual with PD is about 50% that measured in age matched controls. As a consequence, both stool frequency and stool consistency are abnormal in PD. For many patients, as well as those caring for these individuals, constipation remains a significant morbidity associated with the condition.

Few placebo-controlled clinical trials have been conducted in the PD population to assess the efficacy of therapeutics that could be of value. Addition of fiber to the diet, although increasing stool volume, is reported to have no effect on colon transit time. An osmotic laxative, polyethylene glycol (Magrogol) has been studied in a small placebo controlled clinical trial of individuals with mild constipation, and shown to provide benefit with respect to stool frequency and consistency. A short term placebo controlled trial of Lubiprostone, a chloride channel activator which increases intestinal fluid secretion, was only effective in about 50% of those treated, and resulted in passage of loose stools/diarrhea in place of constipation. Furthermore, Lubiprostone delays gastric emptying, a function already compromised in PD.

The pathophysiology of the gastrointestinal (GI) dysfunction in PD involves deposition of alpha-synuclein within both the ENS as well as within the brainstem. For reasons that remain unknown alpha-synuclein, which is a protein normally produced in neurons, forms neurotoxic intracellular aggregates in PD. Numerous studies suggest that the alpha-synuclein aggregate formation begins in the ENS of the PD individual many years before the onset of the motor symptoms. As a consequence of the normal retrograde neuronal trafficking that occurs within the vagus nerve, toxic aggregates are transported from the neurons of the ENS to the dorsal motor nucleus of the vagus, and then, gradually to sites within the brain that are involved in physical movement and balance. Because the constipation is fundamentally of an acquired neurodegenerative nature, it differs from other forms of this condition.

A. Methods of Treating Constipation

Example 1 demonstrates that administration of an aminosterol can displace αS from membranes in vitro and reduce the formation of neurotoxic αS aggregates in vivo, thereby stimulating gastrointestinal motility in patients with PD to relieve constipation. The observation that the dose required to achieve a prokinetic response increases with constipation severity supports the hypothesis that the greater the burden of αS impeding neuronal function, the higher the dose of aminosterol required to restore normalcy. The persistence of amelioration of CNS symptoms beyond the dosing period suggests the possibility of prolonged benefit.

Example 1 describes several tools used to measure and evaluate the effect of aminosterol treatment on constipation, including for example:

(1) Rome-IV Criteria for Constipation (7 criteria, with constipation diagnosis requiring two or more of the following: (i) straining during at least 25% of defecations, (ii) lumpy or hard stools in at least 25% of defecations, (iii) sensation of incomplete evacuation for at least 25% of defecations, (iv) sensation of anorectal obstruction/blockage for at least 25% of defecations; (v) manual maneuvers to facilitate at least 25% of defecations; (vi) fewer than 3 defecations per week; and (vii) loose stools are rarely present without the use of laxatives;

(2) Constipation—Ease of Evacuation Scale (from 1-7, with 7=incontinent, 4=normal, and 1=manual disimpaction);

(3) Bristol Stool Chart, which is a patient-friendly means of categorizing stool characteristics (assessment of stool consistency is a validated surrogate of intestinal motility) and stool diary;

(4) Unified Parkinson's Disease Scale (UPSRS), section 1.11 (Constipation Problems);

(5) Patient Assessment of Constipation Symptoms (PAC-SYM); and

(5) Patient Assessment of Constipation Quality of Life (PAC-QOL).

Examples of characteristics of constipation that can be positively affected by the method of the invention are described herein and include, but are not limited to, frequency of constipation, duration of constipation symptoms, bowel movement frequency, stool consistency, abdominal pain, abdominal bloating, incomplete evacuation or bowel movement, unsuccessful attempts at evacuation, painful defecation/rectal pain with bowel movement, straining during defecation and/or straining or squeezing to try to pass bowel movements, fecal incontinence/encopresis, abdominal discomfort, stomach cramps, stool consistency, rectal burning during or after bowel movement, rectal bleeding or tearing during or after a bowel movement, ease of defecation/passing stool, sensation of incomplete bowel evacuation, sensation of anorectal obstruction/blockage, bowel movements that were too hard, bowel movements that were too small, change in amount of gas passed rectally, less frequent bowel movements, oozing liquid stool, rectal fullness or pressure; small stool size; urge but inability to pass stool; and/or personal judgement of constipation.

Potentially all of these characteristics can be positively impacted by the methods of the invention. Further, assessments of these characteristics can be done using clinically recognized scales, as described herein.

The methods of using a therapeutically effective fixed dose of an aminosterol composition according to the invention to treat and/or prevent constipation preferably results in an improvement in one or more characteristic or symptoms of constipation, which can be measured using a clinically recognized scale. For example, one characteristic or symptom of constipation that can be evaluated is the number of CSBM or SMB per week, with an increase in the number of CSBM or SBM per week demonstrating a desired escalated aminosterol dose. The increase in CSBM or SBM can be, for example, an increase of between 1 to 3 spontaneous bowel movements in a week, or, optionally, full restoration of regular bowel function.

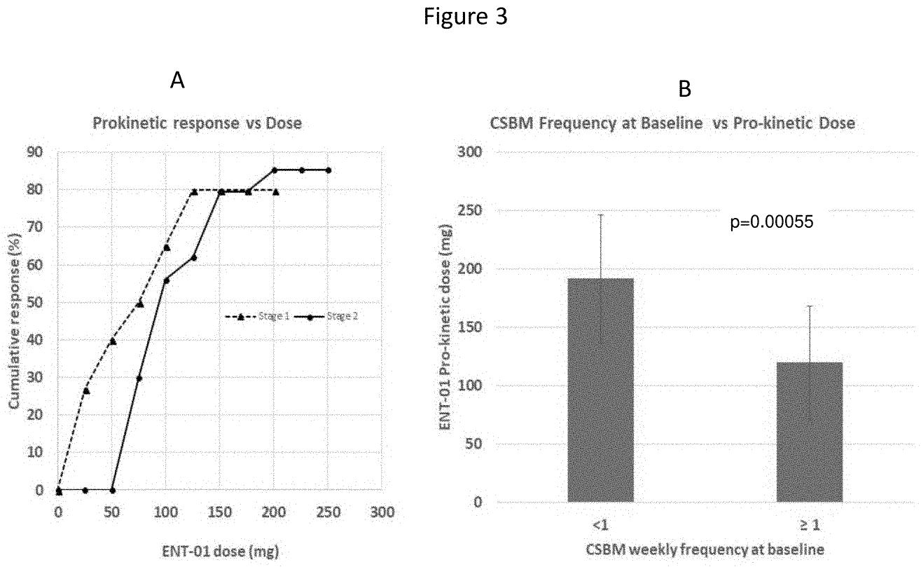

Data detailed in Example 1 shows that 80% of subjects responded to aminosterol treatment with improved bowel function (see FIG. 3A), with the cumulative response rate increasing in a dose-dependent fashion from 25% at 25 mg to a maximum of 80% at 200 mg (Stage 1, FIG. 3A). In Stage 2 of the study, the response rate increased in a dose-dependent fashion from 26% at 75 mg to 85.3% at 250 mg (FIG. 3A). The dose required for a bowel response was patient-specific and varied from 75 mg to 250 mg. The median efficacious dose was 100 mg.

The average CSBM/week increased from 1.2 at baseline to 3.8 at fixed dose (216% improvement) and SBM/week increased from 2.6 at baseline to 4.5 at fixed dose (73% improvement). Use of rescue medication decreased from 1.8/week at baseline to 0.3 at fixed dose (83% decrease). Consistency based on the Bristol stool scale also improved, increasing from mean 2.7 to 4.1 (52% improvement) and ease of passage increased from 3.2 to 3.7 (16% improvement). Subjective indices of wellbeing (PAC-QOL) and constipation symptoms (PAC-SYM) also improved during treatment.

The dose that proved efficacious in inducing a bowel response was strongly related to constipation severity at baseline (FIG. 3B); patients with baseline constipation of <1 CSBM/week required higher doses for a response (mean 192 mg) than patients with ≥1 CSBM/week (mean 120 mg).

In one embodiment of the invention, treatment of a subject having constipation and/or one or more constipation-related symptoms with an aminosterol in a method described herein results in an improvement of one or more characteristics of constipation. The improvement can be, for example, about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, about 300, about 325, about 350, about 375 or about 400%, optionally as measured using a clinically recognized scale or tool. Examples of constipation characteristics that can be measured and improved by the methods of the invention are described herein.

One surprising discovery that resulted from the experiments described herein relates to aminosterol dosing. It was surprisingly discovered that the dose of aminosterol required to obtain a positive impact on a symptom being evaluated, referred to herein as a “fixed escalated aminosterol dose,” is patient specific. Moreover, it was discovered that the fixed escalated aminosterol dose is not dependent upon age, size, or weight but rather is individually calibrated. Further, it was discovered that the severity of constipation correlates with a higher required “fixed escalated aminosterol dose.” It is theorized that the aminosterol dose required to obtain a positive effect in a subject for the symptom being evaluated correlates with the extent of neuronal damage. Thus, it is theorized that greater neuronal damage correlates with a higher required aminosterol dose to obtain a positive effect in a subject for the symptom being evaluated. The observation that the aminosterol dose required to achieve a desired response increases with constipation severity supports the hypothesis that the greater the burden of αS impeding neuronal function, the higher the dose of aminosterol required to restore normal bowel function. Moreover, the data described in Example 1 confirms the hypothesis that gastrointestinal dysmotility in PD results from the progressive accumulation of αS in the ENS, and that aminosterol treatment can restore neuronal function by displacing αS and stimulating enteric neurons. These results demonstrate that the ENS in PD is not irreversibly damaged and can be restored to normal function.

In calibrating the fixed aminosterol dose for a specific patient, the starting dose is varied based upon the severity of the constipation. Thus, for subjects with severe constipation, e.g., subjects with 1 or less CSBM or SMB per week, oral aminosterol dosing is started at about 100 to about 175 mg or more (or any amount in-between these values as described herein). For subjects with less severe constipation, e.g., more than 1 CSBM or SBM per week, oral aminosterol dosing is started at about 25 to about 75 mg (or any amount in-between these values as described herein). Dosing for both patients is then escalated by defined amounts over a defined period of time until the fixed escalated dose for the patient is identified.

B. Neurodiseases with Constipation-Related Symptom

The methods and aminosterol compositions of the invention can be used to treat constipation and/or constipation-related symptoms associated with neurodiseases such as Parkinson's Disease, Alzheimer's disease (AD), Huntington's chorea and/or Huntington's disease, Multiple Sclerosis, Amyotorphic Lateral Sclerosis (ALS), multiple system atrophy (MSA), schizophrenia, Friedreich's ataxia, vascular dementia, Lewy Body dementia or disease, spinal muscular atrophy, supranuclear palsy, fronto temperal dementia, progressive nuclear palsy, Guadeloupian Parkinsonism, spinocerebellar ataxia, autism, dementia of aging, neuropathy of diabetes, peripheral sensory neuropathy, cerebral palsy, epilepsy, diabetic neuropathy, traumatic head and/or spine injury, stroke, and depression.

Data described in Example 1 shows remarkable improvement in a wide variety of symptoms correlated with PD, including a significant and positive effect on bowel function and neurologic symptoms of PD. The study is the first proof of concept demonstration that directly targeting αS pharmacologically can achieve beneficial GI, autonomic and CNS responses in neurodiseases such as PD.

For example, regarding the effect on bowel function, in Stage 1 (single dose), cumulative response rate increased in a dose-dependent fashion from 25% at 25 mg to a maximum of 80% at 200 mg (FIG. 3A). In Stage 2 (daily dosing), the response rate increased in a dose-dependent fashion from 26% at 75 mg to 85.3% at 250 mg (FIG. 3A). The dose required for a bowel response was patient-specific and varied from 75 mg to 250 mg. Median efficacious dose was 100 mg. Average CSBM/week increased from 1.2 at baseline to 3.8 at fixed dose and SBM increased from 2.6 at baseline to 4.5 at fixed dose. Use of rescue medication decreased from 1.8/week at baseline to 0.3 at fixed dose. Consistency based on the Bristol stool scale also improved, increasing from mean 2.7 to 4.1 and ease of passage increased from 3.2 to 3.7. Subjective indices of wellbeing (PAC-QOL) and constipation symptoms (PAC-SYM) also improved during treatment. While the improvement in most stool-related indices did not persist beyond the treatment period, CSBM frequency remained significantly above baseline value.

### IV. Combination Therapy

In the methods of the invention, the aminosterol compositions may be administered alone or in combination with other therapeutic agents. An example of an additional therapeutic agent is one known to be useful in treating constipation and/or a constipation-related symptom. Conventional treatment of constipation can include, for example, increased fluid intake, fiber supplements, laxatives, physical intervention, and surgical intervention.

In addition, as noted above, the methods of the invention are useful in treating and/or preventing the conditions described herein, including but not limited to constipation, constipation-related symptoms, chronic idiopathic constipation, Opioid induced constipation, Irritable bowel syndrome, Inflammatory Bowel Disease, Diabetes mellitus, Parkinson's disease, Alzheimer's disease, dementia of aging, Huntington's chorea and/or Huntington's disease, neuropathy of diabetes, peripheral sensory neuropathy, cerebral palsy, epilepsy, diabetic neuropathy, traumatic head and/or spine injury, stroke, Amyotrophic lateral sclerosis (ALS), multiple sclerosis, multiple system atrophy (MSA), schizophrenia, Friedreich's ataxia, vascular dementia, Lewy Body dementia or disease, spinal muscular atrophy, supranuclear palsy, fronto temperal dementia, progressive nuclear palsy, Guadeloupian Parkinsonism, spinocerebellar ataxia, autism, depression, and certain malignancies.

Thus, any active agent known to be useful in treating these conditions can be used in the methods of the invention, and either combined with the aminosterol compositions used in the methods of the invention, or administered separately or sequentially.

For example, in methods of treating constipation and/or constipation-related symptoms associated with Irritable bowel syndrome, the aminosterol composition can be co-administered or combined with drugs commonly prescribed to treat IBS or related symptoms, such as alosetron hydrochloride (Lotronex®), fiber supplements or laxatives for constipation or medicines to decrease diarrhea, such as diphenoxylate and atropine (Lomotil®) or loperamide (Imodium®). An antispasmodic is commonly prescribed for treating IBS, which helps control colon muscle spasms and reduce abdominal pain. Antidepressants may relieve some symptoms of IBS. However, both antispasmodics and antidepressants can worsen constipation, so some doctors will also prescribe medications that relax muscles in the bladder and intestines, such as belladonna alkaloid combinations and phenobarbital (Donnatal®) and chlordiazepoxide and clidinium bromide (Librax®).

In methods of treating constipation and/or constipation-related symptoms associated with Inflammatory Bowel Disease, the aminosterol composition can be co-administered or combined with drugs commonly prescribed to treat Inflammatory Bowel Disease or related symptoms, such as aminosalicylates, corticosteroids, immune modifiers, anti-tumor necrosis factor (TNF) agents, and antibiotics. Exemplary aminosalicylates include but are not limited to sulfasalazine (Azulfidine®), mesalamine (Asacol®, Pentasa®), olsalazine (Dipentum®), and balsalazide (Colazal®). Exemplary corticosteroids include but are not limited to methylprednisolone, prednisone, prednisolone, budesonide, dexamethasone, hydrocortisone, betamethasone, cortisone, prednisolone, and triamcinolone. Exemplary immune modifiers include but are not limited to 6-mercaptopurine (6-MP, Purinethol®) and azathioprine (Imuran®). An exemplary anti-TNF agent includes but is not limited to infliximab (Remicade®). Exemplary antibiotics include but are not limited to metronidazole and ciprofloxacin. Additional examples of antibiotic agents include, but are not limited to, aminoglycosides, Ansamycins, Carbacephems, Carbapenems, Cephalosporins, Glycopeptides, Macrolides, Monobactams, Penicillins, Polypeptides, Polymyxin, Quinolones, Sulfonamides, Tetracyclines, and others (e.g., Arsphenamine, Chloramphenicol, Clindamycin, Lincomycin, Ethambutol, Fosfomycin, Fusidic acid, Furazolidone, Isoniazid, Linezolid, Metronidazole, Mupirocin, Nitrofurantoin, Platensimycin, Pyrazinamide, Quinupristin/Dalfopristin, Rifampicin (Rifampin in US), Thiamphenicol, Tinidazole, Dapsone, and lofazimine). Examples of these classes of antibiotics include, but are not limited to, Amikacin®, Gentamicin®, Kanamycin®, Neomycin®, Netilmicin®, Streptomycin®, Tobramycin®, Paromomycin®, Geldanamycin®, Herbimycin®, Loracarbef®, Ertapenem®, Doripenem®, Imipenem®/Cilastatin®, Meropenem®, Cefadroxil®, Cefazolin®, Cefalotin® or Cefalothin®, Cefalexin®, Cefaclor®, Cefamandole®, Cefoxitin®, Cefprozil®, Cefuroxime®, Cefixime®, Cefdinir®, Cefditoren®, Cefoperazone®, Cefotaxime®, Cefpodoxime®, Ceftazidime®, Ceftibuten®, Ceftizoxime®, Ceftriaxone®, Cefepime®, Ceftobiprole®, Teicoplanin®, Vancomycin®, Azithromycin®, Clarithromycin®, Dirithromycin®, Erythromycin®, Roxithromycin®, Troleandomycin®, Telithromycin®, Spectinomycin®, Aztreonam®, Amoxicillin®, Ampicillin®, Azlocillin®, Carbenicillin®, Cloxacillin®, Dicloxacillin®, Flucloxacillin®, Mezlocillin®, Meticillin®, Nafcillin®, Oxacillin®, Penicillin®, Piperacillin®, Ticarcillin®, Bacitracin®, Colistin®, Polymyxin® B, Ciprofloxacin®, Enoxacin®, Gatifloxacin®, Levofloxacin®, Lomefloxacin®, Moxifloxacin®, Norfloxacin®, Ofloxacin®, Trovafloxacin®, Grepafloxacin®, Sparfloxacin®, Temafloxacin®, Mafenide®, Sulfonamidochrysoidine® (archaic), Sulfacetamide®, Sulfadiazine®, Sulfamethizole®, Sulfanilimide® (archaic), Sulfasalazine®, Sulfisoxazole®, Trimethoprim®, rimethoprim-Sulfamethoxazole® (Co-trimoxazole) (TMP-SMX), Demeclocycline®, Doxycycline®, Minocycline®, Oxytetracycline®, and Tetracycline.

In methods of treating constipation and/or constipation-related symptoms associated with Diabetes and/or diabetes mellitus, including both Type 1 and Type 2 diabetes, or neuropathy of diabetes, the aminosterol composition can be co-administered or combined with drugs commonly prescribed to treat Diabetes mellitus or related symptoms, such as insulin (ular and NPH insulin, or synthetic insulin analogs) (e.g., Humulin®, Novolin®) and oral antihyperglycemic drugs. Oral antihyperglycemic drugs include but are not limited to (1) biguanides such as metformin (Glucophage®), (2) Sulfonylureas such as acetohexamide, chlorpropamide (Diabinese®), glimepiride (Amaryl®), Glipizide (Glucotrol®), Tolazamide, Tolbutamide, and glyburide (Diabeta®, Micronase®), (3) Meglitinides such as repaglinide (Prandin®) and nateglinide (Starlix®), (4) Thiazolidinediones such as rosiglitazone (Avandia®) and pioglitazone (Acto®), (5) Alpha-glucosidase inhibitors such as acarbose (Precose®) and miglitol (Glyset®), (6) Dipeptidyl peptidase-4 inhibitors such as Sitagliptin (Januvia®), (7) Glucagon-like peptide agonists such as exenatide (Byetta®), and (8) Amylin analogs such as pramlintide (Symlin®).

In methods of treating constipation and/or constipation-related symptoms associated with Parkinson's disease, the aminosterol composition can be co-administered or combined with drugs commonly prescribed to treat Parkinson's disease or related symptoms, such as levodopa (usually combined with a dopa decarboxylase inhibitor or COMT inhibitor), dopamine agonists and MAO-B inhibitors. Exemplary dopa decarboxylase inhibitors are carbidopa and benserazide. Exemplary COMT inhibitors are tolcapone and entacapone. Dopamine agonists include, for example, bromocriptine, pergolide, pramipexole, ropinirole, piribedil, cabergoline, apomorphine, lisuride, and rotigotine. MAO-B inhibitors include, for example, selegiline and rasagiline. Other drugs commonly used to treat Parkinson's disease include, for example, amantadine, anticholinergics, clozapine for psychosis, cholinesterase inhibitors for dementia, and modafinil for daytime sleepiness.

In methods of treating constipation and/or constipation-related symptoms associated with Alzheimer's disease, the aminosterol composition can be co-administered or combined with drugs commonly prescribed to treat Alzheimer's disease or related symptoms, such as Glutamate, Antipsychotic drugs, Huperzine A, acetylcholinesterase inhibitors and NMDA receptor antagonists such as memantine (Akatinol®, Axura®, Ebixa®/Abixa®, Memox® and) Namenda®. Examples of acetylcholinesterase inhibitors are donepezil (Aricept®), galantamine (Razadyne®), and rivastigmine (Exelon®).

In methods of treating constipation and/or constipation-related symptoms associated with Huntington's chorea or disease, the aminosterol composition can be co-administered or combined with drugs commonly prescribed to treat Huntington's chorea or related symptoms, such as medications prescribed to help control emotional and movement problems associated with Huntington's chorea. Such medications include, but are not limited to, (1) antipsychotic drugs, such as haloperidol and clonazepam, (2) drugs used to treat dystonia, such as acetylcholine-regulating drugs (trihexyphenidyl, benztropine (Cogentin®), and procyclidine HCl); GABA-regulating drugs (diazepam (Valium®), lorazepam (Ativan®), clonazepam (Klonopin®), and baclofen (Lioresal®)); dopamine-regulators (levodopa/carbidopa (Sinemet®), bromocriptine (parlodel)), reserpine, tetrabenazine; anticonvulsants (carbamazepine (Tegretol®); and Botulinum toxin (Botox®); and (3) drugs used to treat depression (fluoxetine, sertraline, and nortriptyline). Other drugs commonly used to treat HD include amantadine, tetrabenazine, Dopamine blockers, and co-enzyme Q10.

In methods of treating constipation and/or constipation-related symptoms associated with peripheral sensory neuropathy, the aminosterol composition can be co-administered or combined with drugs commonly prescribed to treat peripheral sensory neuropathy or related symptoms. Peripheral sensory neuropathy refers to damage to nerves of the peripheral nervous system, which may be caused either by diseases of or trauma to the nerve or the side-effects of systemic illness. Drugs commonly used to treat this condition include, but are not limited to, neurotrophin-3, tricyclic antidepressants (e.g., amitriptyline), antiepileptic therapies (e.g., gabapentin or sodium valproate), synthetic cannabinoids (Nabilone) and inhaled cannabis, opiate derivatives, and pregabalin (Lyrica®).

In methods of treating constipation and/or constipation-related symptoms associated with traumatic head and/or spine injury, the aminosterol composition can be co-administered or combined with drugs commonly prescribed to treat traumatic head and/or spine injury or related symptoms, such as analgesics (acetaminophen, NSAIDs, salicylates, and opioid drugs such as morphine and opium) and paralytics.

In methods of treating constipation and/or constipation-related symptoms associated with stroke, the aminosterol composition can be co-administered or combined with drugs commonly prescribed to treat stroke or related symptoms, such as aspirin, clopidogrel, dipyridamole, tissue plasminogen activator (tPA), and anticoagulants (e.g., alteplase, Warfarin, dabigatran).

In methods of treating constipation and/or constipation-related symptoms associated with Amyotrophic lateral sclerosis, the aminosterol composition can be co-administered or combined with drugs commonly prescribed to treat Amyotrophic lateral sclerosis or related symptoms, such as riluzole (Rilutek®), KNS-760704 (an enantiomer of pramipexole), olesoxime (TR019622), talampanel, Arimoclomol, medications to help reduce fatigue, ease muscle cramps, control spasticity, reduce excess saliva and phlegm, control pain, depression, sleep disturbances, dysphagia, and constipation.

In methods of treating constipation and/or constipation-related symptoms associated with multiple sclerosis, the aminosterol composition can be co-administered or combined with drugs commonly prescribed to treat multiple sclerosis or related symptoms, such as corticosteroids (e.g., methylprednisolone), plasmapheresis, fingolimod (Gilenya®), interferon beta-1a (Avonex®, CinnoVex®, ReciGen® and Rebif®), interferon beta-1b (Betaseron®, Betaferon®), glatiramer acetate (Copaxone®), mitoxantrone, natalizumab (Tysabri®), alemtuzumab (Campath®), daclizumab (Zenapax®), rituximab, dirucotide, BHT-3009, cladribine, dimethyl fumarate, estriol, fingolimod, laquinimod, minocycline, statins, temsirolimus teriflunomide, naltrexone, and vitamin D analogs.

In methods of treating constipation and/or constipation-related symptoms associated with cerebral palsy, the aminosterol composition can be co-administered or combined with drugs commonly prescribed to treat cerebral palsy or related symptoms, such as Botulinum toxin A injections.

In methods of treating constipation and/or constipation-related symptoms associated with epilepsy, the aminosterol composition can be co-administered or combined with drugs commonly prescribed to treat epilepsy or related symptoms, such as anticonvulsants (e.g., carbamazepine (Tegretol®), clorazepate (Tranxene®), clonazepam (Klonopin®), ethosuximide (Zarontin®), felbamate (Felbatol®), fosphenytoin (Cerebyx®), gabapentin (Neurontin®), lacosamide (Vimpat®), lamotrigine (Lamictal®), levetiracetam (Keppra®), oxcarbazepine (Trileptal®), phenobarbital (Luminal®), phenytoin (Dilantin®), pregabalin (Lyrica®), primidone (Mysoline®), tiagabine (Gabitril®), topiramate (Topamax®), valproate semisodium (Depakote®), valproic acid (Depakene®), and zonisamide (Zonegran®), clobazam (Frisium®), vigabatrin (Sabrir), retigabine, brivaracetam, seletracetam, diazepam (Valium®, Diastat®), lorazepam (Ativan®), paraldehyde (Paral®), midazolam (Versed®), pentobarbital (Nembutal®), acetazolamide (Diamox®), progesterone, adrenocorticotropic hormone (ACTH, Acthar®), various corticotropic steroid hormones (prednisone), and bromide.

In methods of treating constipation and/or constipation-related symptoms associated with depression, the aminosterol composition can be co-administered or combined with drugs commonly prescribed to treat depression, such as any of the class of Tricyclic antidepressants, Monoamine oxidase inhibitors, Selective serotonin reuptake inhibitors, and Serotonin and norepinephrine reuptake inhibitors.

In the methods of treating constipation and/or constipation-related symptoms associated with malignancies, the aminosterol composition can be co-administered or combined with drugs commonly used to treat malignancies. These include all known cancer drugs, such as but not limited to those listed at http://www.cancer.gov/cancertopics/druginfo/alphalist as of May 5, 2014, which is specifically incorporated by reference.

Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second. The regimen selected can be administered concurrently since activation of the aminosterol induced response does not require the systemic absorption of the aminosterol into the bloodstream and thus eliminate concern over the likelihood systemic of drug-drug interactions between the aminosterol and the administered drug.

### V. Definitions

The following definitions are provided to facilitate understanding of certain terms used throughout this specification.

Technical and scientific terms used herein have the meanings commonly understood by one of ordinary skill in the art, unless otherwise defined. Any suitable materials and/or methodologies known to those of ordinary skill in the art can be utilized in carrying out the methods described herein.

As used in the description of the invention and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein the term “aminosterol” encompasses squalamine or a derivative thereof, an isomer or prodrug of squalamine, Aminosterol 1436 or a derivative thereof, an isomer or prodrug of Aminosterol 1436, or a naturally occurring aminosterol isolated from Squalus acanthias or a derivative thereof, as described herein. “Amino sterols” useful in the invention also encompass a pharmaceutically equivalent salt of any aminosterol compound described herein. These compounds, and pharmaceutically acceptable salts thereof, are collectively referred to herein as “squalamine” and “aminosterols.” Thus, the term “aminosterol” as used herein is intended to encompass the broader class that includes both squalamine and the known naturally occurring aminosterols.

As used herein, “therapeutic activity” or “activity” may refer to an activity whose effect is consistent with a desirable therapeutic outcome in humans, or to desired effects in non-human mammals or in other species or organisms. Therapeutic activity may be measured in vivo or in vitro. For example, a desirable effect may be assayed in cell culture.

As used herein, the phrase “therapeutically effective amount” shall mean the drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

The term “administering” as used herein includes prescribing for administration as well as actually administering, and includes physically administering by the subject being treated or by another.

As used herein “subject,” “patient,” or “individual” refers to any subject, patient, or individual, and the terms are used interchangeably herein. In this regard, the terms “subject,” “patient,” and “individual” includes mammals, and, in particular humans. When used in conjunction with “in need thereof,” the term “subject,” “patient,” or “individual” intends any subject, patient, or individual having or at risk for a specified symptom or disorder.

The terms “treatment,” “treating,” or any variation thereof includes reducing, ameliorating, or eliminating (i) one or more specified symptoms and/or (ii) one or more symptoms or effects of a specified disorder. The terms “prevention,” “preventing,” or any variation thereof includes reducing, ameliorating, or eliminating the risk of developing (i) one or more specified symptoms and/or (ii) one or more symptoms or effects of a specified disorder.

The following examples are provided to illustrate the present invention. It should be understood, however, that the invention is not to be limited to the specific conditions or details described in these examples. Throughout the specification, any and all references to a publicly available document, including a U.S. patent, are specifically incorporated by reference.

### EXAMPLES

### Example 1

This example describes an exemplary method of treating and/or preventing constipation and/or constipation-related symptoms associated with Parkinson's disease (PD) in a clinical trial setting.

Overview:

The subjects of the trial all had PD and experienced constipation, which is a characteristic of PD. The primary objectives of the trial involving patients with PD and constipation were to evaluate the safety and pharmacokinetics of oral squalamine (ENT-01) and to identify the dose required to improve bowel function, which was used as a clinical endpoint.

Several non-constipation PD symptoms were also assessed as endpoints, including, for example, (1) sleep problems, including daytime sleepiness; (2) non-motor symptoms, such as (i) depression (including apathy, anxious mood, as well as depression), (ii) cognitive impairment (e.g., using trail making test and the UPDRS), (iii) hallucinations (e.g., using The University of Miami Parkinson's Disease Hallucinations Questionnaire (UM-PDHQ) and the UPDRS, (iv) dopamine dysregulation syndrome (UPDRS), (v) pain and other sensations, (vi) urinary problems, (vii) light headedness on standing, and (viii) fatigue (e.g., using Parkinson's Disease Fatigue Scale 9PFS-1t and the UPDRS); (3) motor aspects of experiences of daily living, such as (i) speech, (ii) saliva and drooling, (iii) chewing and swallowing, (iv) eating tasks, (v) dressing, (vi) hygiene, (vii) handwriting; (viii) doing hobbies and other activities, (ix) turning in bed, (x) tremor, (xi) getting out of bed, a car, or a deep chair, (xii) walking and balance, (xiii) freezing; (4) motor examination, such as (i) speech, (ii) facial expression, (iii) rigidity, (ix) finger tapping, (v) hand movements, (vi) pronation-supination movements of hands, (vii) toe tapping, (viii) leg agility, arising from chair, (ix) gait, (x) freezing of gait, (xi) postural stability, (xii) posture, (xiii) global spontaneity of movement (body bradykinesia), (xiv) postural tremor of the hands, (xv) kinetic tremor of the hands, (xvi) rest tremor amplitude, (xvii) constancy of rest tremor; (5) motor complications, such as (i) time spent with dyskinesias, (ii) functional impact of dyskinesias, (iii) time spent in the off state, (iv) functional impact of fluctuations, (v) complexity of motor fluctuations, and (vi) painful off-state dystonia.

Active Agent & Dosing:

Squlamaine (ENT-01; Enterin, Inc.) was formulated for oral administration in the trial. The active ion of ENT-01, squalamine, an aminosterol originally isolated from the dogfish shark, has been shown to reverse gastrointestinal dysmotility in several mouse models of PD. In addition, squalamine (ENT-01) has been shown to inhibit the formation of aggregates of αS both in vitro, and in a C. elegans model of PD in vivo (Perni et al. 2017). In the C. elegans model, squalamine produced a complete reversal of muscle paralysis.

ENT-01 is the phosphate salt of squalamine. For this study it was formulated as a small 25 mg coated tablet. Dosing ranged from 25 mg to 250 mg, with dosages greater than 25 mg requiring multiple pills (e.g., 50 mg=two 25 mg pills). Dosing instructions=take 60 mins before breakfast with 8 oz water. The dose was taken by each patient upon awakening on an empty stomach along with 8 oz. of water simultaneously to dopamine. The subject was not allowed to ingest any food for at least 60 minutes after study medication. Squalamine is highly charged and will adsorb to foodstuffs, so it was administered prior to feeding.

The phosphate salt of squalamine (ENT-01) is weakly soluble in water at neutral pH but readily dissolves at pH<3.5 (the pH of gastric fluid). Squalamine, as the highly water soluble dilactate salt has been extensively studied in over three Phase 1 and eight Phase 2 human clinical trials as an intravenous agent for the treatment of cancer and diabetic retinopathy. The compound is well tolerated in single and repeat intravenous administration, alone or in combination with other agents, to doses of at least 300 mg/m².

In the current clinical trial, squalamine (ENT-01) was administered orally to subjects with PD who had long term constipation. Although this trial was the first in man oral dosing study of ENT-01, humans have long been exposed to low doses of squalamine (milligram to microgram) in the various commercial dogfish shark liver extracts available as nutraceuticals (e.g., Squalamax). In addition, following systemic administration squalamine is cleared by the liver and excreted as the intact molecule (in mice) into the duodenum through the biliary tract. Drug related GI toxicology has not been reported in published clinical trials involving systemic administration of squalamine.

The starting dose in the Stage 1 segment of the trial was 25 mg (0.33 mg/kg for a 75 kg subject). The maximum single dose in Stage 1 was 200 mg (2.7 mg/kg for a 75 kg subject). The maximum dose evaluated in Stage 2 of the trial was 250 mg/day (3.3 mg/kg/day for a 75 kg subject), and the total daily dosing exposure lasted no longer than 25 days.

The daily dosing range in the clinical trial was from 25 mg/day (14.7 mg/m²) to 250 mg/day (147 mg/m²). Oral dosing of squalamine (ENT-01), because of its low oral bioavailability, is not anticipated to reach significant plasma concentrations in human subjects. In preclinical studies, squalamine (ENT-01) exhibited an oral bioavailability of about 0.1% in both rats and dogs. In Stage 1 of this phase 2 study, oral dosing up to 200 mg (114 mg/m²) yielded an approximate oral bioavailability of about 0.1%, based on a comparison of a pharmacokinetic data of the oral dosing and the pharmacokinetic data measured during prior phase 1 studies of IV administration of squalamine.

Study Protocol:

The multicenter Phase 2 trial was conducted in two Stages: a dose-escalation toxicity study in Stage 1 and a dose range-seeking and proof of efficacy study in Stage 2.

PD symptoms were assessed using a number of different tools:

(1) Numeric Rating Scales for Pain and Swelling (scale of 0-10, with 0=no pain and 10=worst pain ever experienced);

(2) Rome-IV Criteria for Constipation (7 criteria, with constipation diagnosis requiring two or more of the following: (i) straining during at least 25% of defecations, (ii) lumpy or hard stools in at least 25% of defecations, (iii) sensation of incomplete evacuation for at least 25% of defecations, (iv) sensation of anorectal obstruction/blockage for at least 25% of defecations; (v) manual maneuvers to facilitate at least 25% of defecations; (vi) fewer than 3 defecations per week; and (vii) loose stools are rarely present without the use of laxatives;

(3) Constipation—Ease of Evacuation Scale (from 1-7, with 7=incontinent, 4=normal, and 1=manual disimpaction);

(4) Bristol Stool Chart, which is a patient-friendly means of categorizing stool characteristics (assessment of stool consistency is a validated surrogate of intestinal motility) and Stool Diary;

(5) Sleep Diary (participants completed a sleep diary on a daily basis throughout the study. The diaries included time into bed and estimated time to sleep as well as wake time and duration during the night.);

(6) I-Button Temperature Assessment. The I-Button is a small, rugged self-sufficient system that measures temperature and records the results in a protected memory section. The Thermochron I-Button DS1921H (Maxim Integrated, Dallas, Tex.) was used for skin temperature measurement. I-Buttons were programmed to sample every 10 mins., and attached to a double-sided cotton sport wrist band using Velcro, with the sensor face of the I-Button placed over the inside of the wrist, on the radial artery of the dominant hand. Subjects removed and replaced the data logger when necessary (i.e., to have a bath or shower). The value of skin temperature assessment in sleep research is that the endogenous skin warming resulting from increased skin blood flow is functionally linked to sleep propensity. From the collected data, the mesor, amplitude, acrophase (time of peak temperature), Rayleight test (an index of interdaily stability), mean waveforms are calculated.);

(7) Non-motor Symptoms Questionnaire (NMSQ);

(8) Beck Depression Inventory (BDI-II);

(9) Unified Parkinson's Disease Rating Scale (UPDRS), which consists of 42 items in four subscales (Part I=Non-Motor Aspects of Experiences of Daily Living (nM-EDL) (1.1 cognitive impairment, 1.2 hallucinations and psychosis, 1.3 depressed mood; Part II=Motor Aspects of Experiences of Daily Living (M-EDL), Part III=Motor Examination, and Part IV=Motor Complications;

(10) Mini Mental State Examination (MMSE);

(11) Trail Making Test (TMT) Parts A and B;

(12) The University of Miami Parkinson's Disease Hallucinations Questionnaire (UM-PDHQ);

(13) Parkinson's Disease Fatigue Scale (PFS-16);

(14) Patient Assessment of Constipation Symptoms (PAC-SYM);

(15) Patient Assessment of Constipation Quality of Life (PAC-QOL);

(16) REM Sleep Behavior Disorder Screening Questionnaire; and

(17) Parkinson's Disease Sleep Scale.

Exploratory end-points, in addition to constipation, included for example, (i) depression assessed using the Beck Depression Inventory (BDI-II) (Steer et al. 2000) and Unified Parkinson's Disease Rating Scale (UPDRS); (ii) cognition assessed using the Mini Mental State Examination (MMSE) (Palsteia et al. 2018), Unified Parkinson's Disease Rating Scale (UPDRS), and Trail Making Test (TMT); (iii) sleep and REM-behavior disorder (RBD) using a daily sleep diary, I-Button Temperature Assessment, a REM sleep behavior disorder (RBD) questionnaire (RBDQ) (Stiasny-Kolster et al. 2007), and the UPDRS; (iv) hallucinations assessed using the PD hallucinations questionnaire (PDHQ) (Papapetropoulos et al. 2008), the UPDRS, and direct questioning; (v) fatigue using the Parkinson's Disease Fatigue Scale (PFS-16) and the UPDRS; (vi) motor functions using the UPDRS; and (vii) non-motor functions using the UPDRS.

Assessments were made at baseline and at the end of the fixed dose and washout periods. Circadian system status was evaluated by continuously monitoring wrist skin temperature (Thermochron iButton DS1921H; Maxim, Dallas) following published procedures (Sarabia et al. 2008).

Based on these data, it is believed that administration of squalamine (ENT-01), a compound that can displace αS from membranes in vitro, reduces the formation of neurotoxic αS aggregates in vivo, and stimulates gastrointestinal motility in patients with constipation. The observation that the dose required to achieve a prokinetic response increases with constipation severity supports the hypothesis that the greater the burden of αS impeding neuronal function, the higher the dose of squalamine (ENT-01) required to restore normal bowel function.

Study Design:

A multicenter Phase 2 trial was conducted in two Stages: a dose-escalation toxicity study in Stage 1 and a dose range-seeking and proof of efficacy study in Stage 2. The protocol was reviewed and approved by the institutional review board for each participating center and patients provided written informed consent.

Following successful screening, all subjects underwent a 14-day run-in period where the degree of constipation was assessed through a validated daily log (Zinsmeister et al. 2013) establishing baseline CSBMs/week. Subjects with an average of <3 CSBMs/week proceeded to dosing.

In Stage 1, ten (10) PD patients received a single escalating dose of squalamine (ENT-01) every 3-7 days beginning at 25 mg and continuing up to 200 mg or the limit of tolerability, followed by 2-weeks of wash-out. Duration of this part of the trial was 22-57 days. The 10 subjects in the sentinel group were assigned to Cohort 1 and participated in 8 single dosing periods. Tolerability limits included diarrhea or vomiting. A given dose was considered efficacious in stimulating bowel function (prokinetic) if the patient had a complete spontaneous bowel movement (CSBM) within 24 hours of dosing.

Each dose period was staggered, so that subjects 1-2 were administered a single dose of the drug at the lowest dose of 25 mg. Once 24 hours elapsed, and provided there were no safety concerns, the patient was sent home and brought back on day 4-8 for the next dose. During the days the subjects were home, they completed daily diaries and e-mailed them to the study coordinators. Subjects 3-10 were dosed after the first 2 subjects were observed for 72 hours, i.e. on Day 4. Subjects 1-2 were also brought back on Day 4-8 and given a single dose of 50 mg. Once another 24 hours elapsed and provided there were no safety concerns, the patients were all sent home and instructed to return on Day 7 for the next dosing level. This single dosing regimen was continued until each subject was given a single dose of 200 mg or reached a dose limiting toxicity (DLT). DLT was the dose which induced repeated vomiting, diarrhea, abdominal pain or symptomatic postural hypotension within 24 hours of dosing.

In Stage 2, 34 patients were evaluated. First, 15 new PD patients were administered squalamine (ENT-01) daily, beginning at 75 mg, escalating every 3 days by 25 mg to a dose that had a clear prokinetic effect (CSBM within 24 hours of dosing on at least 2 of 3 days at a given dose), or the maximum dose of 175 mg or the tolerability limit. This dose was then maintained (“fixed dose”) for an additional 3-5 days. After the “fixed dose”, these patients were randomly assigned to either continued treatment at that dose or to a matching placebo, for an additional 4-6 days prior to a 2-week wash-out.

A second cohort of 19 patients received squalamine (ENT-01) escalating from 100 mg/day to a maximum of 250 mg/day without subsequent randomization to squalamine (ENT-01) or placebo. Criteria for dose selection and efficacy were identical to those used in the previous cohort.

Patient Population:

Patients were between 18 and 86 years of age and diagnosed with PD by a clinician trained in movement disorders following the UK Parkinson's Disease Society Brain Bank criteria (Fahn et al. 1987). Patients were required to have a history of constipation as defined by <3 CSBMs/week and satisfy the Rome IV criteria for functional constipation (Mearin et al. 2016) at screening, which requires 2 or more of the following: (i) straining during at least 25% of defecations; (ii) lumpy or hard stools in at least 25% of defecations; (iii) sensation of incomplete evacuation in at least 25% of defecations; (iv) sensation of anorectal obstruction/blockage in at least 25% of defecations; and/or (v) manual maneuvers to facilitate at least 25% of defecations.

Baseline characteristics of patients are shown in Table 1. Patients in Stage 2 had somewhat longer duration of PD and higher UPDRS scores than participants in Stage 1.

TABLE 1
Baseline Characteristics of Dosed Patients

| | | | |
|---|---|---|---|
| | Stage 1** | Stage 2*** | Total |
| Characteristic | (n = 10) | (n = 34) | (n = 44) |

Sex- no. (%)

| | | | | | |
|---|---|---|---|---|---|
| Male | 5 (50) | 25 | (73.5) | 30 | (68.1) |
| Female | 5 (50) | 9 | (26.5) | 14 | (31.8) |
| White race-no. (%) | 8 (80) | 34 | (100) | 42 | (95.54) |

Age-yr

| | | | |
|---|---|---|---|
| Mean | 65.0 | 74.5 | 72.5 |
| Range | 58-70.5 | 60.6-84.2 | 58-84.2 |

Age at PD diagnosis-yr

| | | | |
|---|---|---|---|
| Mean | 61.1 | 67.7 | 66.2 |
| Range | 54.2-69 | 50.6-82.5 | 50.6-82.5 |

Duration of PD-yr

| | | | |
|---|---|---|---|
| Mean | 4.2 | 6.8 | 6.2 |
| Range | 1-11 | 0.3-17.3 | 0.3-17.3 |

Duration of constipation-yr

| | | | |
|---|---|---|---|
| Mean | 25.8 | 16.8 | 18.9 |
| Range | 1-65 | 0.5-66.0 | 0.5-66.0 |

UPDRS score

| | | | |
|---|---|---|---|
| Mean | 53.4 | 63.2 | 61.3 |
| Range | 33-88 | 24-122 | 24.0-122.0 |

Hoehn and Yahr-Stage

| | | | |
|---|---|---|---|
| Mean | 2.0 | 2.4 | 2.3 |
| Range | 2.0 | 1.0-5.0 | 1.0-5.0 |

Constipation severity* - CSBM/wk- no. (%)

| | | | | | |
|---|---|---|---|---|---|
| 0-1 | 8 (80) | 14 | (41.2) | 22 | (50) |
| 1.1-2 | 2 (20) | 17 | (50) | 19 | (43.2) |
| 2.1-3 | 0 | 3 | (8.8) | 3 | (6.8) |

*At baseline. Baseline value is the average number of CSBMs per week calculated at the end of the 2-week run-in period.
**In Stage 1, 10 patients received single escalating doses every 3-7 days starting at 25 mg and escalating up to dose limiting toxicity (DLT) or 200 mg, whichever came first, followed by a 2-week wash-out period.
***In Stage 2, 15 patients received daily doses starting at 75 mg and escalating every 3 days up to prokinetic dose (dose producing CSBMs on at least 2 of 3 days) or 175 mg, whichever came first, followed by an additional 2-4 days at that dose (“fixed dose” period) and were then randomized to treatment at the “fixed-dose” or placebo for 4-6 days. Wash-out lasted 2 weeks. The remaining 19 patients were escalated from 100 mg to prokinetic dose or 250 mg, whichever came first, followed by an additional 2-4 days at that dose and then a 2-week wash-out period.

Safety and Adverse Event (AE) Profile:

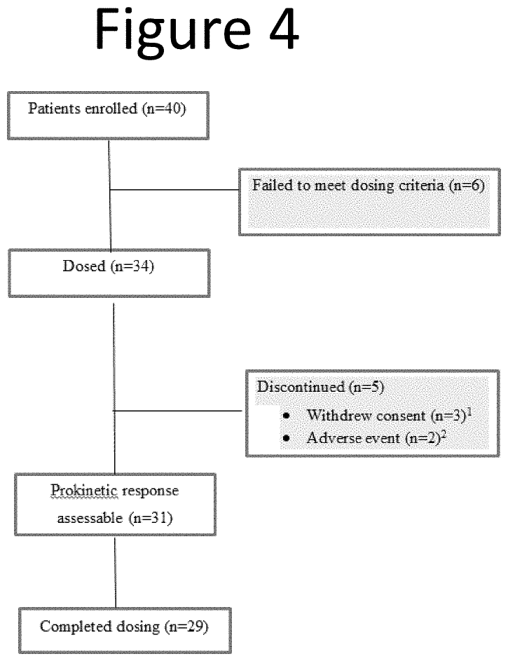

50 patients were enrolled and 44 were dosed. In Stage 1, 10 patients were dosed, 1 (10%) withdrew prior to completion and 9 (90%) completed dosing. In stage 2, 6 (15%) patients had ≥3 CSBM/week at the end of the run-in period and were excluded, 34 patients were dosed and bowel response was assessable in 31 (91%). Two patients (5.8%) were terminated prior to completion because of recurrent dizziness, and 3 others withdrew during dosing (8.8%): 2 because of diarrhea and 1 because of holiday. Fifteen patients were randomized. Study-drug assignments and patient disposition are shown in Table 2 and FIG. 4.

TABLE 2
Study drug assignments and adherence to treatment

| | |
|---|---|
| Stage 1 | Stage 2 |

| Enrolled | 10 | | 40 | |
|---|---|---|---|---|
| Failed prior to dosing | 0 | | 6 | |
| Dosed | 10 | | 34 | |
| 25-200 mg | 10 | | | |
| 75-175 mg | | | 19 | |
| 100-250 mg | | | 15 | |
| Terminated (%) | 0 | (0) | 2* | (5.8) |
| Withdrew (%) | 1 | (10) | 3 | (8.8) |
| Completed dosing (%) | 9 | (90) | 31** | (91) |

| | |
|---|---|
| Randomized | 15 |
| Treatment | 6 |
| Placebo | 9 |

The 2 patients who were terminated **29 patients completed dosing but an additional 2 who withdrew had an assessable prokinetic end-point.

Most AEs were confined to the GI tract (88% in Stage 1 and 63% in Stage 2). The most common AE was nausea which occurred in 4/10 (40%) patients in Stage 1 and in 18/34 (52.9%) in Stage 2 (Table 3). Diarrhea occurred in 4/10 (40%) patients in Stage 1 and 15/34 (44%) in Stage 2. One patient withdrew because of recurrent diarrhea. Other GI related AEs included abdominal pain 11/44 (32%), flatulence 3/44 (6.8%), vomiting 3/44 (6.8%), worsening of acid reflux 2/44 (4.5%), and worsening of hemorrhoids 1/44 (2.2%). One patient had a lower GI bleed (Serious adverse event, SAE) during the withdrawal period. This patient was receiving aspirin, naproxen and clopidogrel at the time of the bleed, and colonoscopy revealed large areas of diverticulosis and polyps. This SAE was considered unrelated to study medication. The only other noteworthy AE was dizziness 8/44 (18%). Dizziness was graded as moderate in one patient who was receiving an alpha-adrenergic blocking agent (Terazosin). This patient was withdrawn from the study and recovered spontaneously. All other AEs resolved spontaneously without discontinuation of squalamine (ENT-01). The relationship between dose and AEs is shown in Table 4.

TABLE 3
All adverse events (n, %)

Enrolled

| | |
|---|---|
| Stage 1 (n = 10) | Stage 2 (n = 40) |

Dosed

| | |
|---|---|
| 10 | 34 |

GI:
Nausea

| | | | |
|---|---|---|---|
| Mild | 4 (40) | 18 | (52) |
| Moderate | 0 | 1 | (2.9) |

Diarrhea

| | | | |
|---|---|---|---|
| Mild | 1 (10) | 12 | (35) |
| Moderate | 3 (30) | 2 | (5.8) |
| Severe | 0 | 1 | (2.9) |

Vomiting

| | | | |
|---|---|---|---|
| Mild | 1 (10) | 2 | (5.8) |

| | | |
|---|---|---|
| Moderate | 0 | 0 |

Abdominal pain

| | | | |
|---|---|---|---|
| Mild | 2 (20) | 4 | (11.7) |
| Moderate | 3 (30) | 2 | (5.8) |

Flatulence

| | | | |
|---|---|---|---|
| Mild | 2 (20) | 1 | (3) |

| | | |
|---|---|---|
| Moderate | 0 | 0 |

Loss of appetite*

| | | |
|---|---|---|
| Mild | 1 (10) | 0 |
| Moderate | 0 | 0 |

Worsening acid reflux

| | | | |
|---|---|---|---|
| Mild | 0 | 4 | (11.7) |

| | | |
|---|---|---|
| Moderate | 0 | 0 |

Worsening hemorrhoid

| | | | |
|---|---|---|---|
| Mild | 0 | 1 | (3) |

| | | |
|---|---|---|
| Moderate | 0 | 0 |

Lower GI bleed**

| | | | |
|---|---|---|---|
| Severe | 0 | 1 | (2.5) |

Non-GI:
Dizziness

| | | | |
|---|---|---|---|
| Mild | 0 | 7 | (20.5) |
| Moderate | 0 | 1 | (2.9) |

Blood in urine*

| | | |
|---|---|---|
| Mild | 1 (10) | 0 |
| Moderate | 0 | 0 |

Headache

| | | | |
|---|---|---|---|
| Mild | 1 (10) | 3 | (8.8) |

| | | |
|---|---|---|
| Moderate | 0 | 0 |

Urinary retention

| | | | |
|---|---|---|---|
| Mild | 0 | 1 | (3) |

| | | |
|---|---|---|
| Moderate | 0 | 0 |

Urinary tract infection

| | | | |
|---|---|---|---|
| Mild | 0 | 1 | (3) |
| Moderate | 0 | 2 | (5.8) |

Increased urinary frequency

| | | | |
|---|---|---|---|
| Mild | 0 | 2 | (5.8) |

| | | |
|---|---|---|
| Moderate | 0 | 0 |

Skin lesions-rash

| | | | |
|---|---|---|---|
| Mild | 0 | 3 | (8.8) |

| | | |
|---|---|---|
| Moderate | 0 | 0 |

Eye infection

| | | | |
|---|---|---|---|
| Mild | 0 | 1 | (3) |

| | | |
|---|---|---|
| Moderate | 0 | 0 |

Difficulty falling asleep

| | | | |
|---|---|---|---|
| Mild | 0 | 1 | (3) |

| | | |
|---|---|---|
| Moderate | 0 | 0 |

*Unrelated to ENT-01
**colonic diverticulosis, polyp, patient on aspirin, Plavix and naproxen. Unrelated to ENT-01

TABLE 4
Common adverse events by dose

| Dose | Stage 1 | | | Stage 2 | | |
|---|---|---|---|---|---|---|
| (mg) | Diarrhea | Nausea | Vomiting | Diarrhea | Nausea | Dizziness* |
| 0 | 0 | 0 | 0 | 1 | 0 | 2 |
| 25 | 1 | 0 | 0 | — | — | — |
| 50 | 1 | 0 | 0 | — | — | — |
| 75 | 1 | 0 | 0 | 7 | 3 | 8 |
| 100 | 0 | 1 | 1 | 10 | 12 | 7 |
| 125 | 1 | 2 | 1 | 3 | 4 | 8 |
| 150 | 1 | 0 | 0 | 2 | 11 | 2 |
| 175 | 1 | 1 | 0 | 1 | 12 | 0 |
| 200 | 0 | 2 | 0 | 3 | 6 | — |
| 225 | — | — | — | 3 | 1 | |
| 250 | — | — | — | 2 | — | |

*lightheadedness included

TABLE 5
Dose limiting toxicity criteria

| | |
|---|---|
| Diarrhea | Increase 4-6 stools/day over baseline |
| Vomiting | 3-5 episodes in 24 hours |
| Abdominal pain | Moderate pain limiting daily activities |
| Postural | Moderately symptomatic and limiting daily |
| hypotension | activities or BP <80/40 |

No formal sample size calculation was performed for Stage 1. The number of subjects (n=10) was based on feasibility and was considered sufficient to meet the objectives of the study; which was to determine the tolerability of the treatment across the range of tested doses. For Stage 2, assuming the highest proportion of spontaneous resolution of constipation with no treatment to be 0.10, 34 evaluable subjects who have measurements at both baseline and at the end of the fixed dose period provided 80% power to detect the difference between 0.10 (proportion expected if patients are not treated) and a squalamine (ENT-01) treated proportion of 0.29.

No randomization was performed for Stage 1. During the randomization period of Stage 2, subjects were randomly allocated in equal proportion (1:1) to 1 of 2 double-blind treatment groups in a block size of 4: (1) squalamine (ENT-01) at the identified fixed dose level, or (2) placebo at the identified fixed dose level.

Adverse events were coded using the current version of MedDRA. Severity of AEs were assessed by investigators according to CTCAE (v4.03): Grade 1 is labeled as Mild, Grade 2 as Moderate, and Grade 3 and above as Severe. AEs that have a possible, probable or definite relationship to study drug were defined to be related to the study drug while others were defined as “not related”. The number (percentage) of subjects who experienced an AE during escalation and fixed dosing periods were summarized by dose level and overall for each stage. The denominator for calculating the percentages were based on the number of subjects ever exposed to each dose and overall.

Effect on Bowel Function:

Cumulative responder rates of bowel function are shown in FIG. 3A. In Stage 1 (single dose), cumulative response rate increased in a dose-dependent fashion from 25% at 25 mg to a maximum of 80% at 200 mg.

In Stage 2 (daily dosing), the response rate increased in a dose-dependent fashion from 26% at 75 mg to 85.3% at 250 mg. The dose required for a bowel response was patient-specific and varied from 75 mg to 250 mg. Median efficacious dose was 100 mg. Average CSBM/week increased from 1.2 at baseline to 3.8 at fixed dose (p=2.3×10⁻⁸) and SBM increased from 2.6 at baseline to 4.5 at fixed dose (p=6.4×10⁻⁶) (Table 6). Use of rescue medication decreased from 1.8/week at baseline to 0.3 at fixed dose (p=1.33×10⁻⁵). Consistency based on the Bristol stool scale also improved, increasing from mean 2.7 to 4.1 (p=0.0001) and ease of passage increased from 3.2 to 3.7 (p=0.03). Subjective indices of wellbeing (PAC-QOL) and constipation symptoms (PAC-SYM) also improved during treatment (p=0.009 and p=0.03 respectively).

TABLE 6
Stool related indices Stage 2 (Dosed patients, n = 34)

| | Baseline | Fixed dose | |
|---|---|---|---|
| | (mean, SD) | (mean, SD) | P-value |
| CSBM* | 1.2 (0.90) | 3.8 (2.40) | 2.3 × 10⁻⁸ |
| SBM* | 2.6 (1.45) | 4.5 (2.21) | 6.4 × 10⁻⁶ |
| Suppository use* | 1.8 (1.92) | 0.3 (0.67) | 1.33 × 10⁻⁵ |
| Consistency*** | 2.7 (1.20) | 4.1 (2.13) | 0.0001 |
| Ease of passage** | 3.2 (0.73) | 3.7 (1.19) | 0.03 |
| PAC-QOL total | 1.4 (0.49) | 1.2 (0.59) | 0.009 |
| PAC-SYM | 1.3 (0.45) | 1.1 (0.49) | 0.03 |

*weekly average;
**Ease of evacuation scale, where 1-manual disimpaction and 7 = incontinent;
***Bristol stool scale 1-7, where 1 = separate hard lumps and 7 = liquid consistency

The dose that proved efficacious in inducing a bowel response was strongly related to constipation severity at baseline (p=0.00055) (FIG. 3B); patients with baseline constipation of <1 CSBM/week required higher doses for a response (mean 192 mg) than patients with ≥1 CSBM/week (mean 120 mg).

While the improvement in most stool-related indices did not persist beyond the treatment period, CSBM frequency remained significantly above baseline value (Table 7).

TABLE 7
Reversal of stool indices to baseline
during the wash-out period (Stage 2)

| | | | | | P-value |
|---|---|---|---|---|---|
| | Baseline | Fixed dose | | Wash-out | (wash-out |
| | (Mean, SD) | (Mean, SD) | | (Mean, SD) | vs. baseline) |
| CSBM | 1.2 (0.90) | 3.8 | (2.4) | 1.8 (1.19) | 0.01 |
| SBM | 2.6 (1.45) | 4.5 | (2.21) | 3.2 (1.80) | 0.16 |
| Ease | 3.2 (0.73) | 3.7 | (1.19) | 3.3 (0.81) | 0.78 |
| Consistency | 2.7 (1.20) | 4.1 | (2.13) | 2.8 (1.39) | 0.85 |
| Rescue meds | 1.8 (1.92) | 0.3 | (0.67) | 1.0 (1.40) | 0.13 |
| PAQ-QOL | 1.4 (0.49) | 1.2 | (0.59 | 1.2 (0.63) | 0.04 |
| PAQ-SYM | 1.3 (0.45) | 1.1 | (0.49) | 1.1 (0.60) | 0.11 |

The primary efficacy outcome variable was whether or not a subject was a “success” or “failure”. This is an endpoint based on subject diary entries for the “fixed dose” period prior to the endpoint assessment defined as average complete stool frequency increase by 1 or more over baseline, or 3 or more complete spontaneous stools/week. The subject was deemed a “success” if s/he met one or more of the criteria listed above, otherwise the subject was deemed a “failure”. The primary analysis was based on all subjects with a baseline assessment and an assessment at the end of the “fixed-dose” period and was a comparison of the proportion of successes with 0.10 (the null hypothesis corresponding to no treatment effect).

The proportion of subjects for whom the drug was a success was estimated with a binomial point estimate and corresponding 95% confidence interval. A secondary analysis compared the proportions of subjects who are deemed a success at the end of the randomized fixed-dose period between those randomized to the squalamine (ENT-01) arm and those randomized to the placebo arm. A Fisher's exact test was used to compare the proportions of subjects who were deemed a success at the end of randomization period between the two randomized arms.

Subgroup Analysis:

Fifteen patients were randomized to treatment (n=6) or placebo (n=9) after the fixed dose period. During the 4-6 days of randomized treatment, the mean CSBM frequency in the treatment group remained higher than baseline as compared to those receiving placebo who returned to their baseline values (Table 8).

TABLE 8
CSBM frequency in the randomized cohort

| CSBM/week | Baseline | Fixed dose | Randomized | Washout |
|---|---|---|---|---|
| Treatment (n = 6) | 0.8 | 3.2 | 2.4 | 0.9 |
| Placebo (n = 9) | 1.6 | 3.3 | 1.4 | 1.6 |

CSBM increased in both groups during the treatment period and remained high in the treatment group during the randomized period but fell to baseline values in the placebo group.

Pharmakokinetics:

PK data were collected on the 10 patients enrolled in Stage 1 and 10 patients enrolled in Stage 2 to determine the extent of systemic absorption. In Stage 1, PK data were obtained at each visit, pre-medication, at 1, 2, 4, 8 and 24 hours (Table 9). In Stage 2, PK was measured on days 1 and 6 of the randomization period pre-medication, at 1, 2, 4 and 8 hours (Table 10). Based on the pharmacokinetic behavior of intravenously administered squalamine determined in prior clinical studies it is estimated that squalamine (ENT-01) exhibited oral bioavailability of less than 0.3% (Bhargava et al. 2001; Hao et al. 2003).

TABLE 9
Pharmacokinetics of orally administered squalamine (ENT-01) in Stage 1.
Stage 1

| | | | Tₘₐₓ (hour) | T_{1/2} | | |
|---|---|---|---|---|---|---|
| Dose | # of | Cₘₐₓ | (Median | (hours) | AUC_{0-8 hr} | AUC_{0-16 hr} |
| (mg) | patients | (ng/ml) | Value) | (n) | (ng * hour/ml | (ng * hour/ml |
| 25 | 9 | 2.84 | 1.0 | 2.6 (3) | 10.8 | 19.6 |
| 50 | 10 | 3.73 | 2.0 | 3.4 (3) | 18.5 | 33.1 |
| 75 | 9 | 4.33 | 2.0 | 2.8 (2) | 18.4 | 29.8 |
| 100 | 9 | 6.18 | 2.0 | 3.9 (5) | 29.6 | 51.5 |
| 125 | 9 | 9.63 | 2.0 | 3.9 (4) | 43.1 | 77.7 |
| 150 | 7 | 6.27 | 2.0 | 5.6 (4) | 31.5 | 64.0 |
| 175 | 7 | 10.3 | 2.0 | 9.1 (6) | 49.7 | 91.2 |
| 200 | 6 | 15.1 | 2.0 | 9.0 (5) | 78.3 | 157 |

TABLE 10
Pharmacokinetics of orally administered squalamine (ENT-01) in Stage 2.
Stage 2

| | | | Tₘₐₓ | T_{1/2} | |
|---|---|---|---|---|---|
| Dose | # of patients | Cₘₐₓ | (hour) | (hours) | AUC_{0-8 hr} |
| (mg) | (2 visits each) | (ng/ml) | (Median Value) | (n) | (ng*hour/ml |
| 75 | 1 | 10.0 | 3.0 | 5.5 (1) | 59.0 |
| 100 | 4 | 17.7 | 1.0 | 4.8 (5) | 70.3 |
| 125 | | | | | |
| 150 | | | | | |
| 175 | 5 | 11.8 | 2.0 | 10 (6) | 66.8 |

The mean Cₘₐₓ, Tₘₐₓ, T_{1/2} and AUC of the squalamine ion following squalamine (ENT-01) oral dosing for Stage 1 patients was measured. The PK analyses are only approximate, as the lower limit of the validated concentration range was 10 ng/ml; most of the measured concentrations fell below that value. The mean Cₘₐₓ, Tₘₐₓ, T_{1/2} and AUC of the squalamine ion following squalamine (ENT-01) oral dosing for Stage 2 patients was also measured. The PK analyses are only approximate, as the lower limit of the validated concentration range was 0.5 ng/ml.

CNS Symptoms in Stage 2:

An exploratory analysis was done with respect to the sleep data, the body temperature data, mood, fatigue, hallucinations, cognition and other motor and non-motor symptoms of PD. Continuous measurements within a subject were compared with a paired t-test and continuous measurements between subject groups were compared with a two-group t-test. Categorical data were compared with a chi-squared test or a Fisher's exact test if the expected cell counts are too small for a chi-squared test.

CNS Symptoms:

CNS symptoms were evaluated at baseline and at the end of the fixed dose period and the wash-out period (Table 11). Total UPDRS score was 64.4 at baseline, 60.6 at the end of the fixed dose period and 55.7 at the end of the wash-out period (p=0.002); similarly, the motor component of the UPDRS improved from 35.3 at baseline to 33.3 at the end of fixed dose to 30.2 at the end of wash-out (p=0.006). MMSE improved from 28.4 at baseline to 28.7 during treatment and to 29.3 during wash-out (p=0.0006). BDI-II decreased from 10.9 at baseline to 9.9 during treatment and 8.7 at wash-out (p=0.10). PDHQ improved from 1.3 at baseline to 1.8 during treatment and 0.9 during wash-out (p=0.03). Hallucinations were reported by 5 patients at baseline and delusions in 1 patient. Both hallucinations and delusions improved or disappeared in 5 of 6 patients during treatment and did not return for 4 weeks following discontinuation of squalamine (ENT-01) in 1 patient and 2 weeks in another. Unlike stool-related indices, the improvement in many CNS symptoms persisted during wash-out.

TABLE 11
Effect of Squalamine (ENT-01) on neurological symptoms (n = 34)

| | Baseline | | Fixed dose | | | Wash-out | | |
|---|---|---|---|---|---|---|---|---|
| UPDRS | (Mean, SD) | | (Mean, SD) | | P-value | (Mean, SD) | | P-value |
| Part 1 (NMS) | 11.6 | (6.51) | 10.6 | (6.18)) | 0.28 | 9.5 | (5.27) | 0.06 |
| Part 2 (Daily living) | 14.9 | (8.11) | 14.7 | (9.02) | 0.77 | 14.1 | (8.21) | 0.40 |
| Part 3 (Motor) | 35.3 | (14.35) | 33.3 | (15.20) | 0.13 | 30.2 | (13.23) | 0.005 |
| Total | 64.4 | (23.72) | 60.6 | (25.60) | 0.09 | 55.7 | (23.69) | 0.002 |
| MMSE | 28.4 | (1.75) | 28.7 | (1.9) | 0.21 | 29.3 | (1.06) | 0.0006 |
| PDHQ | 1.3 | (2.99) | 1.8 | (3.34) | 0.45 | 0.9 | (2.33) | 0.03 |
| BDI-II | 10.9 | (7.12) | 9.9 | (6.45) | 0.14 | 8.7 | (5.19) | 0.10 |

UPDRS: Unified Parkinson's Disease Severity Score;
NMS: Non-motor symptoms;
BDI: Beck Depression Index-II;
MMSE: Mini-mental State exam.
PDHQ: Parkinson's Disease Hallucination Questionnaire

### Conclusions

This Phase 2 trial involving 50 patients with PD assessed the safety of orally administered ENT-01, and the effect on bowel function and neurologic symptoms of PD. In addition, the study aimed to identify a dose of ENT-01 that normalizes bowel function in each patient. The study achieved the objectives of identifying safety and pharmacodynamic responses of ENT01 in PD. In addition, the study is the first proof of concept demonstration that directly targeting αS pharmacologically can achieve beneficial GI, autonomic and CNS responses.

The effective dose ranged between 75 mg and 250 mg, with 85% of patients responding within this range. This dose correlated positively with constipation severity at baseline consistent with the hypothesis that gastrointestinal dysmotility in PD results from the progressive accumulation of αS in the ENS, and that squalamine (ENT-01) can restore neuronal function by displacing αS and stimulating enteric neurons. These results demonstrate that the ENS in PD is not irreversibly damaged and can be restored to normal function.

The prokinetic effect of the aminosterol squalamine appears to occur through local action of the compound on the ENS, since squalamine, the active zwitterion, is not significantly absorbed into the systemic circulation.

### Example 2—Constipation

This prophetic example describes an exemplary method of (i) treating constipation and/or (ii) treating and/or preventing a disorder in which constipation is a known symptom (e.g., a constipation associated disorder) in a subject.

Patients are selected based on the constipation criteria described in Example 1. Patients are grouped based on having a particular constipation associated disorder or having constipation with no underlying disorder. The groups are then subdivided into a control subgroup and a treatment subgroup. A “fixed dose” of an aminosterol or a salt or derivative thereof for each of the patients in the treatment subgroup is determined using the method described in Example 1 and in the application supra. Treatment and wash-out periods mirror Example 1. Patients are monitored for changes in the severity or occurrence of the symptoms. Patients with an underlying disorder are also monitored for changes in other symptoms associated with the disorder. Patients with no underlying disorder are monitored for the development of a constipation associated disorder.

Patients having more severe constipation, e.g., less than 1 spontaneous bowel movement per week, are started at a dose of 75 mg or more. Patients having less severe constipation, e.g., 1 or more SBM/week, are started at a lower dose of aminosterol, e.g., a starting dose of less than 75 mg, for example a dose of 25 mg/day. Thus, the starting aminosterol dose is dependent upon constipation severity. The full aminosterol dosing range is from about 1 to about 500 mg. Once a prokinetic dose has been identified for a patient, the subject is started at that same dose following drug cessation and reintroduction of drug dosing; e.g., there is no need to ramp up dosing once a prokinetic dose for a patient has been identified.

It will be apparent to those skilled in the art that various modifications and variations can be made in the methods and compositions of the present invention without departing from the spirit or scope of the invention. Thus, it is intended that the present invention cover the modifications and variations of this invention, provided they come within the scope of the appended claims and their equivalents.

### REFERENCES

Aarsland et al., “Neuropsychiatric symptoms in patients with Parkinson's disease and dementia: frequency, profile and associated care giver stress,”J. Neurol. Neurosurg. Psychiatry, 78:36-42 (2007).
Agachan et al., “A constipation scoring system to simplify evaluation and management of constipated patients,” Diseases of the Colon and Rectum, 39: 681-685 (1996).
Albert et al., “The Diagnosis of Mild Cognitive Impairment Due to Alzheimer's Disease: Recommendations from the National Institute on Aging—Alzheimer's Association Workgroups on Diagnostic Guidelines for Alzheimer's Disease,” Alzheimer's & Dementia, 7(3):270-279 (2011).
Ahima et al., “Appetite suppression and weight reduction by a centrally active aminosterol.” Diabetes, 51(7): 2099-104 (2002).
Akhter et al., “Squalamine, a novel cationic steroid, specifically inhibits the brush-border Na+/H+ exchanger isoform NHE3.” Am. J. Physiol., 276(1 Pt 1): C136-44 (1999).
Alexander et al., “Membrane surface charge dictates the structure and function of the epithelial na+/h+ exchanger. EMBO J., 30:679-691 (2011).
Andresen et al. Effect of 5 days linaclotide on transit and bowel function in females with constipation-predominant irritable bowel syndrome. Gastroenterology, 133:761-8 (2007).
Antonio-Rubio et al., “Abnormal thermography in Parkinson's disease,” Parkinsonism Relat. Disord., 21:852-7 (2015).
Auyeung et al., “Ten year survival and outcomes in a prospective cohort of new onset Chinese Parkinson's disease patients,” J. Neurol. Neurosurg. Psychiatry, 83:607-11 (2012).
Berg et al., “MDS Research Criteria for Prodromal Parkinson's Disease,” Mov. Disord., 30:1600-1611 (2015).
Bhargava et al., “A phase I and pharmacokinetic study of squalamine, a novel antiangiogenic agent, in patients with advanced cancers,” Clin. Cancer Res., 7(12): 3912-9 (2001).
Braak et al., “Idiopathic Parkinson's disease: possible routes by which vulnerable neuronal types may be subject to neuroinvasion by an unknown pathogen,” J Neural Transm (Vienna), 110:517-36 (2003a).
Braak et al., “Staging of brain pathology related to sporadic Parkinson's disease,” Neurobiol Aging, 24:197-211 (2003b).
Braak et al., “Gastric alpha-synuclein immunoreactive inclusions in Meissner's and Auerbach's plexuses in cases staged for Parkinson's disease-related brain pathology,” Neuroscience letters, 396:67-72 (2006).
Breen et al., “Sleep and circadian rhythm regulation in early Parkinson disease,” JAMA Neurol., 71:589-95 (2014).
Breen, D. P. & Lang, A. E., “Tracking the Course of Prodromal Parkinson's Disease,” Brain, 140:259-262 (2017).
Chang et al., “A Meta-Analysis of Genome-Wide Association Studies Identifies 17 New Parkinson's Disease Risk Loci,” Nat. Genet., 49:1511-1516 (2017).
B. Coffin & C. Causse, “Constipation assessment scales in adults: a literature review including the new Bowel Function Index,” Exp. Rev. of Gastroenterology & Hepatology, 5(5): 601-6013 (Jan. 10, 2014).
Cordell et al., “Alzheimer's Association Recommendations for Operationalizing the Detection of Cognitive Impairment During the Medicare Annual Wellness Visit in a Primary Care Setting,” Alzheimer's & Dementia, 9(2):141-150 (2013).
Connolly et al., “Squalamine lactate for exudative age-related macular degeneration,” Ophthalmol. Clin. North Am., 19:381-91, (2006).
Darweesh et al., “Trajectories of Prediagnostic Functioning in Parkinson's Disease,” Brain, 140:429-441 (2017).
Delgado et al., “Neuroprotective effect of vasoactive intestinal peptide (VIP) in a mouse model of Parkinson's disease by blocking microglial activation.” Faseb. J., 17(8): 944-6 (2003).
Diederich et al., “Hallucinations in Parkinson disease,” Nat. Rev. Neurol., 5:331-42 (2006).
Philippe Ducrotté & Christian Caussé, “The Bowel Function Index: a new validated scale for assessing opioid-induced constipation,” Current Medical Research and Opinion, 28:3, 457-466 (2012).
Fahn et al., Members of the UPDRS Development Committee. UNIFIED PARKINSON'S DISEASE RATING SCALE. Florham Park, N.J.: Macmillan Health Care Information (1987).
Frank et al., “Psychometric validation of a constipation symptom assessment questionnaire, Scand. J. Gastroenterol., 34:870-7 (1999).
Friedman J H, Akbar U., “Psychosis in Parkinson's disease: unexplained observations in a seemingly simple model,” Expert Rev. of Neurotherapeutics, 16:595-6 (2016).
Genaidy et al., “Effect of squalamine on iris neovascularization in monkeys.” Retina, 22(6): 772-8 (2002).
Genesis, A., “Squalamine trial for the treatment of fibrodysplasia ossificans progressiva initiated”, Angiogenesis Weekly, 8:45 (2002).
Gjerstad et al., “Excessive daytime sleepiness in Parkinson disease: is it the drugs or the disease?” Neurology, 67:853-8 (2006).
Goetz C G, Stebbins G T., “Risk factors for nursing home placement in advanced Parkinson's disease,” Neurology, 43:2227-9 (1993).
Gonzalez-Rey et al., “Therapeutic effect of vasoactive intestinal peptide on experimental autoimmune encephalomyelitis: down-regulation of inflammatory and autoimmune responses,” Am. J. Pathol., 168(4): 1179-88 (2006).
Gressens et al., “Vasoactive intestinal peptide prevents excitotoxic cell death in the murine developing brain,” J. Clin. Invest., 100(2): 390-7 (1997).
Hao et al., “A Phase I and pharmacokinetic study of squalamine, an aminosterol angiogenesis inhibitor,” Clin. Cancer Res., 9(7): 2465-71 (2003).
Heaton et al., Defecation frequency and timing, and stool form in the general population: a prospective study. Gut, 33:818-24 (1992).
Herbst et al., “A phase IIIA trial of continuous five-day infusion of squalamine lactate (MSI-1256F) plus carboplatin and paclitaxel in patients with advanced non-small cell lung cancer,” Clin. Cancer Res., 9(11): 4108-15 (2003).
Higgins et al., “Squalamine improves retinal neovascularization,” Invest. Ophthalmol. Vis. Sci., 41(6): 1507-12 (2000).
Higgins et al., “Regression of retinopathy by squalamine in a mouse model,” Pediatr. Res., 56(1): 144-9 (2004).
Holmqvist et al., “Direct evidence of Parkinson pathology spread from the gastrointestinal tract to the brain in rats,” Acta Neuropathol., 128:805-20 (2014).
Hughes et al., “Accuracy of clinical diagnosis of idiopathic Parkinson's disease: a clinico-pathological study of 100 cases,” J. Neurol. Neurosurg. Psychiatry, 55:181-4 (1992).
Hughes et al., “Associations of Probable REM Sleep Behavior Disorder, Constipation, and Hyposmia with PD” (2017), in Movement Disorder Society: Proceedings of the International Congress of Parkinson's Disease and Movement Disorders; Marsili et al., 2018. Diagnostic Criteria for Parkinson's Disease: From James Parkinson to the Concept of Prodromal Disease. Front. Neurol., Online 23 Mar. 2018.
Jack et al., “Introduction to the Recommendations from the National Institute on Aging—Alzheimer's Association Workgroups on Diagnostic Guidelines for Alzheimer's Disease,” Alzheimer's & Dementia, 7(3):257-262 (2011).
Jennings et al., “Hyposmic and Dopamine Transporter-Deficit Prodromal Cohort,” JAMA Neurol., 74:933-940 (2017).
Jorm, A. F., “The Informant Questionnaire on Cognitive Decline in the Elderly (IQCODE): A Review,” International Psychogeriatrics, 16:1-19 (2004).
Kirkevold, O. & Selbaek, G., “The Agreement Between the MMSE and IQCODE Tests in a Community-Based Sample of Subjects Aged 70 Years or Older Receiving In-Home Nursing: An Explorative Study,” Dement Geriatr. Cogn. Dis. Extra, 5(1):32-41 (2015).
Lewis et al., “Stool form scale as a useful guide to intestinal transit time,” Scand. J. Gastroenterol, 32:920-4 (1997).
Li et al., “Squalamine and cisplatin block angiogenesis and growth of human ovarian cancer cells with or without HER-2 gene overexpression,” Oncogene, 21(18): 2805-14 (2002).
Li et al., “GLP-1 receptor stimulation preserves primary cortical and dopaminergic neurons in cellular and rodent models of stroke and Parkinsonism,” Proc. Natl. Acad. Sci. USA, 106(4): 1285-90 (2009).
Lin et al., “Risk of Parkinson's disease following severe constipation: a nationwide population-based cohort study,” Parkinsonism Relat Disord, 20:1371-5 (2014).
Locke et al., “American Gastroenterological Association Medical Position Statement: guidelines on constipation,” Gastroenterology, 119 (6): 1761-6 (December 2000).
MacDonald, D. (1995). “Squalamine for STDs.” Abstract no F7 35th ICAAC conference.
Madrid-Navarro et al., “Multidimensional Circadian Monitoring by Wearable Biosensors in Parkinson's Disease,” Front. Neurol., 9:157 (2018).
Mahlknecht et al., “Prodromal Parkinson's Disease as Defined per MDS Research Criteria in the General Elderly Community,” Mov. Disord., 31:1405-1408 (2016).
Marquis et al., Development and validation of the Patient Assessment of Constipation Quality of Life questionnaire. Scand J Gastroenterol, 40:540-51 (2005).
Marsili et al., “Diagnostic Criteria for Parkinson's Disease: From James Parkinson to the Concept of Prodromal Disease,” Front. Neurol., Online 23 Mar. 2018.
McKhann et al., “The Diagnosis of Dementia Due to Alzheimer's Disease: Recommendations from the National Institute on Aging—Alzheimer's Association Workgroups on Diagnostic Guidelines for Alzheimer's Disease,” Alzheimer's & Dementia, 7(3):263-269 (2011).
McMillan S C, Williams F A, “Validity and reliability of the Constipation Assessment Scale,” Cancer nursing., 12:183-188 (1989).
Mearin et al., “Bowel Disorders,” Gastroenterology, 150(6):1393-1407 (2016).
Moore et al., “Squalamine: an aminosterol antibiotic from the shark,” Proc. Natl. Acad. Sci. USA, 90(4): 1354-8 (1993).
S. Morairty, “Detecting Neurodegenerative Diseases Before Damage Is Done,” SRI International (Jul. 26, 2013) (https://www.sri.com/blog/detecting-neurodegenerative-diseases).
Ondo et al., “Daytime sleepiness and other sleep disorders in Parkinson's disease,” Neurology, 57:1392-6 (2001).
Ondo et al., “Placebo-controlled trial of lubiprostone for constipation associated with Parkinson disease,” Neurology, 78:1650-4 (2012).
Ortiz-Tudela et al., “Ambulatory circadian monitoring (ACM) based on thermometry, motor activity and body position (TAP): a comparison with polysomnography,” Physiol. Behav., 126:30-8 (2014).
Pagano, G., “Imaging in Parkinson's Disease,” Clin. Med., 16:371-375 (2016).
Palsetia et al., “The Clock Drawing Test versus Mini-mental Status Examination as a Screening Tool for Dementia: A Clinical Comparison,” Indian J Psychol Med, 40:1-10 (2018).
Papapetropoulos et al., “A questionnaire-based (UM-PDHQ) study of hallucinations in Parkinson's disease,” BMC Neurol, 8:21 (2008).
Perni et al., “A natural product inhibits the initiation of alpha-synuclein aggregation and suppresses its toxicity,” PNAS, USA, 114:E1009-E17 (2017).
Phillips et al., “Alpha-synuclein-immunopositive myenteric neurons and vagal preganglionic terminals: autonomic pathway implicated in Parkinson's disease?” Neuroscience, 153:733-50 (2008).
Plassman et al., “Prevalance of Cognitive Impairment Without Dementia in the United States,” Ann. Intern. Med., 148(6):427-434 (2009).
Postuma et al., “The New Definition and Diagnostic Criteria of Parkinson's Disease,” Lancet Neurol., 15:546-548 (2016).
Postuma et al., “The New Definition and Diagnostic Criteria of Parkinson's Disease,” Lancet Neurol., 15:546-548 (2016).
Rocca et al., “The Role of T1-Weighted Derived Measures of Neurodegeneration for Assessing Disability Progression in Multiple Sclerosis,” Front Neurol., 8:433 (Sep. 4, 2017).
Rao et al., “Aminosterols from the dogfish shark Squalus acanthias,” J. Nat. Prod., 63(5): 631-5 (2000).
Salmi et al., “New stereoselective titanium reductive amination synthesis of 3-amino and polyaminosterol derivatives possessing antimicrobial activities,” Eur. J. Med. Chem., 43(3): 540-7 (2008).
Salmi et al., “Squalamine: an appropriate strategy against the emergence of multidrug resistant gram-negative bacteria?” PLoS ONE, 3(7): e2765 (2008).
Sarabia et al., “Circadian rhythm of wrist temperature in normal-living subjects A candidate of new index of the circadian system,” Physiol. Behav., 95:570-80 (2008).
Schiller and Bittner, “Potentiation of platinum antitumor effects in human lung tumor xenografts by the angiogenesis inhibitor squalamine: effects on tumor neovascularization,” Clin. Cancer Res., 5(12): 4287-94 (1999).
Selinsky et al., “Squalamine is not a proton ionophore,” Biochim. Biophys. Acta., 1464(1): 135-41 (2000).
Selinsky et al., “The aminosterol antibiotic squalamine permeabilizes large unilamellar phospholipid vesicles,” Biochim. Biophys. Acta., 1370(2): 218-34 (1998).
Shehata et al., “Neuronal stimulation induces autophagy in hippocampal neurons that is involved in AMPA receptor degradation after chemical long-term depression,” J. Neurosci., 32:10413-22 (2012).
Sills et al., “Squalamine inhibits angiogenesis and solid tumor growth in vivo and perturbs embryonic vasculature,” Cancer Res., 58(13): 2784-92 (1998).
Sokoloff et al., “Adjunctive therapy for men with high risk localized and locally advanced prostate cancer: targeting disseminated tumor cells,” J. Urol., 172(6 Pt 2): 2539-44 (2004).
Steer et al., “Use of the Beck Depression Inventory-II with depressed geriatric inpatients,” Behav Res Ther, 38:311-8 (2000).
Steinberg, B. E. and S. Grinstein, “Pathogen destruction versus intracellular survival: the role of lipids as phagosomal fate determinants,” J. Clin. Invest., 118(6): 2002-11 (2008).
Stiasny-Kolster et al., “The REM sleep behavior disorder screening questionnaire—a new diagnostic instrument,” Movement disorders: Official J. of the Movement Dis. Soc., 22:2386-93 (2007).
Jon Stoessl, “Neuroimaging in the early diagnosis of neurodegenerative disease,” Transl. Neurodegener., 1: 5 (2012).
Steer et al., “Use of the Beck Depression Inventory-II with depressed geriatric inpatients,” Behav. Res. Ther., 38:311-8 (2000).
Sumioka et al., “TARP phosphorylation regulates synaptic AMPA receptors through lipid bilayers,” Neuron, 66(5): 755-67 (2009).
Stolzenberg et al., “A Role for Neuronal Alpha-Synuclein in Gastrointestinal Immunity,” J. Innate Immun., 9:456-63 (2017).
Svensson et al., “Does vagotomy reduce the risk of Parkinson's disease: The authors reply” Ann. Neurol., 78:1012-3 (2015).
Tang et al., “Loss of mTOR-Dependent Macroautophagy Causes Autistic-like Synaptic Pruning Deficits,” Neuron, 83(5):1131-1143 (2014).
Tirassa et al., “CCK-8 prevents the development of kindling and regulates the GABA and NPY expression in the hippocampus of pentylenetetrazole (PTZ)-treated adult rats,” Neuropharmacology, 48(5): 732-42 (2005).
US 2005/0261508A1 for “Aminosterol Compounds useful as inhibitors of the sodium/proton exchanger (NHE), pharmaceutical methods, and compositions employing such inhibitors, and processes for evaluating the NHE-inhibitory efficacy of compounds,” Zasloff et al., Published Nov. 24, 2005.
US 2006/0166950A1 for “Treatment of neovascularization disorders with squalamine,” Zasloff et al., Published Jun. 27, 2006.
US 2006/0183928A1 for “Aminosterol Compounds useful as inhibitors of the sodium/proton exchanger (NHE), pharmaceutical methods, and compositions employing such inhibitors, and processes for evaluating the NHE-inhibitory efficacy of compounds”, Published Aug. 17, 2006.
US 2007/10504A1 for “Polymorphic and Amorphous salt forms of squalamine dilactate” Chellquist, Doubleday, Gilbert, Zhang, McLane, Armbruster, Levitt, Published Jan. 11, 2007.
US 2011/0097303 for “Methods and Compositions for Treating and Preventing Viral Infections,” published Apr. 28, 2011, Zasloff.
U.S. Pat. No. 5,192,756 for “Aminosterol antibiotic,” Zasloff, Moore, Wehrli, Issued Mar. 9, 1993.
U.S. Pat. No. 5,637,691 (1993) for “Steroid derivatives, pharmaceutical compositions containing them, and their use as antibiotics and disinfectants”, Frye, Zasloff, Kinney, Moriarty.
U.S. Pat. No. 5,721,226 (1998) for “Methods for treating angiogenesis using squalamine and squalamine steroid derivatives,” Frye, Zasloff, Kinney, Moriarty, Collins.
U.S. Pat. No. 5,733,899 (1998) for “Methods for treating infections using steroid based pharmaceutical compositions,” Frye, Zasloff, Kinney, Moriarty, Collins.
U.S. Pat. No. 5,763,430 (1998) for “Method of treating a viral infection by administering a steroid compound,” Zasloff.
U.S. Pat. No. 5,792,635 (1998) for “Method of inhibiting the sodium-proton exchanger NHE3 and method of inhibiting growth by administering squalamine,” Zasloff.
U.S. Pat. No. 5,795,885 (1998) for “Method of Inhibiting proliferation of cells by administering an aminosterol compound,” Zasloff, Shinnar, Kinney, Anderson, Williams, McLane.
U.S. Pat. No. 5,834,453 (1998) for “Methods for the manufacture and use of antimicrobial sterol conjugates,” Regen (Leheigh Univ).
U.S. Pat. No. 5,840,740 (1998) for “Amino sterol compounds and a method of treating infection using the aminosterol compounds,” Zasloff, Shinnar, Kinney, Rao.
U.S. Pat. No. 5,840,936 (1998) for “Aminosterol compounds useful as inhibitors of the sodium/proton exchanger (NHE),” Zasloff, Shinnar, Rao, Kinney.
U.S. Pat. No. 5,847,172 (1998) for “Certain Aminosterol compounds and Pharmaceutical compositions including these compounds,” Zasloff, Shinnar, Kinney, Jones.
U.S. Pat. No. 5,856,535 (1999) for “Amino sterol ester compounds,” Zasloff, Kinney, Jones.
U.S. Pat. No. 5,874,597 (1999) for “Certain Aminosterol compounds and pharmaceutical compositions including these compounds,” Jones, Issued Feb. 23, 1999.
U.S. Pat. No. 5,994,336 (1999) for “Method of inhibiting proliferation of cells by administering an aminosterol compound,” Zasloff, Shinnar, Kinney, Rao, Issued Nov. 30, 1999.
U.S. Pat. No. 6,017,906 (2000) for “Polyamine conjugates for treatment of infection,” Mintz, C S et al Intercardia, Inc., Issued Jan. 25, 2000.
U.S. Pat. No. 6,143,738 (2000) for “Therapeutic uses for an aminosterol compound,” Zasloff, Issued Nov. 7, 2000.
U.S. Pat. No. 6,147,060 (2000) for “Treatment of carcinomas using squalamine in combination with other anti-cancer agents,” Zasloff, Williams, Issued Nov. 14, 2000.
U.S. Pat. No. 6,388,108 (2002) for “Aminosterol compounds and uses thereof,” Rao, Feibush, Kinney, Zasloff, Noecker, Issued May 14, 2002.
U.S. Pat. No. 6,596,712 (2003) for “Treatment of carcinomas using squalamine in combination with other anticancer agents or modalities,” Zasloff, Williams, Sokoloff, Issued Jul. 22, 2003.
U.S. Pat. No. 6,962,909 (2005) for “Treatment of neovascularization disorders with squalamine,” Zasloff, Shinnar, Kinney, Jones, Issued Nov. 8, 2005.
Videnovic A, Golombek D., “Circadian Dysregulation in Parkinson's Disease,” Neurobiol. Sleep Circadian Rhythms, 2:53-8 (2017).
Wald et al., “A multinational survey of prevalence and patterns of laxative use among adults with self-defined constipation,” Alimentary Pharmacology & Therapeutics, 28 (7): 917-930 (Oct. 1, 2008).
Walia et al., “Recent advances in chronic constipation,” Curr. Opin. Pediatr., 21 (5): 661-6 (October 2009).
West et al., “Squalamine increases vagal afferent firing frequency in aging mice,” J. of the Canadian Association of Gatroenterology, 1 (2018).
Wimo et al., “The worldwide economic impact of dementia 2010,” Alzheimer's Dement., 9: 1-11 (2013).
Zahodne et al., “Mood and motor trajectories in Parkinson's disease: multivariate latent growth curve modeling,” Neuropsychology, 26:71-80 (2012).
Zinsmeister et al., “Pharmacodynamic and clinical endpoints for functional colonic disorders: statistical considerations,” Dig. Dis. Sci., 58:509-18 (2013).
Learning About Parkinson's Disease, NIH Nat'l Human Genome Research Inst., https://www.genome.gov/10001217/learning-about-parkinsons-disease/ (last updated Mar. 14, 2014); Parkinson Disease, NIH U.S. Nat'l Library of Med.: Genetics Home Reference, https://ghr.nlm.nih.gov/condition/parkinson-disease #genes (last updated Jul. 3, 2018).
van Marken Lichtenbelt et al., “Evaluation of wireless determination of skin temperature using iButtons,” Physiol Behav, 88:489-97 (2006).
Verdin et al., “Characterization of a common high-affinity receptor for reovirus serotypes 1 and 3 on endothelial cells,” J. Virol., 63(3): 1318-25 (1989).
White et al., “Therapeutic potential of vasoactive intestinal peptide and its receptors in neurological disorders,” CNS Neurol. Disord. Drug Targets, 9(5): 661-6 (2010).
Williams et al., “Squalamine treatment of human tumors in nu/nu mice enhances platinum-based chemotherapies,” Clin. Cancer Res., 7(3): 724-33 (2001).
WO 96/08270 (1996) for “Method for inhibiting sexually transmitted diseases using Magainin antimicrobials or Squalamine Compounds,” Jacob, Zasloff, Williams, Bedi.
Yeung et al., “Membrane phosphatidylserine regulates surface charge and protein localization,” Science, 319(5860): 210-3 (2008).
Yin et al., “Antiangiogenic treatment delays chondrocyte maturation and bone formation during limb skeletogenesis,” J. Bone Miner. Res., 17(1): 56-65 (2002).
Yun et al., “Identification of Squalamine in the Plasma Membrane of White Blood Cells in the Sea Lamprey,” Petromyzon marinus,” J. Lipid Res., 48(12): 2579-2586 (2007).
Zasloff, “Antimicrobial peptides of multicellular organisms,” Nature, 415(6870): 389-95 (2002).
Zasloff et al., “A spermine-coupled cholesterol metabolite from the shark with potent appetite suppressant and antidiabetic properties,” Int. J. Obes. Relat. Metab. Disord., 25(5): 689-97 (2001).
Zasloff et al., “Squalamine as a broad-spectrum systemic antiviral agent with therapeutic potential,” Proc. Natl. Acad. Sci. USA, 108(38): 15978-83 (2011).

What is claimed is:

1. A method of treating, preventing, and/or slowing the onset or progression of constipation and/or a constipation-related symptom in a subject in need comprising administering to the subject a therapeutically effective amount of at least one aminosterol, or a salt or derivative thereof, provided that the administering does not comprise oral administration.

2. The method of claim 1, wherein administering comprises administration selected from nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof.

3. The method of claim 1, wherein administering comprises nasal administration.

4. The method of claim 1, wherein the therapeutically effective amount of the at least one aminosterol or a salt or derivative thereof:
(a) comprises about 0.1 to about 20 mg/kg body weight of the subject; and/or
(b) comprises about 0.1 to about 15 mg/kg body weight of the subject; and/or
(c) comprises about 0.1 to about 10 mg/kg body weight of the subject; and/or
(d) comprises about 0.1 to about 5 mg/kg body weight of the subject; and/or
(e) comprises about 0.1 to about 2.5 mg/kg body weight of the subject; and/or
(f) comprises about 0.001 to about 500 mg/day; and/or
(g) comprises about 0.001 to about 250 mg/day; and/or
(h) comprises about 0.001 to about 125 mg/day; and/or
(i) comprises about 0.001 to about 50 mg/day; and/or
(j) comprises about 0.001 to about 25 mg/day; and/or
(k) comprises about 0.001 to about 10 mg/day; and/or
(l) comprises about 0.001 to about 6 mg/day administered intranasal; and/or
(m) comprises about 0.001 to about 4 mg/day administered intranasal; and/or
(n) comprises about 0.001 to about 2 mg/day administered intranasal; and/or
(o) comprises about 0.001 to about 1 mg/day administered intranasal.

5. The method of claim 1, wherein:
(a) the aminosterol or a salt or derivative thereof is taken on an empty stomach, optionally within two hours of the subject waking; and/or
(b) no food is taken or consumed after about 60 to about 90 minutes of taking the aminosterol or a salt or derivative thereof; and/or
(c) the aminosterol or a salt or derivative thereof is a pharmaceutically acceptable grade of at least one aminosterol or a pharmaceutically acceptable salt or derivative thereof; and/or
(d) the subject is human; and/or
(e) the aminosterol is comprised in a composition further comprising one or more of the following: an aqueous carrier; a buffer; a sugar; and/or a polyol compound; and/or
(f) the subject is a member of a patient population or an individual at risk for developing constipation.

6. The method of claim 1, wherein the aminosterol or the salt or derivative thereof is:
(a) isolated from the liver of Squalus acanthias; and/or
(b) squalamine or a pharmaceutically acceptable salt thereof; and/or
(c) a squalamine isomer; and/or
(d) the phosphate salt of squalamine; and/or
(e) aminosterol 1436 or a pharmaceutically acceptable salt thereof; and/or
(f) an isomer of aminosterol 1436; and/or
(g) the phosphate salt of aminosterol 1436; and/or
(h) comprises a sterol nucleus and a polyamine attached at any position on the sterol, such that the molecule exhibits a net charge of at least +1; and/or
(i) comprises a bile acid nucleus and a polyamine, attached at any position on the bile acid, such that the molecule exhibits a net charge of at least +1; and/or
(j) a derivative modified to include one or more of the following:
(i) substitutions of the sulfate by a sulfonate, phosphate, carboxylate, or other anionic moiety chosen to circumvent metabolic removal of the sulfate moiety and oxidation of the cholesterol side chain;
(ii) replacement of a hydroxyl group by a non-metabolizable polar substituent, such as a fluorine atom, to prevent its metabolic oxidation or conjugation; and
(iii) substitution of one or more ring hydrogen atoms to prevent oxidative or reductive metabolism of the steroid ring system; and/or
(k) a derivative of squalamine modified through medicinal chemistry to improve bio-distribution, ease of administration, metabolic stability, or any combination thereof; and/or
(l) a synthetic aminosterol; and/or
(m) is selected from the group consisting of:

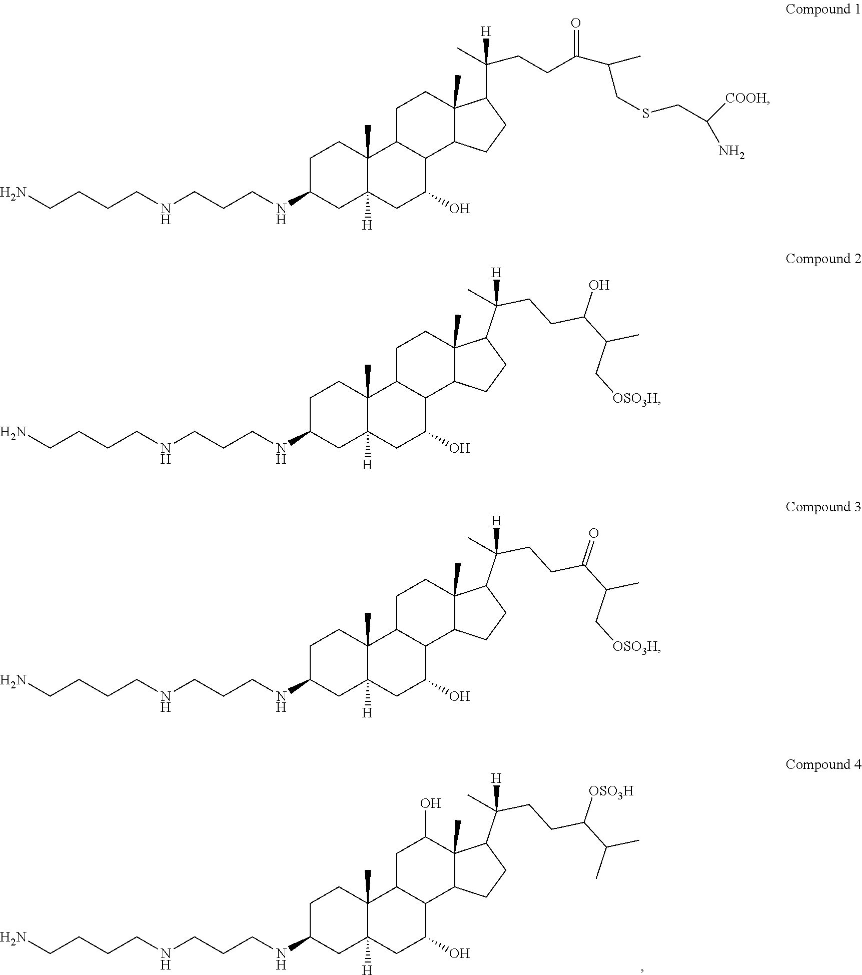

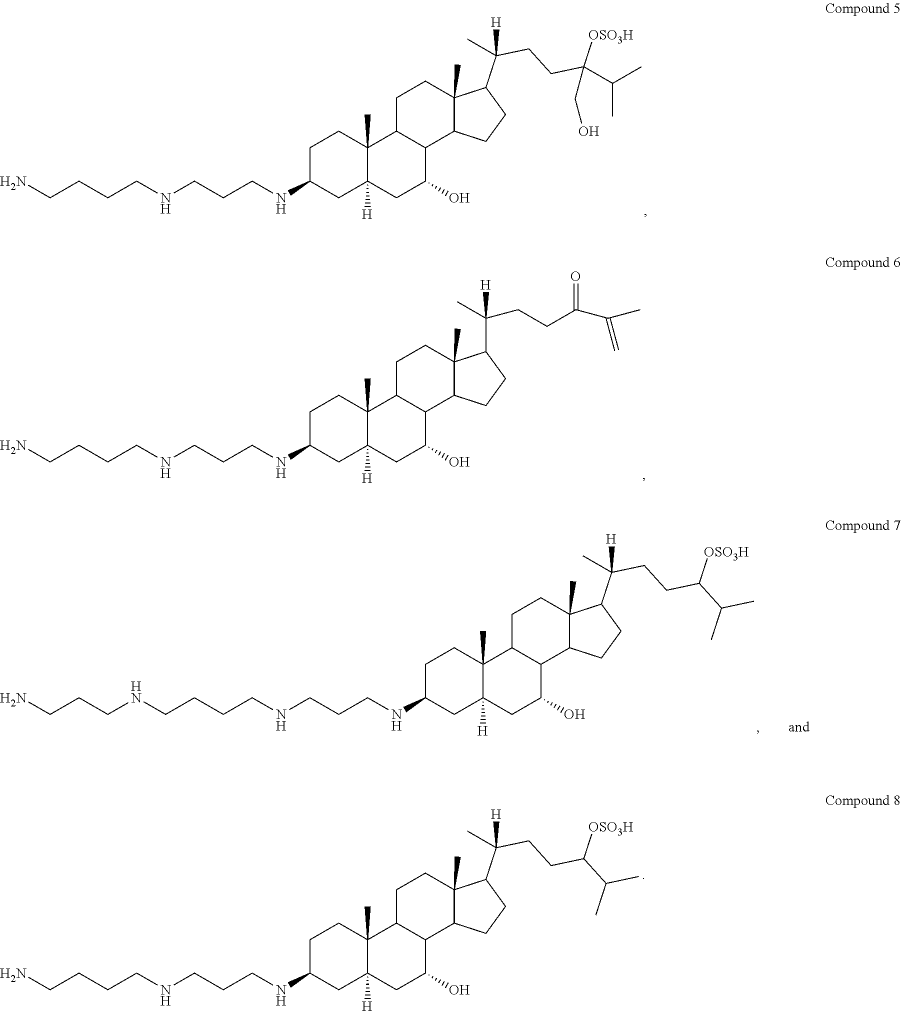

7. A method of treating constipation and/or a constipation-related symptom in a subject in need, comprising:
(a) determining a dose of an aminosterol or a salt or derivative thereof for the subject, wherein the aminosterol dose is determined based on the effectiveness of the aminosterol dose in improving or resolving constipation and/or the constipation-related symptom in the subject;
(b) followed by administering the aminosterol dose to the subject for a period of time, wherein the method comprises:
(i) identifying a constipation-related symptom to be evaluated;
(ii) identifying a starting aminosterol dose for the subject; and
(iii) administering an escalating dose of the aminosterol to the subject over a period of time until an effective aminosterol dose is identified, wherein the effective aminosterol dose is the dose where improvement or resolution of the constipation-related symptom is observed, and fixing the aminosterol dose at that level in that particular subject; and
(c) optionally wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

8. The method of claim 7, wherein the aminosterol or a salt or derivative thereof is administered orally, intranasally, or a combination thereof.

9. The method of claim 8, wherein the aminosterol or a salt or derivative thereof is administered orally and:
(a) the starting aminosterol dose ranges from about 1 mg up to about 175 mg; and/or
(b) the starting oral aminosterol dose is about 25 mg/day; and/or
(c) the dose of the aminosterol or a salt or derivative thereof for the subject following escalation is fixed at a range of from about 1 mg up to about 500 mg; and/or
(d) the dose of the aminosterol or a salt or derivative thereof for the subject following escalation is fixed at a dose of about 1, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, or about 500 mg/day; and/or
(e) the subject experiences moderate constipation or a related symptom, which is defined as a baseline rate of CSBM or SBM in the subject of one or more CSBM or SBM per week, and wherein the starting oral aminosterol dose is from about 10 to about 75 mg/day; and/or
(f) the subject experiences moderate constipation or a related symptom, which is defined as a baseline rate of CSBM or SBM in the subject of one or more CSBM or SBM per week, and wherein the starting oral aminosterol dose is about 10, about 15, about 20, about 25, about 30, about 35, about 40, about 45, about 60, about 65, about 70, or about 75 mg/day; and/or
(g) the subject experiences severe constipation or a related symptom, which is defined as a baseline rate of CSBM or SBM in the subject of less than one CSBM or SBM per week, and wherein the starting oral aminosterol dose is at least about 75 mg/day; and/or
(h) the subject experiences severe constipation or a related symptom, which is defined as a baseline rate of CSBM or SBM in the subject of less than one CSBM or SBM per week, and wherein the starting oral aminosterol dose is from about 75 to about 175 mg/day; and/or
(i) the subject experiences severe constipation or a related symptom, which is defined as a baseline rate of CSBM or SBM in the subject of less than one CSBM or SBM per week, and wherein the starting oral aminosterol dose is about 75, about 80, about 85, about 90, about 95, about 100, about 105, about 110, about 115, about 120, about 125, about 130, about 135, about 140, about 145, about 150 about 155, about 160, about 165, about 170, or about 175 mg/day; and/or
(j) the subject experiences severe constipation or a related symptom, which is defined as a baseline rate of CSBM or SBM in the subject of less than one CSBM or SBM per week, and wherein the starting oral aminosterol dose is at least about 175 mg/day; and/or
(k) the dose of the aminosterol or a salt or derivative thereof is escalated in increments of about 5, about 10, about 15, about 20, about 25, about 30, about 35, about 40, about 45, or about 50 mg; and/or
(l) the dose of the aminosterol or a salt or derivative thereof is escalated in about 25 mg increments; and/or
(m) the aminosterol or a salt or derivative thereof is formulated for oral administration in a composition which is a liquid, capsule, or tablet designed to disintegrate in either the stomach, upper small intestine, or more distal portions of the intestine.

10. The method of claim 8, wherein the aminosterol or a salt or derivative thereof is administered intranasally and:
(a) the starting dose of the aminosterol or a salt or derivative thereof ranges from about 0.001 mg to about 3 mg; and/or
(b) the starting dose of the aminosterol or a salt or derivative thereof is about 0.001, about 0.005, about 0.01, about 0.02, about 0.03, about 0.05, about 0.06, about 0.07, about 0.08, about 0.09, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 1.0, about 1.1, about 1.25, about 1.3, about 1.4, about 1.5, about 1.6, about 1.7, about 1.75, about 1.8, about 1.9, about 2.0, about 2.1, about 2.25, about 2.3, about 2.4, about 2.5, about 2.6, about 2.7, about 2.75, about 2.8, about 2.9, or about 3 mg; and/or
(c) the dose of the aminosterol or a salt or derivative thereof for the subject following escalation is fixed at a range of from about 0.001 mg up to about 6 mg; and/or
(d) the dose of the aminosterol or a salt or derivative thereof for the subject following escalation is fixed at about 0.001, about 0.005, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, or about 6 mg; and/or
(e) the dose of the aminosterol or a salt or derivative thereof for the subject following escalation is a dose which is sub-therapeutic when given orally or by injection; and/or
(f) the dose of the aminosterol or a salt or derivative thereof is escalated in increments of about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg; and/or
(g) the aminosterol or a salt or derivative thereof is formulated for intranasal administration in a composition which is a dry powder or nasal spray or liquid nasal spray.

11. The method of claim 7, wherein:
(a) the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%; and/or
(b) the improvement is measured using a clinically recognized scale or tool; and/or
(c) the constipation-related symptom is frequency of bowel movements, and the improvement or resolution comprises a desired rate of complete spontaneous bowel movement (CSBM) or spontaneous bowel movement (SBM); and/or
(d) the constipation-related symptom is frequency of bowel movements, and the improvement or resolution comprises a rate of CSBM or SBM in the subject of one or more CSBM or SBM per week, 2 or more CSBM or SMB per week, or 3 or more CSBM or SBM per week; and/or
(d) the improvement or resolution comprises an increase in bowel activity, an induction of nausea, an induction of secretory diarrhea, or any combination thereof.

12. The method of claim 7, wherein:
(a) the starting aminosterol dose is based on a baseline rate of complete spontaneous bowel movement (CSBM) or spontaneous bowel movement (SBM) in the subject;
(b) the starting dose of the aminosterol or a salt or derivative thereof is higher if the constipation is severe, where “severe” is defined as less than one CSBM or SBM per week.

13. The method of claim 7, wherein:
(a) the dose of the aminosterol or a salt or derivative thereof is escalated every about 1 to about 14 days; and/or
(b) the dose of the aminosterol or a salt or derivative thereof is escalated every about 3 to 5 days; and/or
(c) the dose of the aminosterol or a salt or derivative thereof is escalated every about 1, about 2, about 3, about 4, about 5, about 6, about 7, about 8, about 9, about 10, about 11, about 12, about 13, or about 14 days; and/or
(d) the dose of the aminosterol or a salt or derivative thereof is escalated about 1×/week, about 2×/week, about every other week, or about 1×/month; and/or
(e) the fixed dose of the aminosterol or a salt or derivative thereof is given once per day, every other day, once per week, twice per week, three times per week, four times per week, five times per week, six times per week, every other week, or every few days; and/or
(f) the fixed dose of the aminosterol or a salt or derivative thereof is given for a few weeks, followed by skipping a few weeks, followed by restarting aminosterol treatment; and/or
(g) the fixed aminosterol dose is incrementally reduced after the fixed dose of aminosterol or a salt or derivative thereof has been administered to the subject for a period of time; and/or
(h) the fixed aminosterol dose is varied plus or minus a defined amount to enable a modest reduction or increase in the fixed dose; and/or
(i) the fixed aminosterol dose is increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

14. The method of claim 7, wherein:
(a) the subject is suffering from a disorder of gastrointestinal motility;
(b) the subject is suffering from a condition or disorder selected from the group consisting of chronic idiopathic constipation, Irritable bowel syndrome, Opioid-induced constipation, and Inflammatory Bowel Disease; and/or
(c) the subject is suffering from a neurodegenerative disease; and/or
(d) the subject is suffering from a neurodegenerative disease wherein the neurodegenerative disease is selected from the group consisting of Parkinson's Disease, Alzheimer's disease (AD), Huntington's chorea and/or Huntington's disease, Multiple Sclerosis, Amyotorphic Lateral Sclerosis (ALS), multiple system atrophy (MSA), schizophrenia, Friedreich's ataxia, vascular dementia, Lewy Body dementia or disease, spinal muscular atrophy, supranuclear palsy, fronto temperal dementia, progressive nuclear palsy, Guadeloupian Parkinsonism, spinocerebellar ataxia, autism, dementia of aging, neuropathy of diabetes, peripheral sensory neuropathy, cerebral palsy, epilepsy, diabetic neuropathy, traumatic head and/or spine injury, stroke, and depression.

15. The method of claim 7, wherein the constipation-related symptom is selected from the group consisting of:
(1) frequency of constipation;
(2) duration of constipation symptoms;
(3) frequency of bowel movements;
(4) fecal incontinence/encopresis;
(5) abdominal pain;
(6) abdominal distension or bloating;
(7) abdominal discomfort;
(8) stomach cramps;
(9) stool consistency;
(10) painful defecation/rectal pain with bowel movement;
(11) rectal burning during or after bowel movement;
(12) rectal bleeding or tearing during or after a bowel movement;
(13) ease of defecation/passing stool;
(14) straining during defecation and/or straining or squeezing to try to pass bowel movements;
(15) incomplete evacuation or bowel movement;
(16) unsuccessful attempts at evacuation;
(17) sensation of incomplete bowel evacuation;
(18) sensation of anorectal obstruction/blockage;
(19) bowel movements that were too hard;
(20) bowel movements that were too small,
(21) change in amount of gas passed rectally;
(22) less frequent bowel movements;
(23) oozing liquid stool;
(24) rectal fullness or pressure;
(25) small stool size;
(26) urge but inability to pass stool; and
(27) personal judgement of constipation.

16. The method of claim 15, wherein the constipation symptom to be evaluated is frequency of bowel movements, and wherein:
(a) the fixed escalated aminosterol dose causes the subject to have a bowel movement; and/or
(b) the method results in an increase in the frequency of bowel movement in the subject over a defined period of time; and/or
(c) the method results in an increase in the frequency of bowel movement in the subject and the increase in the frequency of bowel movement is defined as:
(i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(d) as a result of the method the subject has the frequency of bowel movement recommended by a medical authority for the age group of the subject; and/or
(e) the starting aminosterol dose is determined by the severity of the constipation, wherein:
(i) if the average complete spontaneous bowel movement (CSBM) or spontaneous bowel movement (SBM) is one or less per week, then the starting aminosterol dose is at least about 150 mg; and
(ii) if the average CSBM or SBM is greater than one per week, then the starting aminosterol dose is about 75 mg or less; and/or
(f) wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

17. The method of claim 7, wherein the compound is an aminosterol that can inhibit the formation of actin stress fibers in endothelial cells stimulated by a ligand known to induce stress fiber formation, having the chemical structure of Formula I: 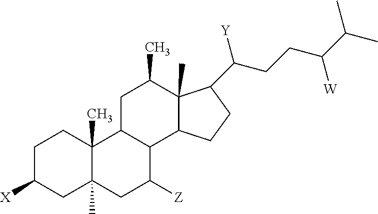
wherein:
W is 24S-OSO₃ or 24R-OSO₃;
X is 3β-H₂N—(CH₂)₄—NH—(CH₂)₃—NH— or 3α-H₂N—(CH₂)₄—NH—(CH₂)₃—NH—;
Y is 20R-CH₃; and
Z is 7α or 7β —OH

18. The method of claim 7, wherein the aminosterol or the salt or derivative thereof is:
(a) isolated from the liver of Squalus acanthias; and/or
(b) squalamine or a pharmaceutically acceptable salt thereof; and/or
(c) a squalamine isomer; and/or
(d) the phosphate salt of squalamine; and/or
(e) aminosterol 1436 or a pharmaceutically acceptable salt thereof; and/or
(f) an isomer of aminosterol 1436; and/or
(g) the phosphate salt of aminosterol 1436; and/or
(h) comprises a sterol nucleus and a polyamine attached at any position on the sterol, such that the molecule exhibits a net charge of at least +1; and/or
(i) comprises a bile acid nucleus and a polyamine, attached at any position on the bile acid, such that the molecule exhibits a net charge of at least +1; and/or
(j) a derivative modified to include one or more of the following:
(i) substitutions of the sulfate by a sulfonate, phosphate, carboxylate, or other anionic moiety chosen to circumvent metabolic removal of the sulfate moiety and oxidation of the cholesterol side chain;
(ii) replacement of a hydroxyl group by a non-metabolizable polar substituent, such as a fluorine atom, to prevent its metabolic oxidation or conjugation; and
(iii) substitution of one or more ring hydrogen atoms to prevent oxidative or reductive metabolism of the steroid ring system; and/or
(k) a derivative of squalamine modified through medicinal chemistry to improve bio-distribution, ease of administration, metabolic stability, or any combination thereof; and/or
(l) a synthetic aminosterol; and/or
(m) is selected from the group consisting of:

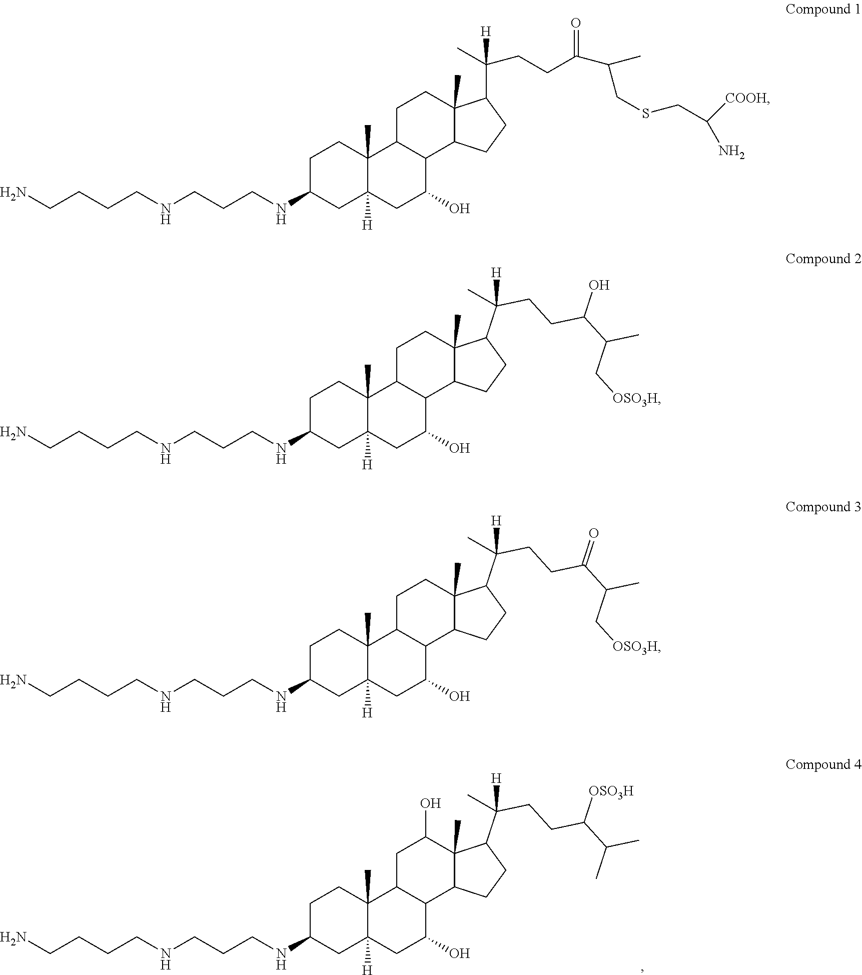

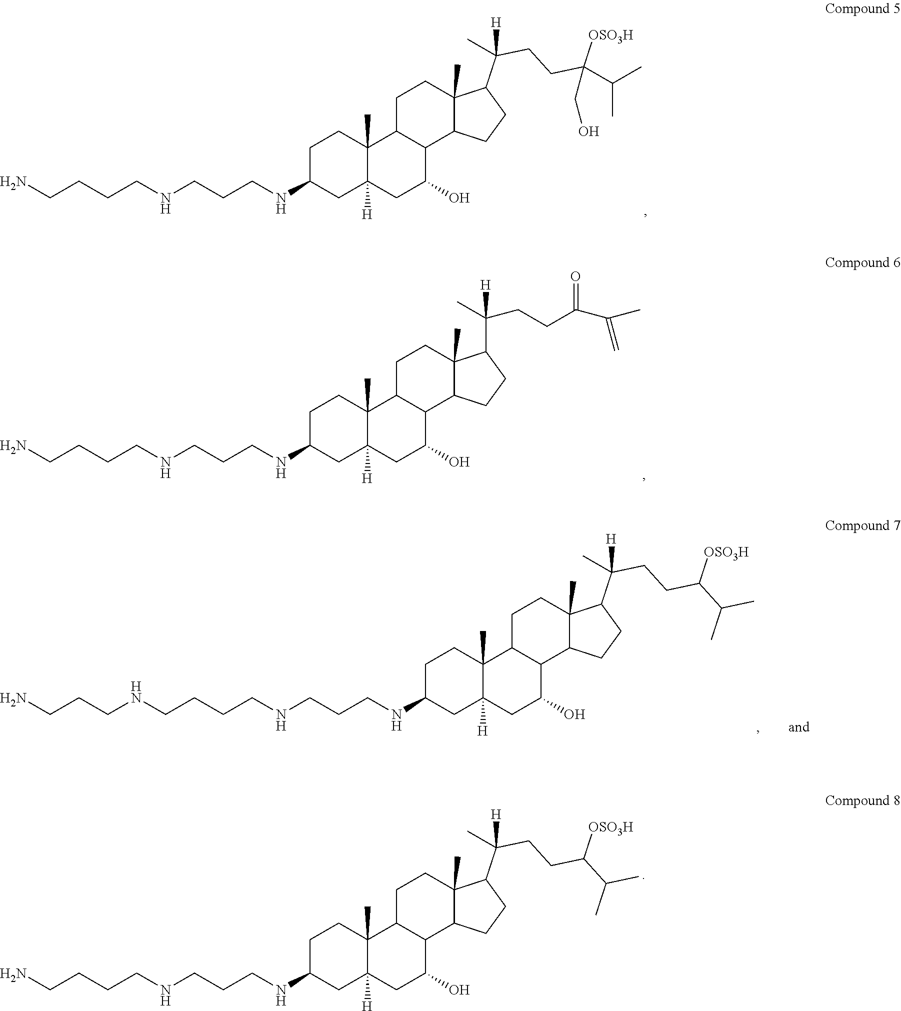

19. The method of claim 7, wherein:
(a) the aminosterol is administered in combination with at least one additional active agent to achieve either an additive or synergistic effect; and/or
(b) the additional active agent is administered via a method selected from the group consisting of concomitantly, as an admixture, separately and simultaneously or concurrently, and separately and sequentially; and/or
(c) the additional active agent is a different aminosterol from that administered in the method of claim 7;
(d) the method of claim 7 comprises a first aminosterol which is aminosterol 1436 or a salt or derivative thereof administered intranasally and a second aminosterol which is squalamine or a salt or derivative thereof administered orally; and/or
(e) the additional active agent is an active agent used to treat constipation or a symptom thereof; and/or
(f) the aminosterol or a salt or derivative thereof is taken on an empty stomach, optionally within two hours of the subject waking; and/or
(g) no food is taken after about 60 to about 90 minutes of taking the aminosterol or a salt or derivative thereof; and/or
(h) the aminosterol or a salt or derivative thereof is a pharmaceutically acceptable grade of at least one aminosterol or a pharmaceutically acceptable salt or derivative thereof; and/or
(i) the aminosterol or a salt or derivative thereof is comprised in a composition further comprising one or more of the following: an aqueous carrier; a buffer; a sugar; and/or a polyol compound.

20. The method of claim 7, wherein:
(a) the subject is a human; and/or
(b) the subject is a member of a patient population at risk for developing constipation or an individual at risk for developing constipation.
